(19) United States
(12) Patent Application Publication
(10) Pub. No.: US 2021/0154161 A1
(43) Pub. Date: May 27, 2021

(54) MODIFIED-RELEASE BUCILLAMINE COMPOSITIONS, KITS, AND METHODS FOR TREATING CYSTINURIA, ARTHRITIS, GOUT, AND RELATED DISORDERS

(71) Applicant: ALTIBIO, INC., Half Moon Bay, CA (US)

(72) Inventors: Patrick Reichenberger, Half Moon Bay, CA (US); Artem Zykovich, Novato, CA (US); Wolfgang Richter, Munich (DE)

(21) Appl. No.: 16/632,755

(22) PCT Filed: Jul. 25, 2018
(86) PCT No.: PCT/US2018/043698

§ 371 Date: Jan. 21, 2020

Related U.S. Application Data
(60) Provisional application No. 62/536,771, filed on Jul. 25, 2017.

(51) Int. Cl. A61K 31/198; A61K 9/48; A61K 9/00; A61K 9/70

(52) U.S. Cl. CPC A61K 31/198; A61K 9/4866; A61K 9/7023; A61K 9/0065; A61K 9/0014; A61K 9/4858

(57) ABSTRACT

The present invention provides methods, compositions, devices, and kits for treating cystinuria and related disorders, in which bucillamine is administered to a patient according to a modified-release strategy that releases bucillamine, preferably in the stomach, in critically-spaced, repeated pulses over time, to provide lower peak plasma levels of total bucillamine, but consistently higher free drug levels in urine, for binding cystine. In particular, use of the present modified-release formulations and systems reduce side effects of bucillamine and achieve efficacy with less frequent administrations and/or lower dosage than immediate-release forms. The present strategies also find use in treating inflammatory conditions, like rheumatoid arthritis, gout, where bucillamine is administered in a different modified-release strategy that releases bucillamine in more closely critically-spaced pulses, again preferably in the stomach, to provide lower peak blood levels of total bucillamine, but higher and steadier blood levels of unbound drug, which improve availability in treating such conditions.

### CROSS-REFERENCE TO RELATED APPLICATIONS

This application claims the benefit of U.S. Provisional Application No. 62/536,771, filed Jul. 25, 2017, incorporated in its entirety by reference.

### RELATED FIELDS

The compositions, kits, and methods described herein generally relate to the fields of medical treatment involving bucillamine. More specifically, methods, systems, and kits are provided for administration of compositions comprising bucillamine to cystinuria patients for modified release in the stomach with reduced peak blood levels, but increased urine levels, thereby extending duration, reducing side effects, increasing efficacy, and improving patient quality of life. Modified release bucillamine compositions, systems, kits, and methods also find use in treating gout and arthritis, where the release strategy is adapted to give higher, steadier plasma levels of the unbound drug.

### BACKGROUND

Cystinuria is an inherited autosomal recessive disease that is characterized by high concentration of the amino acid cysteine in the urine, leading to formation of cystine stones in the kidneys, ureter, and bladder. As the kidneys filter blood to create urine, cystine normally is transported back into the blood. In cystinuria patients, the proximal renal tubules fail to reabsorb filtered cystine, as well as other dibasic amino acids, such as lysine, arginine, and ornithine, leading to accumulation and subsequent precipitation of debilitating cystine stones in the urinary tract. Some patients even accumulate several stones each month. The disorder has a profoundly negative impact on the patient's quality of life. It is estimated that in the United States, about 30,000 people suffer from cystinuria; worldwide, about 1 in 7,000 people may have the disorder. It also is a common disorder in certain dog breeds, in particular, Bulldogs, Newfoundlands, and Labrador Retrievers, and affects a considerable percentage of dogs in the U.S.

While approaches to reduce kidney concentrations of cystine provide a number of treatment options, they may be ineffective in about 85% of cystinuria cases and can have severe side effects. Examples of medications used include immediate-release tiopronin and D-penicillamine (sold under the tradename CUPRIMINE®). Common side effects of D-penicillamine include rashes, arthralgia, leukopenia, gastrointestinal intolerance, nephritic syndrome, and vitamin B6 deficiency, which can be so severe that as many as 50-70% of patients cannot tolerate the drug. In 1988, the FDA approved the use of tiopronin as an alternative that provides lower toxicity; nonetheless, current tiopronin medications are discontinued by about 30% of patients, due to adverse reactions, the need for frequent dosing, and poor efficacy. In addition, the short duration of effect, with current therapies, means frequent dosing and leaves nighttime cystine levels uncontrolled. Ongoing formation and repeated passage of stones may cause serious damage to the kidneys, requiring surgery to remove the stones and, in severe cases, renal transplant. Bucillamine is a di-thiol compound, available in Japan and South Korea for rheumatoid arthritis, and has more recently been studied for gout. In the U.S., bucillamine currently is undergoing clinical trials for cystinuria patients not responding to tiopronin. The trials plan to administer bucillamine tablets three times a day and thus appear not to address the problem of frequent dosing that leads to reduced patient compliance with tiopronin.

Accordingly, there is a need in the art for more effective compositions and methods for administering bucillamine, as well as more effective methods of treating and/or preventing cystinuria and related disorders, particularly with fewer side effects, as well as other conditions treated with bucillamine. The present invention meets these and other needs.

### SUMMARY

The present invention provides methods, compositions, systems, and kits for administering bucillamine a modified release strategy that produces lower peak, but more consistent levels of free bucillamine in the blood, along with higher and more consistent levels of the drug in urine, compared to other strategies. This allows bucillamine to more effectively reduce cystine concentrations in the urine, and thus stone formation in the renal tract, a hallmark of cystinuria. This strategy also finds use in other conditions treated with bucillamine, including inflammatory conditions like gout and rheumatoid arthritis. Use of the present modified-release formulations, systems, kits, and methods, reduces side effects of bucillamine and enhances efficacy, allowing for less frequent dosing and/or lower total daily dosage, thereby improving patient compliance and overall quality of life.

The modified-release strategy of the present invention comprises delivery of multiple pulses (bolus doses) of bucillamine over time, e.g., following a given administration of a modified-release formulation of the invention, where the pulses are released at critically-spaced intervals that provide higher and steadier levels of unbound drug in plasma and urine, and lower and more consistently lower cystine concentrations in urine, compared to approaches with longer or shorter intervals between doses. An effective amount of bucillamine is administered or released in fractions, preferably equal or approximately equal fractions, at intervals of about four to about eight hours, preferably about five to about seven hours, or more preferably about six hours apart, to provide greater efficacy and fewer side effects. This modified-release strategy finds use in cystinuria and other disorders sharing pharmacokinetic characteristics of cystinuria (“cystinuria-related disorders”), e.g., other kidney, bladder, and/or ureter stone disorders, where high urine excretion and/or low blood levels of a therapeutic agent result in improved efficacy and/or reduced toxicity. For example, the present approaches may be useful for disorders where is it desirable to remove a metabolite or other substance, such as a different amino acid, present in the blood through urinary excretion to reduce or correct excess accumulation of the metabolite or substance from the blood or tissue. For treating gout, arthritis, and related conditions, an effective amount of bucillamine is administered or released in fractions at shorter intervals of about two to about six hours, preferably about three to about five hours, or more preferably about four hours apart, again to provide greater efficacy and fewer side effects.

One aspect of the invention thus provides a composition comprising a therapeutic agent and a pharmaceutically acceptable modified-release component for releasing the therapeutic in repeated pulses over time, wherein a first fraction is released at a first time point and a second fraction is released at a second time point about four to about eight hours, or about two to about six hours, after the first time point. Preferably, the modified-release component allows for more than two repeat boluses, wherein a subsequent (n^{th}) fraction is released at a subsequent (n^{th}) time point about four to about eight hours, or about two to about six hours, after a preceding (n−1^{th}) time point. In particular embodiments, the therapeutic agent comprises bucillamine. The bucillamine may be a racemic or non-racemic mixture of bucillamine enantiomers; or substantially one or other of the (+) or (−) enantiomer of bucillamine. In some embodiments, the bucillamine is released in repeated pulses for about 12 to about 24 hours. In some embodiments, the bucillamine is released in repeated pulses for about two days to about a week. In some preferred embodiments, the composition comprises a daily dose of bucillamine, fractions of which are delivered in repeated boluses, more preferably about every six hours over a period of about 24 hours. In some preferred embodiments, the composition comprises a daily dose of bucillamine, fractions of which are delivered in repeated boluses, more preferably about every four hours over a period of about 24 hours. In some preferred embodiments, the composition comprises a weekly dose of bucillamine, fractions of which are delivered in repeated boluses, more preferably about every four hours over a period of about a week. In some embodiments, the modified-release components are arranged in a device or system that is applied to the subject to deliver the therapeutic.

In some embodiments, the composition is formulated for oral administration. The oral formulations generally comprise a pharmaceutically acceptable modified-release component to achieve pulsed release upon passage through the gastrointestinal tract. In preferred embodiments, the modified release comprises a gastro-retentive feature, to prolong stomach residence time of the formulation. In some embodiments, the modified-release component comprises layers of coatings, where the different coatings dissolve at step-wise, critically-spaced times in accordance with aspects of the invention, to release repeated pulses of bucillamine over time, in the same or different areas of the gastrointestinal tract, preferably in the stomach. For example, the layers of coatings may include a coating that dissolves or disintegrates when exposed to conditions in the stomach (a “stomach-dissolving coating”), or one or more coatings that dissolve along different parts of the tract past the stomach, e.g., using an enteric coating. The different coatings may be arranged so as to provide immediate and subsequent repeated release of the therapeutic.

In some embodiments, the modified-release component comprises a matrix comprising differently-dissolving segments, where the different segments dissolve at step-wise, critically-spaced times, to release repeated pulses of bucillamine, in the same or different areas of the gastrointestinal tract. In some embodiments, the modified-release component comprises differently-coated beads, where the different bead coatings dissolve at step-wise, critically-spaced times, to release repeated pulses of bucillamine, in the same or different areas of the gastrointestinal tract. In some embodiments, the modified-release component comprises a capsule of differently-dissolving bands, where the different bands dissolve in at step-wise, critically-spaced times, to release repeated pulses of bucillamine, in the same or different areas of the gastrointestinal tract. In some embodiments, the modified-release component comprises a capsule of differently-releasing plugs, where the different plugs dissolve and release contents of a plugged compartment at step-wise, critically-spaced times, to release repeated pulses of bucillamine, in the same or different areas of the gastrointestinal tract. In some embodiments, the modified-release component comprises a capsule of differently-expanding osmotic-push compartments, where the different compartments first expand due to osmolality differences at step-wise, critically-spaced times, to release repeated pulses of bucillamine, in the same or different areas of the gastrointestinal tract. In some embodiments, the modified-release component comprises a matrix comprising differently-releasing polymers, where the different polymers release bucillamine at step-wise, critically-spaced times, releasing repeated pulses of bucillamine, in the same or different areas of the gastrointestinal tract. In some embodiments, the modified-release component comprises a matrix comprising differently-adhering polymers, where the different polymers adhere to, and release bucillamine in, at step-wise, critically-spaced times, releasing repeated pulses of bucillamine, in the same or different areas of the gastrointestinal tract. In some embodiments, the modified-release component comprises a timed microprocessor, timed to release repeated pulses of bucillamine at step-wise, critically-spaced times, releasing repeated pulses of bucillamine, in the same or different areas of the gastrointestinal tract.

In preferred embodiments, the first and second releases occur in the stomach. In more preferred embodiments, first, second, and third releases occur in the stomach. In even more preferred embodiments, first, second, third, and fourth releases occur in the stomach. Compositions for effecting repeated release in the stomach generally comprise a gastro-retentive feature. In some embodiments, the gastro-retentive feature is at least one selected from a stomach-adhering coating, a floatation system, a sedimentation system, and an expandable system. In some embodiments the composition further comprises an immediate-release component. In some embodiments, the stomach-adhering coating comprises stomach-adhering polymers that partially or fully coat the composition; in some embodiments, the floatation system comprises an effervescent or non-effervescent agent that increases buoyancy of the composition; in some embodiments, the sedimentation system comprises a high density agent in a pellet; in some embodiments, the expandable system comprises a shape-modifying or swellable structure that impedes exit of the composition from the stomach.

In some embodiments, the composition is formulated for topical administration, such as in transdermal or transmucosal formulations. The transdermal formulations generally comprise a pharmaceutically acceptable modified-release component to achieve pulsed release through the skin, and may further comprise one or more skin penetration enhancers. In some embodiments, the transdermal formulation is provided in a skin patch, preferably a skin patch with differently-dissolving microneedles, where the different microneedles dissolve at step-wise, critically-spaced times, to release repeated pulses of bucillamine in accordance with aspects of the invention.

Another aspect of the present invention relates to methods of preparing modified-release formulations and pharmaceutical compositions described herein, as well as preparing dosage forms and delivery systems comprising the compositions. In particular embodiments, the oral dosage form is a tablet or a capsule, comprising a therapeutic agent, such as bucillamine, and a pharmaceutically acceptable modified-release component, preferably including a gastro-retentive feature, as well as one or more excipients suitable for use in oral formulations, such as suitable binders, diluents, disintegrants, lubricants, and stabilizers. In more preferred embodiments, the dosage form is a tablet or capsule for oral administration that provides a therapeutically effective amount of bucillamine for use in methods of the present invention. In a particular embodiment, the bucillamine is formulated into differently-coated beads, e.g., differently-coated microbeads that are filled into a capsule.

Still another aspect of the invention provides methods of using a pharmaceutical composition or delivery system disclosed herein, in oral or other formulations, to deliver a therapeutically effective amount of bucillamine for achieving an extended duration therapeutic effect in a subject in need thereof, such as a patient with cystinuria or a cystinuria-related disorder. In preferred embodiments, the pharmaceutical composition is delivered so as to provide a total daily dose of 1,200 mg or less, or about 14 mg/kg/day or less; or an average total daily dose of about 1,200 mg or less, or about 14 mg/kg/day or less, where the dosage indicates the amount of active ingredient (bucillamine), and the bucillamine is released in repeated, critically-spaced pulses, according to the invention, preferably in the stomach. In particular embodiments, the composition comprises bucillamine in a first treatment dose that achieves an extended duration therapeutic effect, such as reduced urinary cystine concentration effectively below about 250 mg/L, lasting at least about 8 hours, preferably before a second or subsequent treatment dose is administered, thereby extending the interval between doses for the patient.

In preferred embodiments, use of the modified-release formulation or system achieves therapeutic effect for a duration of at least about 10 hours, at least about 14 hours, or at least about 18 hours; and/or the treatment dose is administered no more than twice a day. In more preferred embodiments, the total daily dose used is less than about 1,200 mg/day, such as treatment doses of about 500 mg or less administered twice a day; or, even more preferably, treatment doses of about 300 mg or less administered twice a day. In particular embodiments, the total daily dose may range from about 400 mg to about 800 mg, with individual doses ranging from about 200 mg to about 400 mg for twice daily dosing, or from about 400 mg to about 800 mg for once daily dosing. In other embodiments, the total daily dose may range from about 800 mg to about 1,200 mg, with individual doses ranging from about 400 mg to about 600 mg for twice daily dosing, or from about 800 mg to about 1,200 mg for once daily dosing. In some preferred embodiments, the total daily dose used is less than about 10 mg/kg/day, such as treatment doses of about 5 mg/kg or less administered twice a day; or, even more preferably, treatment doses of about 3 mg/kg or less administered twice a day. In particular embodiments, the total daily dose may range from about 10 mg/kg/day to about 14 mg/kg/day, with individual doses ranging from about 5 mg/kg to about 7 mg/kg for twice daily dosing, or from about 10 mg/kg to about 14 mg/kg for once daily dosing.

In some embodiments, the methods, compositions, and systems of the present invention achieve lower peak blood levels of bucillamine compared to immediate-release formulations (or delayed-release formulations), such as achieving peak blood levels of no more than about 30,000 ng/mL, preferably no more than about 25,000 ng/mL, more preferably no more than about 20,000 ng/mL, and still more preferably no more than about 15,000 ng/mL, e.g., reaching levels of no more than about 25,000 to about 30,000 ng/mL, about 20,000 to about 25,000 ng/mL, about 15,000 to about 20,000 ng/mL, or about 10,000 to about 15,000 ng/mL total bucillamine. In some embodiments, the methods, compositions, and systems of the present invention achieve lower, and more consistently lower, urinary cystine concentrations compared to immediate-release (or delayed-release) formulations, such as keeping urinary cystine effectively below about 250 mg/L for the at least about 8 hours, at least about 10 hours, or preferably for at least about 12 hours of extended duration of effect; preferably effectively below about 200 mg/L for the at least about 8 hours, at least about 10 hours, or for at least about 12 hours of extended duration of effect; more preferably effectively below about 150 mg/L for the at least about 8 hours, at least about 10 hours, or for at least about 12 hours of extended duration of effect; and even more preferably at or effectively below about 100 mg/L for the at least about 8 hours, at least about 10 hours, or for at least about 12 hours of extended duration of effect. In some embodiments, the methods, compositions, and systems of the present invention achieve greater urinary unbound bucillamine recovery compared to immediate-release (or delayed-release) formulations, such as urinary recovery of about 25% to about 75%, about 40% to about 60%, or about 50%, of the administered drug, over about 12 hours of extended effect. The methods, compositions, and systems disclosed herein can thus provide better control of cystine concentration in the urine, including controlling urinary cystine levels overnight, thereby more effectively reducing stone formation for cystinuria patients.

Still another aspect of the invention provides methods of using a pharmaceutical composition or delivery system disclosed herein, in oral or other formulations, to deliver a therapeutically effective amount of bucillamine for achieving an extended duration therapeutic effect in a subject in need thereof, such as a patient with an inflammatory condition, such as gout, rheumatoid arthritis, or a related condition. In some embodiments, for gout, rheumatoid arthritis, or related conditions, the methods, compositions, and systems of the present invention achieve higher and steadier plasma free bucillamine concentrations compared to immediate-release (or delayed-release) formulations, such as plasma free bucillamine concentrations varying from about 500 to about 3,500 ng/mL, about 700 to about 2,500 ng/mL, or about 1,000 to about 2,000 ng/mL, over about 12 hours of extended effect.

For treating or preventing an inflammatory condition, the pharmaceutical composition generally is delivered so as to provide a total daily dose of bucillamine used for that condition. In preferred embodiments, for rheumatoid arthritis and related conditions, the pharmaceutical composition is delivered so as to provide a total daily dose of 300 mg or less, or about 3 mg/kg/day or less; or an average total daily dose of about 300 mg or less, or about 3 mg/kg/day or less, where the dosage indicates the amount of active ingredient (bucillamine), and the bucillamine is released in repeated, critically-spaced pulses, according to the invention, preferably in the stomach. In particular embodiments, the composition comprises bucillamine in a first treatment dose that achieves an extended duration therapeutic effect, lasting at least about 8 hours, preferably before a second or subsequent treatment dose is administered, thereby extending the interval between doses for the patient. In preferred embodiments for rheumatoid arthritis or related conditions, use of the modified-release formulation or system achieves therapeutic effect for a duration of at least about 10 hours, at least about 14 hours, or at least about 18 hours; and/or the treatment dose is administered no more than twice a day. In more preferred embodiments, the total daily dose used is less than about 300 mg/day, such as treatment doses of about 70 mg or less administered twice a day; or, even more preferably, treatment doses of about 40 mg or less administered twice a day.

In preferred embodiments, for gout and related conditions, the pharmaceutical composition is delivered so as to provide a total daily dose of 250 mg or less, or about 3 mg/kg/day or less; or an average total daily dose of about 250 mg or less, or about 3 mg/kg/day or less, where the dosage indicates the amount of active ingredient (bucillamine), and the bucillamine is released in repeated, critically-spaced pulses, according to the invention, preferably in the stomach. In particular embodiments, the composition comprises bucillamine in a first treatment dose that achieves an extended duration therapeutic effect, lasting at least about 8 hours, preferably before a second or subsequent treatment dose is administered, thereby extending the interval between doses for the patient. In preferred embodiments for gout or related conditions, use of the modified-release formulation or system achieves therapeutic effect for a duration of at least about 10 hours, at least about 14 hours, or at least about 18 hours; and/or the treatment dose is administered no more than twice a day. In more preferred embodiments, the total average daily dose used is less than about 200 mg/day, such as treatment doses of about 100 mg or less administered twice a day; or, even more preferably, treatment doses of about 75 mg or less administered twice a day. In some more preferred embodiments, a patient suffering from a gout flare may be able to achieve relief with one administration, e.g., achieving reduced joint pain for the course of atypical flare (3-10 days).

Yet still another aspect of the invention provides methods of using a pharmaceutical composition disclosed herein to achieve a therapeutic effect in a subject in need thereof, such as a patient with cystinuria, a related disorder, gout and/or rheumatoid arthritis, or a condition related to either, where the pharmaceutical composition comprises a pharmaceutically acceptable carrier and bucillamine in a treatment dose for repeated administration to the subject, in about four to about eight hour intervals for cystinuria and related disorders, and in about two to about six hour intervals for inflammatory conditions, including gout, rheumatoid arthritis, and related conditions, to provide a total daily or weekly dose below that typically indicated for the disorder or condition. In some embodiments, the composition is an immediate-release composition for oral administration. In some embodiments, the composition is provided as a transdermal or transmucosal formulation. Generally, for cystinuria and related disorders, the composition is administered no more frequently than every five hours, that is no more than about five times a day, so that the interval is no less than about five hours. In a preferred embodiment, for cystinuria and related disorders, the interval is about six hours and the composition is administered about than four times a day. Generally, for gout, rheumatoid arthritis, and related conditions, the composition is administered no more frequently than every three hours, that is no more than about eight times a day, so that the interval is no less than about three hours. In a preferred embodiment, for gout, rheumatoid arthritis, and related conditions, the interval is about four hours and the composition is administered about than six times a day. In preferred embodiments, the strategy provided herein extends the interval between doses for the cystinuria, gout, or rheumatoid arthritis patient; and/or reduces adverse side effects typical of bucillamine.

Yet another aspect of the invention provides kits for use with the compositions, systems, and methods described herein. In some embodiments, kits provide dosage forms, grouped by doses to be taken at a given time and/or organized to aid compliance with a particular dosing regimen.

### BRIEF DESCRIPTION OF THE DRAWINGS

FIG. 1 depicts representative hypothetical plasma bucillamine levels of patients receiving a modified-release (MR) formulation of bucillamine, in accordance with the present invention, compared with plasma bucillamine levels of patients receiving immediate-release (IR) and delayed-release (DR) bucillamine formulations.

FIG. 2 depicts further details of representative hypothetical plasma bucillamine levels, distinguishing plasma levels of bound and free bucillamine for patients receiving a modified-release formulation (MR bound and MR free, respectively), in accordance with the present invention, compared with plasma levels of bound and free bucillamine for patients receiving immediate-release bucillamine formulations (IR bound and IR free, respectively), where the modified-release formulation increases availability of free bucillamine with lower total dose.

FIG. 3 depicts a representative modified-release component comprising layers of coatings, as seen in a cross-section of a pill or table, having a core of bucillamine (301) surrounded by alternating layers of differently-dissolving coatings (302) and bucillamine (303), where the different layers dissolve at step-wise, critically-spaced times, in the same or different areas of the gastrointestinal tract, to release repeated pulses of bucillamine in accordance with aspects of the invention.

FIG. 4 depicts a representative modified-release component comprising a matrix of differently-dissolving segments, as seen in a cross-section of a pill or table, having multiple reservoirs of bucillamine (401) surrounded by different matrix thicknesses (402), where the different segments dissolve at step-wise, critically-spaced times, in the same or different areas of the gastrointestinal tract, to release repeated pulses of bucillamine in accordance with aspects of the invention.

FIGS. 5A-5B depict a representative modified-release component comprising a capsule of differently-coated beads, as seen in a cross-section of the capsule (501), with multiple bucillamine-containing beads (502) having different coatings (503), where the different coatings dissolve at step-wise, critically-spaced times, in the same or different areas of the gastrointestinal tract, to release repeated pulses of bucillamine in accordance with aspects of the invention.

FIGS. 6A-6B depict a representative modified-release component comprising a capsule of differently-dissolving bands, as seen in a cross-section of the capsule (601), separated into multiple bands (602) by intervening non-dissolving rings (603), to effect dissolution and release (604)-(611), where the different bands dissolve at step-wise, critically-spaced times, in the same or different areas of the gastrointestinal tract, to release repeated pulses of bucillamine in accordance with aspects of the invention.

FIG. 7 depicts a representative modified-release component comprising a capsule with differently-releasing plugs, as seen in a cross-section of the capsule (701), where the different plugs (702) separate multiple compartments of bucillamine (703) and become unplugged at step-wise, critically-spaced times, in the same or different areas of the gastrointestinal tract, to release repeated pulses of bucillamine in accordance with aspects of the invention.

FIGS. 8A-8B depict a representative modified-release component comprising a capsule of packets with differently-dissolving coatings and differently-expanding osmotic-push compartments, as seen in a cross-section of the capsule (801) and packets (802), where the different coatings (803) dissolve at step-wise, critically-spaced times, in the same of different areas of the gastrointestinal tract, and/or and the different osmotic-push compartments (804) expand at step-wise, critically-spaced times, in the same or different areas of the gastrointestinal tract, and details thereof (805)-(811), to release repeated pulses of bucillamine in accordance with aspects of the invention.

FIG. 9 depicts a representative modified-release component comprising a two-layer matrix, one layer comprising drug for immediate-release (901); and one layer comprising drug embedded in a stomach-releasing polymer (902).

FIGS. 10A-10B depict representative modified-release components, each coated by a gastro-retentive stomach-adhering coating. FIG. 10A depicts the coating of stomach-adhering polymers (1001); FIG. 10B depicts adherence of the coating to the stomach lining.

FIGS. 11A-11B depict a representative gastro-retentive sedimentation system for modified-release, comprising immediate release pellets and high-density pellets. FIG. 11A depicts a capsule (1101), shown in cross section, comprising a combination of immediate release bucillamine pellets (1102) and high density bucillamine pellets (1103). FIG. 11B depicts immediate release of bucillamine at a first time point (1) and later at a second time point (2) in accordance with aspects of the invention.

FIGS. 12A-12B depict a representative gastro-retentive expandable system for modified-release, comprising a shape-modifying structure. FIG. 12A depicts administration (1201), unfurling (1202), and retention (1203) of the structure. FIG. 12B depicts use of different “arms” of the structure as differently-releasing matrices, to release drug at step-wise, critically-spaced times in accordance with aspects of the invention.

FIG. 13 depicts a representative gastro-retentive expandable system for modified-release, comprising a swellable structure formed by in-situ gel formation; after ingestion (1301), the structure forms a swollen gel (1302) that remains in the stomach, releasing drug (1303) at step-wise, critically-spaced times in accordance with aspects of the invention.

FIGS. 14A-14B depict representative multiple unit (FIG. 14A) and single unit (FIG. 14B) dosage forms of gastro-retentive floatation systems for modified-release, comprising immediate release bucillamine microbeads and bucillamine microbeads coated in a density-lowering matrix of buoyant polymers. FIG. 14A depicts a multiple unit capsule (1401), shown in cross section, comprising the immediate release bucillamine microbeads (1402) and coated microbeads coated (1403). FIG. 14B depicts a single unit capsule (1404), shown in cross section, comprising an outer layer of immediate release bucillamine (1405), that coats a buoyant polymer matrix (1406), which itself encapsulates a core of bucillamine (1407).

FIG. 15 depicts a representative gastro-retentive floatation system comprising an effervescent agent, where a bead of an expandable polymer (1501) surrounds an NaCO₃ layer (1502), that surrounds bucillamine (1503); the NaCO₃ generates carbon dioxide (1504), buoying up the bead on the stomach fluid surface (1505), where the bead later releases drug (1506).

FIG. 16 depicts a representative modified-release gastro-retentive floatation system, comprising a capsule (1601) of immediate release beads (1602) and beads surrounded by NaCO₃ (1603) and an expandable polymer (1604), that together release bucillamine immediately (1) and after a critical interval of time (2), in accordance with aspects of the invention.

FIGS. 17A-17D depict representative transdermal delivery systems with modified-release components, including a system comprising micro-reservoirs (FIG. 17A), a matrix dispersion (FIG. 17B), a peripheral adhesive (FIG. 17C), and a single reservoir (FIG. 17D).

FIGS. 18A-18B depict additional representative transdermal delivery systems with modified-release components comprising microneedles, including systems with solid, coated, dissolving, or hollow microneedles. FIG. 18A depicts the systems before release; FIG. 18B depicts the systems during and after release of bucillamine.

FIG. 19 depicts total bucillamine concentration (ng/mL) in plasma at different times post-dosing, using an animal model of Beagle dogs orally (PO) administered 30 mg/kg bucillamine according to 4 dosage regimens: a single 30 mg/kg dose (Q12H PO, Leg 1); two 15 mg/kg doses 6 hours apart (Q6H PO, Leg 2); four 7.5 mg/kg doses, every 3 hours (Q3H PO; Leg 3); and six 5 mg/kg doses, every 2 hours (Q2H PO, Leg 4); as well as that following intraduodenal (ID) administration of a single 30 mg/kg dose (Q12H ID, Leg 5). Values are averaged; bars represent standard deviation (n=3 dogs).

FIG. 20 depicts free bucillamine concentration (ng/mL) in plasma at different times post-dosing, using Beagle dogs orally administered 30 mg/kg bucillamine according to 4 dosage regimens a single 30 mg/kg dose (Q12H PO, Leg 1); two 15 mg/kg doses 6 hours apart (Q6H PO, Leg 2); four 7.5 mg/kg doses, every 3 hours (Q3H PO; Leg 3); and six 5 mg/kg doses, every 2 hours (Q2H PO, Leg 4); as well as that following intraduodenal (ID) administration of a single 30 mg/kg dose (Q12H ID, Leg 5). Values are averaged; bars represent standard deviation (n=3 dogs).

FIG. 21 depicts free bucillamine in urine, as a % of dose (unchanged drug), at different times post-dosing, using Beagle dogs orally administered 30 mg/kg bucillamine according to 4 dosage regimens: a single 30 mg/kg dose (Q12H PO, Leg 1); two 15 mg/kg doses 6 hours apart (Q6H PO, Leg 2); four 7.5 mg/kg doses, every 3 hours (Q3H PO; Leg 3); and six 5 mg/kg doses, every 2 hours (Q2H PO, Leg 4); as well as that following an intraduodenal (ID) administration of a single 30 mg/kg dose (Q12H ID, Leg 5). Values are averaged; bars represent standard deviation (n=3 dogs).

FIG. 22 depicts free bucillamine urine recovery per time frames: 0-4 hours, 4-8 hours, 8-12 hours, and 12-24 hours, for a single 30 mg/kg dose (Q12H PO, Leg 1) and two 15 mg/kg doses 6 hours apart (Q6H PO, Leg 2). For this analysis, only dogs that produced a urine sample for each time frame were considered as a result, and two dogs per group were used. Values are averaged in % of total drug dose urine recovery per dosing group. Bars represent standard deviation (n=2 dogs).

Other aspects, features and advantages of the invention will become apparent from the following detailed description and illustrative examples.

### DETAILED DESCRIPTION

The present invention provides methods, compositions, devices, and kits for use in administration of bucillamine in modified-release formulations to treat metabolic disorders of the liver and kidneys, in particular to treat or prevent cystinuria, disorders related thereto, and symptoms thereof. Currently approved cystinuria medications result in high peak plasma levels and rapid clearance in the urine, leading to adverse side effects and the need for frequent dosing. Immediate-release bucillamine, currently in clinical trials, may pose similar challenges. The compositions, kits, devices, and methods of the present invention provide bucillamine in critically-spaced, repeated pulses over time, to the gastrointestinal tract and/or the bloodstream, preferably in the stomach, to achieve efficacy with lower peak plasma levels, but consistently higher urinary levels of the unbound drug, thereby allowing less frequent and/or lower total dosages, and leading to increased patient compliance and improved quality of life. The modified-release strategy similarly affords higher efficacy with higher and steadier plasma levels of unbound drug in the treatment of other conditions where bucillamine is indicated, such as gout and/or rheumatoid arthritis, thereby similarly allowing less frequent and/or lower total dosages, increased patient compliance and improved quality of life.

Cystinuria is an autosomal recessive genetic disease that is characterized by high concentration of the amino acid cysteine in the urine, leading to formation of cystine stones in the kidneys, ureter, and bladder. Mutations in either the SLC3A1 or SLC7A9 gene cause cystinuria, with the disease manifesting in patients with two copies of one of the defective genes. Due to the gene defects, a pump in the brush border membrane of the proximal renal tubules fails to reabsorb cysteine filtered out of the blood, as well as failing to absorb lysine, ornithine, and arginine (Eggermann et al., 2012, “Cystinuria: an inborn cause of urolithiasis” Orphanet J Rare Dis. 7:19). In particular, accumulation of cysteine leads to abnormally high levels in the urinary tract, and subsequent formation and precipitation of cystine (the oxidized dimer of cysteine) as debilitating stones in the kidneys (nephrolithiasis) and ureters (ureterolithiasis). The disorder has a profoundly negative impact on the quality of life, causing excruciating pain as patients attempt to pass the frequent-forming and sometimes very large stones. Further, cystine stones are often difficult to fragment non-invasively, e.g., using non-invasive extracoporal lithothipsy. Rather, more invasive percutaneous nephrostomy often is required for stone removal. In severe cases, the patient's urine has the consistency of sand and the kidneys and surrounding organs become damaged, even to point of requiring renal transplant.

Cystinuria is a rare disease, affecting about 30,000 patients in the U.S. and about 1 in 7,000 worldwide (Biyani, et al., 2015, “Cystinuria Diagnosis and Management,” EAU-EBU Update Series, 4(5):175-183). Among adults, cystine stones account for about 1-2% of urinary nephrolithiasis patients; in children, 6-8% of urinary nephrolithiasis patients suffer from cystine stones (Stapleton et al., 1987, “Urolithiasis in children: the role of hypercalciuria,” Pediatr Ann. 16(12):980-981; 984-992). Of patients carrying two SLC3A1 mutations, two SLC7A9 mutations, or one copy of each mutation, more than 95% develop stones (Eggermann et al., 2012, “Cystinuria: an inborn cause of urolithiasis” Orphanet J Rare Dis. 7:19). Further, 62 out of 106 cystinuria patients are affected by recurrent cystine urolithiasis (Linari et al., 1981, “The natural history of cystinuria: a 15 year follow-up in 106 patients” Urolithiasis: Clinical and Basic Research, Ed. L H Smith, Springer Science & Business Media). Cystinuria varies in severity, as scored primarily by frequency of stone formation. Nonetheless, at least half of all cases (or about 15,000 individuals in the U.S.) require daily medication for the rest of their lives. Cystinuria also is a concern for dog owners, being a common disorder in certain dog breeds, in particular, Bulldogs, Newfoundlands, and Labrador Retrievers (Hoppe, 2001, “Cystinuria in the dog: clinical studies during 14 years of medical treatment” J Vet Intern Med 15(4): 361-367). Indeed, cystinuria is estimated to affect a considerable percentage of pet dogs in the U.S.

Treatment options aim to reduce cystine concentration in the kidneys and to increase its solubility, so as to reduce stone formation. Generally, the aim is to keep urinary cystine concentrations below 1,200 μmol/L (Lindell et al., “Clinical Course and Cystine Stone Formation During Tiopronin Treatment,” Urol. Res., 23, pp. 111-117, 1995); that is, below 250 mg/L. At or above this concentration, stone formation begins and, once started, is irreversible.

Options include hydration, alkalization, and use of chelating agents. In terms of hydration, patients are told to drink 4-6 L of water per day, that is, drinking 240 mL of water every hour during the day and 480 mL before retiring for the night. The large quantities of water serve to reduce cystine concentration accumulating in urine. Regarding alkalization, the pH and salinity of urine can be altered to keep cystine soluble, reducing stone formation. To this end, patients are advised to reduce salt intake and/or to take potassium citrate supplements to raise the pH of urine.

Chelating agents are used to change the type of cystine to a more soluble form. Specifically, cystine can react with a thiol-binding compound (RS) to give cysteine and a mixed thiol, R-cysteine, both of which are more soluble in urine than cystine. First-generation medications for cystinuria used D-penicillamine that forms a di-sulfide complex 50 times more soluble than cystine. Bioavailability of D-penicillamine, however, dramatically decreases in patients with malabsorption states, or in the presence of antacids, or even after a large meal (see, Bergstrom et al., 1981, “Penicillamine Kinetics in Normal Subjects,” Penicillamine Kinetics, 30(2):404-413; Ifan et al., 1986, “Short Communication, Pharmacokinetics of Oral 500-MG Penicillamine: Effect of Antacids on Absorption,” Biopharnaceutics & Drug Disposition, 7:401-405; and Netter et al., 1987, “Clinical Pharmacokinetics of D-Penicillamine,” Clinical Pharmacokinetics 13:317-333). Further, D-penicillamine has been associated with serious side effects, including rashes, arthralgia, leukopenia, gastrointestinal intolerance, nephritic syndrome, and vitamin B6 deficiency. Indeed, severe side effects in as many as 50-70% of patients limit the use of D-penicillamine.

In 1988, the FDA approved the use of tiopronin, alpha-mercaptopropionylglycine (alpha-MPG), as a second-generation chelating agent (sold under the tradename THIOLA®). Tiopronin is considered a hepatoprotective and anti-cataract agent, as well as being used in treating rheumatoid arthritis in Europe, where it is marketed as Acadione® tablets. For cystinuria, in the United States, the drug currently is marketed by Retrophin in an immediate-release formulation (Thiola®), as 100 mg tablets of a racemic mixture of the (+) and (−) enantiomers of tiopronin. Prescribed average doses are 1,200 mg/day, taken in 400 mg doses, 3 times in a 24 hour period. Immediate-release tiopronin has two urine excretion waves, the primary one occurring 3-4 hours after oral administration (see, e.g., Hercelin et al., “The Pharmacokinetics of Tiopronin and its Principal Metabolite (2-mercaptopropionic acid) After Oral Administration to Healthy Volunteers,” Eur. J. Clin. Pharmacol., 43, pp. 93-95, 1992). Initial doses may start at 800 mg/day and adjusted upwards until an effective dose is determined for an individual patient. A typical recommended dose is 10 mg/kg/day for children and adults, which can be increased to, e.g., about 30 mg/kg/day for adults in severe cases of cystinuria. Urine excretion of free tiopronin and mixed-sulfide tiopronin mostly occurs 2-4 hours after administration and as much as 95% of free tiopronin is excreted in urine within 2 hours of Thiola® administration (see, e.g., Carlsson et al., 1993, “Pharmacokinetics of oral tiopronin,” European Journal of Clinical Pharmacology 45:79-84; and Carlsson et al., 1994, “Pharmacokinetics of 2-Mercaptopropionylglycine (Tiopronin) in Patients with Impaired Renal Function,” Drug Invest. 7(2):101-112). Nonetheless, Thiola® is discontinued in about 30% of cases (Pak et al., 1986, Management of cystine nephrolithiasis with alpha-mercaptopropionylglycine, J. Urol. 136(5): 1003-1008), due to adverse reactions, the need for frequent dosing, and poor efficacy often due to uncontrolled cystine levels at night. That is, Thiola® is only effective in reducing cystine urine levels for short periods of time, not exceeding 2-4 hours, and thus requires frequent administration. Accordingly, the drug fails to address rising cystine levels during the night, between evening and morning doses, resulting in cystine crystals precipitating overnight, to form “seeds” that are irreversible and eventually grow to cystine stones. Further, when taken at higher doses, Thiola® can lead to even more serious adverse effects, with increased incidence, leading to poor compliance. Indeed, one study determined that only 15% of patients achieve and maintain therapeutic success with Thiola® (Pietrow et al., 2003, “Durability of the medical management of cystinuria” J Urol. 169(1):68-70). Delayed-release forms of tiopronin or bucillamine may fail to address these issues, as merely delaying the release of drug under the same pharmacokinetic profile, rather than providing extended duration of availability of unbound drug in the blood and in the urine, with lower peak levels of total drug in the blood.

Bucillamine is a potential new therapeutic agent for cystinuria. Bucillamine, or N-(2-mercapto-2-methylpropionyl)-L-cysteine (IUPAC name (2R)-2-[(2-methyl-2-sulfanyl-propanoyl) amino]-3-sulfanylpropanoic acid, is a di-thiol compound containing two donatable thiol groups, compared to D-penicillamine or tiopronin, which each contain only one thiol group. Bucillamine results in a cysteine-bucillamine complex for removing excess cysteine from the urine and has been shown to dissolve cystine in vitro (Biyani, et al., 2006, “Cystinuria diagnosis and management,” EAU-EBU Update Series, 4:175-183; and U.S. Pat. No. 5,266,595 to Baba et al. (Santen Pharmaceutical Co., Ltd.)). Bucillamine is manufactured in Japan by Santen Pharmaceutical Co. Ltd. as Rimatil®, which is used as a first-line treatment for rheumatoid arthritis in Japan and South Korea. More recently, bucillamine has been studied for treating gout. A Phase 2 study has been completed to assess efficacy and safety of bucillamine in subjects with moderate to severe gout (https://clinicaltrials.gov/ct2/show/NCT02330796). For cystinuria, bucillamine is undergoing clinical trials, in a Phase 2 study for cystinuria patients failing therapy with tiopronin (https://clinicaltrials.gov/ct2/show/NCT02942420). In the trials, subjects will be administered bucillamine tablets orally, three times a day, around mealtime. The bucillamine tablets being used provide an immediate-release formulation of bucillamine. The study envisions dosing subjects in a sequential manner, with a first group receiving one 100 mg bucillamine tablet three times a day; followed by a second group receiving two 100 mg bucillamine tablet three times a day. Accordingly, the study fails to address the problem of requiring frequent dosing, with resultant poor compliance and possible poor efficacy due to uncontrolled cystine levels between doses, especially overnight. As in the case of tiopronin, the drug would fail to address rising cystine levels during the night, between evening and morning doses, that results in cystine crystals precipitating overnight, to form “seeds” that are irreversible and eventually grow to cystine stones. Delayed-release forms may fail to address these issues as well, as merely delaying the release of bucillamine, rather than providing extended duration of lower levels of the drug over longer time periods.

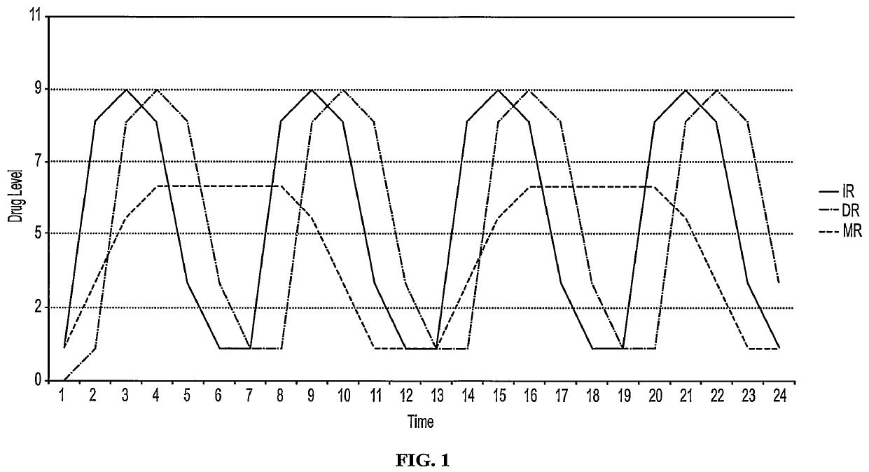

Strategies described herein provide benefits and advantages over other compositions and methods in which bucillamine is administered in immediate-release formulations, as well as advantages over compositions and methods using delayed-release formulations. FIG. 1, for example, depicts representative hypothetical results of plasma bucillamine levels of patients receiving a modified-release (MR) formulation of bucillamine, in accordance with the present invention, compared with plasma bucillamine levels of patients receiving immediate-release (IR) and delayed-release (DR) bucillamine formulations. As FIG. 1 illustrates, use of a delayed-release formulation may provide peak plasma levels similar to those using an immediate-release formulation; however, use of a modified-release formulation can dramatically decrease peak plasma levels of the drug.

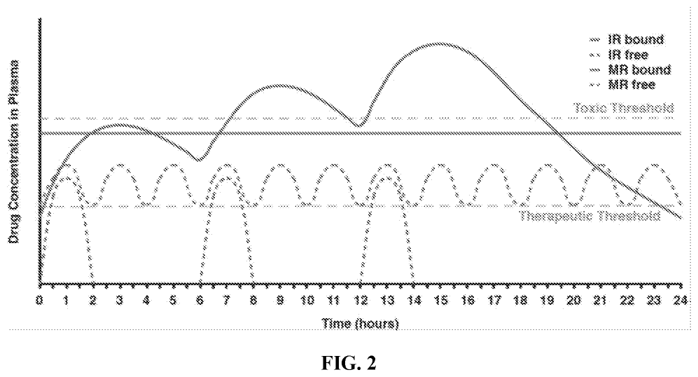

FIG. 2 depicts further details of representative hypothetical plasma bucillamine levels, distinguishing plasma levels of bound and free bucillamine for patients receiving a modified-release formulation (MR bound and MR free, respectively), in accordance with the present invention, compared with plasma levels of bound and free bucillamine for patients receiving immediate-release bucillamine formulations three times a day (IR bound and IR free, respectively), where the modified-release formulation increases availability of free bucillamine with lower total dose or lower total drug plasma peaks. As used herein, “free bucillamine” or “free drug” is used interchangeably with “unbound bucillamine” or “unbound drug” and refers to drug that is not bound to protein, e.g., not bound to protein in the plasma or in the urine. The sum of free drug and bound drug amounts, gives the total amount of drug in the plasma or urine. Since there typically are no proteins in urine, urinary free drug coincides with urinary total drug. As FIG. 2 illustrates, use of the modified-release formulation lowers and more consistently maintains levels of bound bucillamine in the plasma (compare the solid lines), e.g., maintaining plasma levels below a “toxic threshold,” at or above which increased side effects occur. The modified-release formulation also increases and more consistently maintains free bucillamine in the plasma (compare the dotted lines), e.g., maintaining plasma levels above a “therapeutic threshold,” e.g., below which increased stone formation resumes for cystinuria patients, or below which efficacy wanes in treating gout or rheumatoid arthritis.

The present invention provides methods, compositions, devices, and kits for administering bucillamine according to a modified-release strategy to provide lower peak plasma levels, but consistently higher urinary levels of the drug, where it acts to reduce cystine concentrations and stone formation in conditions like cystinuria. “Consistently higher” urinary levels of the drug encompass situations where urinary levels are maintained at desired levels for longer periods of time compared to the same or similar desired levels of an immediate-release (or a delayed-release) version of the drug, e.g., providing urinary bucillamine recovery of about 25-75% of the total dose administered, for at least about 8-12 hours of extended duration of effect. Use of the present modified-release compositions or systems reduces side effects of bucillamine and enhances efficacy, allowing for less frequent administrations and/or lower total daily dosage, thereby improving patient compliance and overall quality of life.

The present invention also provides methods, compositions, devices, and kits for administering bucillamine according to a different modified-release strategy to provide lower peak plasma levels, but higher and steadier plasma levels of the unbound drug, for use in conditions like arthritis or gout. “Steadier” plasma levels of the free drug encompass situations where plasma levels are maintained at desired levels for longer periods of time compared to the same or similar desired levels of an immediate-release (or a delayed-release) version of the drug, e.g., providing therapeutically effective plasma levels for at least about 8-12 hours of extended duration of effect for gout or rheumatoid arthritis patients, again reducing side effects, enhancing efficacy, and allowing for less frequent administrations and/or lower average total daily (or weekly) doses.

Modified-Release Compositions and Systems

One aspect of the present invention provides compositions and systems (or articles of manufacture) for use in effecting modified-release of a therapeutic agent. Generally, the invention provides a pharmaceutical composition or other delivery system comprising the therapeutic agent and a pharmaceutically acceptable modified-release component for releasing the therapeutic in repeated, critically-spaced pulses over time, following administration or application of the composition or system to a subject. A “pulse” or “periodic pulse” can refer to a distinct bolus of drug delivered or released repeatedly over time, where less drug, preferably substantially less drug, or more preferably almost no drug, is delivered or released between consecutive pulses during a critical interval of time. A “bolus” of a drug refers to a discrete amount of the drug released within a specific time, e.g., within about 1 second to about 30 minutes, preferably about 30 seconds to about 20 minutes, more preferably about 1 to about 15 minutes.

In particular embodiments, the therapeutic agent comprises bucillamine. The bucillamine may comprise a racemic or non-racemic mixture of bucillamine enantiomers; or may comprise substantially one or other of the (+) or (−) enantiomer of bucillamine. The structure of bucillamine is provided below, as Formula I. The structures of the (+) and (−) enantiomers are shown below, in Formula IIa and b, respectively.

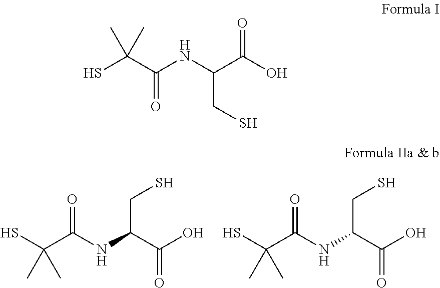

This disclosure is not limited with respect to bucillamine. The bucillamine may be a racemic mixture of bucillamine enantiomers or bucillamine derivatives; or a non-racemic mixture of bucillamine enantiomers or bucillamine derivatives, as well as substantially 100% of one or other specific bucillamine enantiomer or derivative thereof. Each bucillamine enantiomer ((2R)-2-[(2-methyl-2-sulfanylpropanoyl)amino]-3-sulfanylpropanoic acid and (2S)-2-[(2-methyl-2-sulfanylpropanoyl)amino]-3-sulfanylpropanoic acid), may have different pharmacokinetic parameters. A “non-racemic mixture” of bucillamine enantiomers refers to a mixture having over 50% of one or other of the two enantiomers. In some embodiments, the non-racemic mixture contains at least about 55%, at least about 60%, at least about 70%, at least about 75%, at least about 80%, at least about 85%, at least about 90%, at least about 95%, at least about 98%, or more, or about 100% of the (+) enantiomer compared to the (−) enantiomer. In some embodiments, the non-racemic mixture contains at least about 55%, at least about 60%, at least about 70%, at least about 75%, at least about 80%, at least about 85%, at least about 90%, at least about 95%, at least about 98%, or more, or about 100% of the (−) enantiomer compared to the (+) enantiomer. In a particular embodiment, the bucillamine comprises a (+) enantiomer of bucillamine and substantially no (−) bucillamine enantiomer; or comprises a (−) enantiomer of bucillamine and substantially no (+) bucillamine enantiomer. In some embodiments, a racemic mixture is provided, e.g., comprising about 50% of each enantiomer ((2R,S)-2-[(2-methyl-2-sulfanylpropanoyl)amino]-3-sulfanylpropanoic acid).

In one aspect, the modified-release component of the present invention provides pulses of the therapeutic agent, such as bucillamine, released to the subject at critically-spaced intervals, affording lower but more consistently-maintained plasma levels of the drug, as well as better urine recovery, that is, higher and more consistently-maintained urinary levels of the drug, compared to approaches with longer or shorter intervals between doses. Consistently higher bucillamine levels in urine result in lower and more consistently-maintained urinary cystine concentrations, compared to approaches with longer or shorter intervals between doses, thereby increasing efficacy in treating cystinuria or a related disorder. A “more consistently-maintained” or “more consistently lower” free (unbound) bucillamine level or concentration in the blood or plasma, means that preferred concentrations of free bucillamine in the blood or plasma vary within a range of about 300 to about 3,000 ng/mL, about 500 to about 2,500 ng/mL, about 500 to about 2,000 ng/mL, about 700 to about 2,000 ng/mL, about 700 to about 1,500 ng/mL, or about 1,000 to about 1,500 ng/mL, for an extended period of therapeutic effect.

In another aspect, the modified-release component of the present invention provides more closely-spaced pulses of the therapeutic agent, such as bucillamine, released to the subject at shorter, critically-spaced intervals, affording lower peak, but higher and more consistently-maintained (or steadier) plasma levels of unbound drug, compared to approaches with longer or shorter intervals between doses. Such a pharmacokinetic profile improves efficacy, while reducing side effects and affording fewer administrations, in treating gout, rheumatoid arthritis, or a related condition, where the drug's efficacy depends more on free drug levels in the plasma rather than levels in the urine. A “steadier” or “more consistently higher” free (unbound) bucillamine level or concentration in the blood or plasma, means that preferred concentrations of free bucillamine in the blood or plasma vary within a range of about 500 to about 3,500 ng/mL, about 500 to about 3,000 ng/mL, about 700 to about 3.00 ng/mL, about 700 to about 2,500 ng/mL, or about 1,000 to about 2,000 ng/mL, for an extended period of therapeutic effect.

The pulses provide fractions of a total therapeutically effective amount of drug over a given period of time. It is generally believed that splitting a total dose into multiple smaller doses, individually administered over time, produces higher drug recovery in the urine as compared to giving a single large dose (Lindell et al., 1995, “Urinary excretion of free cystine and the tiopronin-cysteine-mixed disulfide during long term tiopronin treatment of cystinuria” Nephron 71(3):328-342; see also, e.g., Koide et al., 1994, “A new therapeutic agent for cystinuria,” Urolithiasis 2:571-574), and further that with each smaller dose, delivered with increasing frequency, recovery in the urine increases further. Surprisingly, however, it has been found that this relationship for bucillamine is not linear, but bell-shaped. That is, for a given total dose for a given period of time, higher urine recovery of bucillamine occurs over a critical range of dosing frequency or interval length during that time period, whereas more and more frequent dosing beyond this window unexpectedly returns urine recovery to the lower levels expected with less frequent dosing. For example, for a given total daily dose, higher bucillamine urine recovery occurs when the total dose is delivered in fractions of that dose at intervals varying in length within a critical range, whereas using shorter and shorter intervals outside this window unexpectedly returns bucillamine urine recovery to the lower levels expected when using longer intervals.

In some embodiments relating to treating cystinuria or cystinuria related disorders, the modified-release component provides an interval between two consecutive pulses of about four to about eight hours, preferably about five to about seven hours, or more preferably about six hours apart. In some embodiments, the interval between two consecutive pulses is 3 hours, 3.5 hours, 4 hours, 4.5 hours, 5 hours, 5.5 hours, 6 hours, 6.5 hours, 7 hours, 7.5 hours, 8 hours, 8.5 hours, or 9 hours. In more preferred embodiments, the interval between two consecutive pulses is about 5 to about 7 hours, about 5.2 to about 6.8 hours, about 5.4 to about 6.6 hours, about 5.6 to about 6.4 hours, about 5.8 to about 6.2 hours, about 5.9 to about 6.1 hours, or about 6 hours.

It also generally is believed that splitting a total dose into multiple smaller doses, individually administered over time, produces lower peak plasma levels but steadier plasma concentrations over time, as compared to giving a single large dose, and that with each smaller dose, delivered with increasing frequency, this trend would increase further and further. Surprisingly, however, it has been found that this relationship for free (unbound) bucillamine also is not linear, but bell-shaped. That is, for a given total dose for a given period of time, higher and steadier plasma concentrations of free bucillamine occur over a critical range of dosing frequency or interval length during that time period, whereas more and more frequent dosing beyond this window unexpectedly results in lower and less steady plasma levels of the unbound drug, normally associated with less frequent dosing. For example, for a given total daily dose, highest and steadiest free bucillamine plasma levels occur when the total dose is delivered in fractions of that dose at intervals varying in length within a critical range, whereas using shorter and shorter intervals outside this window unexpectedly returns free bucillamine plasma levels and fluctuations to those expected when using longer intervals.

In some embodiments relating to treating arthritis, gout, or related conditions, the modified-release component provides an interval between two consecutive pulses of about two to about six hours, preferably about three to about five hours, or more preferably about four hours apart. In some embodiments, the interval between two consecutive pulses is 1 hour, 1.5 hours, 2 hours, 2.5 hours, 3 hours, 3.5 hours, 4 hours, 4.5 hours, 5 hours, 5.5 hours, 6 hours, 6.5 hours, or 7 hours. In more preferred embodiments, the interval between two consecutive pulses is about 3 to about 5 hours, about 3.2 to about 4.8 hours, about 3.4 to about 4.6 hours, about 3.6 to about 4.4 hours, about 3.8 to about 4.2 hours, about 3.9 to about 4.1 hours, or about 4 hours.

In some embodiments, the modified-release component provides consecutive pulses that are regularly or approximately regularly spaced. That is, intervals between consecutive pulses may be the same or approximately the same, e.g., where the interval between the first and second pulse is about the same as the interval between the second and third pulse. In some embodiments, the intervals between consecutive pulses vary, e.g., where the interval between the first and second pulse is longer or shorter than the interval between the second and third pulse. In some embodiments, the intervals between consecutive pulses vary, e.g., where the interval between the first and second pulse is longer or shorter than, or the same as, the interval between the second and third pulse; and the interval between the second and third pulse is longer or shorter than, or the same as, the interval between the third and fourth pulse.

In some embodiments, the modified-release component provides pulses of equal or approximately equal fractions of a total therapeutically effective amount of drug over a given period of time. “Approximately equal” generally means that the doses are within 0.01%, 0.05%, or 0.1% of a given amount. For example, for a composition comprising a total daily therapeutically effective amount of bucillamine for cystinuria patients, or patients with a related disorder, the drug may be released in two about equal fractions, spaced about twelve hours apart; or in three about equal fractions, spaced about eight hours apart, in four about equal fractions, spaced about six hours apart; or in five about equal fractions, spaced about five hours apart; or in six about equal fractions, spaced about four hours apart, or in eight about equal fractions, spaced about three hours apart. In a particularly preferred embodiment, the pharmaceutical composition, or other delivery system, comprises a total daily therapeutically effective amount of bucillamine for cystinuria patients, released in four about equal fractions, spaced about six hours apart, providing higher total or unbound bucillamine urinary recovery compared to more rare or more frequent dosing. As another example, for a composition comprising a total daily therapeutically effective amount of bucillamine for rheumatoid arthritis patients, the drug may be released in four about equal fractions, spaced about six hours apart; or in five about equal fractions, spaced about five hours apart; or in six about equal fractions, spaced about four hours apart; or in eight about equal fractions, spaced about three hours apart; or in twelve about equal fractions, spaced about two hours apart. In a particularly preferred embodiment, the pharmaceutical composition, or other delivery system, comprises a total daily therapeutically effective amount of bucillamine, released in six about equal fractions, spaced about four hours apart, providing higher and steadier free bucillamine levels compared to more rare or more frequent dosing. In some embodiments, the modified-release component provides pulses of unequal fractions of a total therapeutically effective amount of drug over a given period of time. In some embodiments, the modified-release component provides pulses of unequal, equal, or approximately equal, fractions of a total therapeutically effective amount of drug over a given period of time.

The modified-release component of the present invention is designed to release at least two pulses of drug, such as bucillamine. The number of pulses generally depends on the total amount of the drug used in the pharmaceutical composition or delivery system comprising the modified-release component and/or how often the composition or system is to be administered or applied to the subject. For example, in some embodiments, a first fraction is released at a first time point and a second fraction is released at a second time point after the first time point. Preferably, the modified-release component allows for more than two pulses of bucillamine, e.g., where a subsequent (n^{th}) fraction is released at a subsequent (n^{th}) time point after a preceding (n−1^{th}) time point. In some embodiments, a third faction is released at a third time point after the second time point; more preferably a fourth, fifth, and sixth fraction is released at a fourth, fifth, and sixth time point after the third, fourth, and fifth time point, respectively. The modified-release component is designed to space consecutive pulses according to intervals described herein.

In some embodiments, for example, in the context of treating cystinuria or related disorder, the modified-release component provides bucillamine in repeated pulses over a time period of about eight to about 48 hours, such as a period of about 12 hours, about 18 hours, about 24 hours, or about 36 hours. In some embodiments, the pharmaceutical composition or delivery system with the modified-release component comprises half a daily dose of bucillamine, fractions of which are delivered in repeated pulses about every four hours, five hours, six hours, seven hours, or eight hours for about 12 hours. In preferred embodiments, the pharmaceutical composition or delivery system comprises a daily dose of bucillamine, fractions of which are delivered in repeated pulses about every four hours, five hours, six hours, seven hours, or eight hours for about 24 hours.

In some embodiments, for example, in the context of treating or preventing an inflammatory condition, like gout or rheumatoid arthritis, related conditions or a symptom thereof, the modified-release component provides bucillamine in repeated pulses over a time period of about eight to about 48 hours, such as a period of about 12 hours, about 18 hours, about 24 hours, or about 36 hours. In some embodiments, the pharmaceutical composition or delivery system with the modified-release component comprises half a daily dose of bucillamine, fractions of which are delivered in repeated pulses about every two hours, three hours, four hours, five hours, or six hours for about 12 hours. In preferred embodiments, the pharmaceutical composition or delivery system comprises a daily dose of bucillamine, fractions of which are delivered in repeated pulses about every two hours, three hours, four hours, five hours, or six hours for about 24 hours.

In some embodiments, for example, in the context of treating or preventing an inflammatory condition, like gout, related conditions or a symptom thereof, the modified-release component provides bucillamine in repeated pulses over a time period of about one day to about two weeks, such as a period of about two days, about four days, about one week, or about one and a half weeks. In some embodiments, the pharmaceutical composition or delivery system with the modified-release component comprises twice an average daily dose of bucillamine, fractions of which are delivered in repeated pulses about every two hours, three hours, four hours, five hours, or six hours for about two days. In preferred embodiments, the pharmaceutical composition or delivery system comprises an average weekly dose of bucillamine, fractions of which are delivered in repeated pulses about every two hours, three hours, four hours, five hours, or six hours for about a week.

Oral Delivery

In one approach, the pharmaceutical composition is formulated for oral delivery. The oral formulations generally comprise a therapeutically effective amount of bucillamine for oral delivery and a pharmaceutically acceptable modified-release component to achieve pulsed release of the therapeutic, in accordance with the present disclosures, upon passage through the gastrointestinal tract. One of skill in the art will envision various pharmaceutical formulation designs and/or arrangements that bring about the desired release schedule, including but not limited to, modified-release components comprising coatings, matrixes, beads, polymers, plugs, osmotic-push compartments, bioadhesives, timed devices, and the like, and any combinations thereof. In preferred embodiments, the modified-release component releases multiple pulses of the therapeutic agent in the stomach. In some embodiments, the modified-release component releases a first amount of the therapeutic agent when the composition reaches the stomach, a second amount of the therapeutic agent as the composition travels through the duodenum, a third amount of the therapeutic agent as the composition travels through the jejunum, a fourth amount of the therapeutic agent as the composition travels through the ileum; and/or a fifth amount of the therapeutic agent as the composition travels through the colon. In some embodiments, more, all, or none of the pulsed releases occur in the stomach; more, all, or none of the pulsed releases occur in the small intestine; more, all, or none of the pulsed releases occur in the duodenum; more, all, or none of the pulsed releases occur in the jejunum; more, all, or none of the pulsed releases occur in the ileum; and/or more, all, or none of the pulsed releases occur in the colon. In preferred embodiments, most or all of the pulsed releases occur in the stomach.

In some embodiments, the modified-release formulation comprises a therapeutic, e.g., bucillamine, contained within matrixes, liposomes, vesicles, microcapsules, microspheres, and the like, or within a solid particulate material, all of which is selected and/or constructed to provide release of the therapeutic over time, in a step-wise fashion in critically-spaced pulses, e.g., as described herein. For example, in a particular embodiment, microbeads of bucillamine are coated and the coated beads contained within a capsule, for release over time in a step-wise fashion in critically-spaced pulses, e.g., as described herein. In some embodiments, the drug is embedded in a waxy matrix, which releases the drug over time in a step-wise fashion in critically-spaced pulses, and the matrix subsequently is excreted in the patient's feces.

In some embodiments, the modified-release component comprises layers of coatings, e.g., arranged in a pill or tablet, where the different coatings dissolve at step-wise, critically-spaced times, to release repeated pulses of bucillamine, e.g., in the same or different areas of the gastrointestinal tract. For example, the layers of coatings may include a coating that dissolves or disintegrates when exposed to conditions in the stomach (a “stomach-dissolving coating”), and/or one or more coatings that dissolve at different times in the stomach or along different parts of the gastrointestinal tract, such as a duodenum-dissolving coating, a jejunum-dissolving coating, an ileum-dissolving coating, and/or a colon-dissolving coating. The coatings are designed to dissolve or disintegrate in respective locations in the gastrointestinal tract using, e.g., pH-dependent delivery, time-controlled delivery, microbially-targeted delivery, polysaccharide based-delivery, or other technology described herein, known in the art (see, e.g., Madhu, et al., 2011, “Colon Specific Delivery System: The Local Drug Targeting” Review Article, International Research Journal of Pharmacy, 2(12): 103-107), and/or to be developed especially in view of the present disclosures. In preferred embodiments, the multiple coatings dissolve in the stomach, at critically-spaced times.

As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock.

In preferred embodiments, the coatings dissolve sequentially over time, for example, to release an n^{th} fraction of the bucillamine at an n^{th} time point after a preceding (n−1)^{th} time point, at critically-spaced intervals as described herein. One approach uses an arrangement resembling a “jawbreaker,” with a core and alternating layers of targeted coatings and drug. The coatings may be of similar or different composition, depending on the physiological environment in the area of the body that will dissolve the coating and trigger the release of drug, and/or may vary in thickness to help achieve release of the drug in repeated, critically-spaced pulses, as described herein, in a given location, such as the stomach.

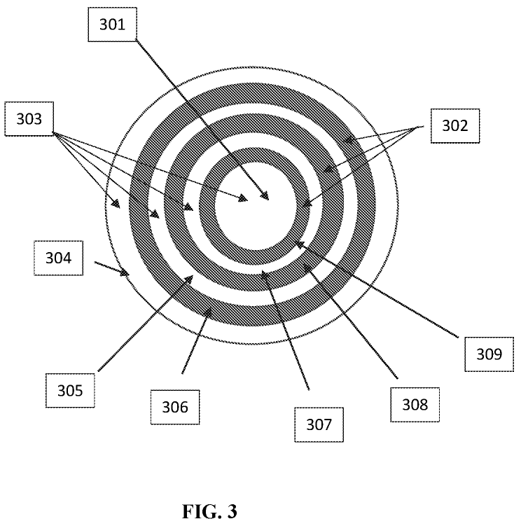

FIG. 3 depicts one example of a modified-release component comprising layers of coatings, as seen in a cross-section of a pill or table, having a core of bucillamine (301) surrounded by alternating layers of differently-dissolving coatings (302) and layers of bucillamine (303), where the different layers are arranged in roughly concentric spheres and dissolve in at step-wise, critically-spaced times to release repeated pulses of bucillamine in accordance with aspects of the invention, the same or different areas of the gastrointestinal tract. In this example, the outermost layer of bucillamine (304) is released in the stomach, the next layer (305) is released in the duodenum upon dissolution of a duodenum-dissolving coating (306); the next innermost layer (307) is released in the jejunum and/or ileum upon dissolution of a jejunum- and ileum-dissolving coating (308), and finally the core (301) is released in the large intestines upon dissolution of a colon-dissolving coating (309). One of skill in the art will readily envision variations of this arrangement in view of the present teachings, e.g., where the core comprises an inert material coated in a layer of bucillamine, followed by alternating layers of differently-dissolving coatings and bucillamine. In some embodiments, a “small-intestine-dissolving coating” may be used instead of, or with, the duodenum-, jejunum-, and/or ileum-dissolving coatings, where the small-intestine-dissolving coating dissolves or disintegrates to release repeated, critically-spaced pulses of drug over time in the small intestines, preferably independent of its position along the small intestines. Further, in some embodiments, additional layers may be used to achieve additional pulses in accordance with the invention.

The different coatings may be arranged so as to provide immediate and subsequent repeated release of the therapeutic. A mixture of types of tablets also can be used, e.g., a first set that releases first and second critically-spaced pulses of bucillamine, e.g., in the stomach and duodenum, respectively; and a second set that can be administered at about the same time as the first set and that releases still later and also critically-spaced third and fourth pulses of bucillamine, e.g., in the jejunum/ileum and in the large intestines, respectively. In a preferred embodiment, the first and second sets are color-coded, differently-labelled, and/or differently-marked, e.g., to allow easy identification by the patient, pet-owner, and/or health practitioner. In some embodiments, a third set is used that can be administered at about the same time as the first and second sets, which releases still later and also critically-spaced fifth and sixth pulses of bucillamine. Preferably, the third set also is color-coded, differently-labelled, and/or differently-marked compared to the first and second sets.

In some embodiments, the modified-release component comprises a matrix of differently-dissolving segments, where the different segments dissolve at step-wise, critically-spaced times, to release repeated pulses of bucillamine, in accordance with aspects of the invention, e.g., in the same or different areas of the gastrointestinal tract. For example, a pill/tablet with drug reservoirs that are exposed sequentially in intervals may be used, such as where the reservoirs are enclosed in segments of different thicknesses and/or segments made up of different types of polymers, selected to disintegrate at different times or in different locations along the gastrointestinal tract. In preferred embodiments, the multiple segments dissolve in the stomach, at critically-spaced times.

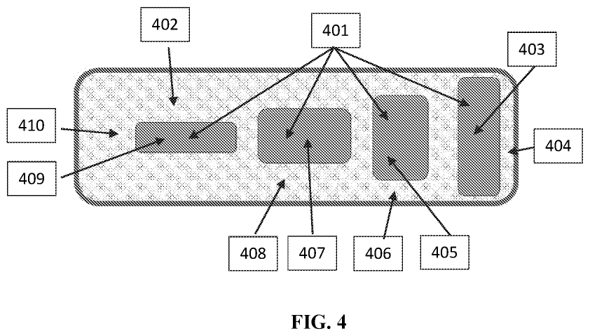

FIG. 4 depicts one example of a modified-release component comprising a matrix of differently-dissolving segments, as seen in a cross-section of a pill or table, having multiple reservoirs of bucillamine (401) surrounded by different matrix thicknesses (402). In this example, a first reservoir of bucillamine (403) is released in the stomach upon dissolution of a relatively thin matrix segment (404) that surrounds the first reservoir; a second reservoir (405) is released about four, about five, or about six hours later upon dissolution of a thicker segment (406) that surrounds the second reservoir; a third reservoir (407) is released about a further about four, about five, or about six hours later upon dissolution of an even thicker segment (408) that surrounds the third reservoir; and finally a fourth reservoir (409) is released another about four, about five, or about six hours later upon dissolution of the thickest segment (410) surrounding the fourth reservoir. One of skill in the art will readily envision variations of this arrangement in view of the present teachings. For example, in some embodiments, the intervals between periodic release may be more or less than about four, about five, or about six hours, varying within a critical range, as taught herein. In some embodiments, additional reservoirs and segments may be used to achieve additional pulses, e.g., a fifth, sixth, seventh, and/or eight pulse, released at critically-spaced intervals in accordance with aspects of the invention.

In some embodiments, a capsule filled with beads or microbeads is used, for example, a capsule that contains beads with a combination of different coatings designed to dissolve at step-wise, critically-spaced times, to release repeated pulses of bucillamine in accordance with aspects of the invention, e.g., in the same or different areas of the gastrointestinal tract. For example, in some embodiments, the beads are coated with the same or similar type of coating, but with different thicknesses that disintegrate to release drug at critically-spaced intervals of time. In some embodiments, the beads are coated with the different types of coating, but with the same or similar thicknesses that disintegrate to release drug at critically-spaced intervals of time. In some embodiments, the capsule may include immediate release beads or microbeads, providing a first pulse in the stomach upon ingestion.

Coatings may be selected for dissolution in particular environments of the gastrointestinal tract. For example, a “duodenum-dissolving coating” may comprise pH-sensitive materials, which remain intact at the lower pH environment of the stomach, but which disintegrate or dissolve at the pH commonly found in the small intestine of the patient. A coating may be selected of suitable thickness or composition, to resist stomach acids for the length of time in the stomach. Typically, a substantial amount or all of a particular coating is dissolved before the therapeutic agent is released from the bead or compartment enveloped by that coating, thereby achieving pulsed release of the therapeutic agent.

Different pH-sensitive materials may be used in the different coatings, where the different materials disintegrate or dissolve over a range of increasing pH, preferably in a step-wise manner, corresponding to the increasing pH along the small intestines. The pH of the small intestine gradually increases from about 4.5 to about 6.5, in the duodenal bulb, to about 7.2 in the distal portions of the small intestine (ileum). In some embodiments, a “duodenum-dissolving coating” may be designed to disintegrate or dissolve at a pH between about 5 to about 5.5; a “jejunum-dissolving coating,” at a pH between about 6 to about 6.5, and an “ileum-dissolving coating,” at a pH between about 7 to about 7.2. The composition thus may disintegrate or dissolve while transiting the small intestine, in periodic pulses in accordance with the invention. In preferred embodiments, multiple bead coatings dissolve in the stomach, at critically-spaced times.

Nonlimiting examples of materials, and combinations of materials, suitable for use in some embodiments of the present compositions, include beeswax and glyceryl monostearate; beeswax, shellac, and cellulose; cetyl alcohol, mastic, and shellac; shellac and stearic acid; polyvinyl acetate and ethyl cellulose; neutral copolymers of polymethacrylic acid esters; copolymers of methacrylic acid and methacrylic acid methylester, neutral copolymers of polymethacrylic acid esters containing metallic stearates; cellulose acid phthalates, and the like. Additional polymers for use in modified-release coatings are found, e.g., in Goodhart et al., Pharm. Tech., pp. 64-71, April 1984; and U.S. Pat. Nos. 2,809,918, 3,835,221, 4,432,966, 4,728,512, 4,794,001, and 5,225,202. Additional pH-sensitive materials for use in some embodiments of the present invention include those described in U.S. 2010/0310541 to Kessler et al, entitled “Compositions and Methods for Reducing the Toxicity of Certain Toxins, e.g., ¶¶[0058] and [0063]-[0065].

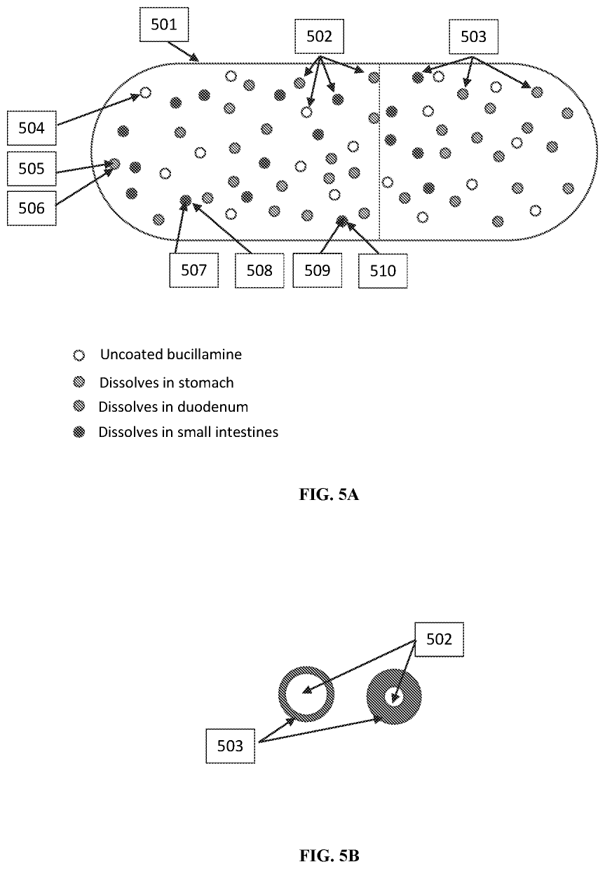

FIGS. 5A-5B depict one example of a modified-release component comprising a capsule of differently-coated beads, as seen in a cross-section of the capsule (501), with multiple bucillamine-containing beads (502) having different coatings (503), where the different coatings dissolve at step-wise, critically-spaced times to release repeated pulses of bucillamine in accordance with aspects of the invention, e.g., in the same or different areas of the gastrointestinal tract. In the example shown in FIG. 5A, a first set of beads (504) is uncoated to release bucillamine in the stomach; a second set of beads (505) also releases drug in the stomach, at a later time, upon dissolution of a stomach-dissolving coating (506), e.g., where the coating dissolves in low pH gastric juices; a third set of beads (507) release drug in the duodenum upon dissolution of a duodenum-dissolving coating (508), e.g., where the coating erodes under conditions specific to the duodenum, and a fourth set of beads (509) releases drug in the jejunum and/or ileum upon dissolution of a jejunum- and/or ileum-dissolving coating (510). FIG. 5B shows an alternative arrangement, where different sets of beads have coatings of different thicknesses, which dissolve at different times in the gastrointestinal tract. One of skill in the art will readily envision other variations of these arrangements in view of the present teachings. For example, in some embodiments, additional sets of beads may be used to achieve additional pulses, e.g., a fifth, sixth, seventh, and/or eight pulse, released at critically-spaced intervals in accordance with aspects of the invention. In some embodiments, a “small-intestine-dissolving coating” may be used instead of, or with, the duodenum-, jejunum-, and/or ileum-dissolving coatings, where the small-intestine-dissolving coating dissolves or disintegrates to release repeated, critically-spaced pulses of drug over time in the small intestines, preferably independent of its position along the small intestines.

The different coatings, as described herein, may serve to effect “gastrointestinal targeting,” that is, targeted release of contents that sequentially come into contact with particular regions of the gastrointestinal tract based on, e.g., respective prevailing pH ranges along the tract. In some embodiments, an anionic polymer or copolymer is used as the coating. Suitable anionic polymers or copolymers include, but are not limited to, cellulose glycolate (Duodcell®), cellulose acetate phthalate (CAP, cellulose acetate, cellulose acetate-phthalate), cellulose acetate succinate (CAS), cellulose acetate trimeliate (CAT), hydroxypropylmethylcellulose (HPMC), e.g., Methocell® E4M or Methocell® K100 available from Dow Chemical Co. of Midland, Mich.), hydroxypropylmethylcellulose phthalate (HPMCP, HP50, HP55), hydroxypropylmethyl cellulose acetate succinate (HPMCAS-LF, -MF, -HF), polyvinylacetate pthalate (PVAP, Sureteric®), vinyl acetate vinylpyrolidone-copolymer (PVAc, Kollidon® VA64), and Shell or varnish. The polymers or copolymers referred to can be formulated such that a pH-specific resolution is achieved. Polymers or copolymers also can be formulated such that gut flora-specific resolution is achieved, e.g., where coating dissolution occurs upon metabolism of different coatings by different gut flora, preferably in specific locations in the gastrointestinal tract, more preferably in the stomach, in repeated, critically-spaced pulses, as taught herein.

In some embodiments, a capsule with variable dissolution rates in different regions of the capsule is used. For example, the capsule may comprise “rings” or “bands” that dissolve at step-wise, critically-spaced times, to release repeated pulses of bucillamine in accordance with aspects of the invention, e.g., in the same or different areas of the gastrointestinal tract. The dissolving segments are often separated by intervening rings that do not dissolve (or do not substantially dissolve) but serve to support the capsule as other parts disintegrate (referred to herein as “non-dissolving rings”). In preferred embodiments, multiple bands dissolve in the stomach, at critically-spaced times.

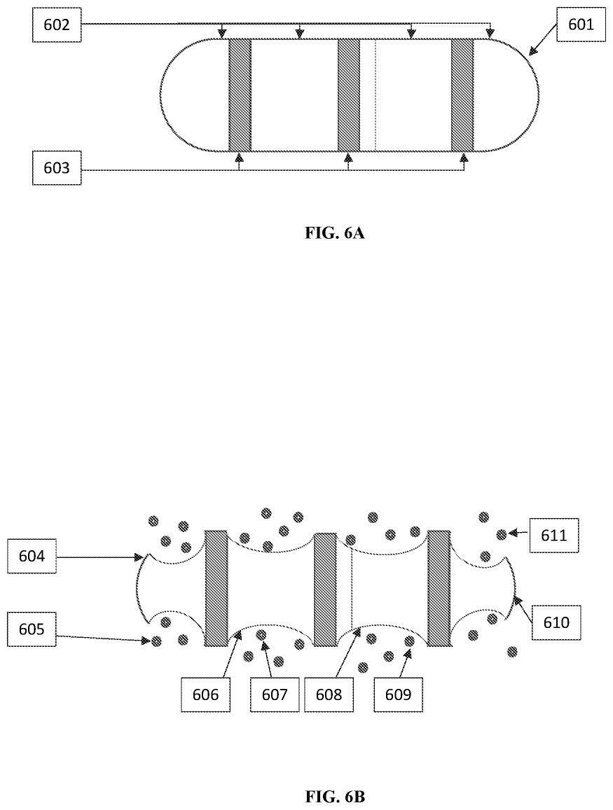

FIGS. 6A-6B depict one example of a modified-release component comprising a capsule of differently-dissolving bands, as seen in a cross-section of the capsule (601), separated into multiple bands (602) by intervening non-dissolving rings (603), where the different bands dissolve at step-wise, critically-spaced times to release repeated pulses of bucillamine in accordance with aspects of the invention, e.g., in the same or different areas of the gastrointestinal tract. In this example, as seen in FIG. 6B, a first band of capsule (604) dissolves to release a first pulse of bucillamine (605); a second band (606) dissolves to release a second pulse of bucillamine (607) about four, about five, or about six hours later; a third band (608) dissolves to release a third pulse of bucillamine (609) a further about four, about five, or about six hours later; and a fourth band (610) dissolves to release a fourth pulse of bucillamine (611) about another four, about five, or about six hours later. One of skill in the art will readily envision variations of this arrangement in view of the present teachings. For example, in some embodiments, the intervals between periodic release may be more or less than about four, about five, or about six hours, varying within a critical range, as taught herein. Further, in some embodiments, additional bands (with intervening rings) may be used to achieve additional pulses, e.g., a fifth, sixth, seventh, and/or eight pulse, released at critically-spaced intervals in accordance with the invention.

In some embodiments, the modified-release component comprises delivery systems using a more active expulsion of drug, under selected physiological conditions, which can include pH, salinity, or simply expulsion over time. Non-limiting examples include plugs and osmotic-push compartments. For example, a plug may be selected/designed that becomes unplugged specifically in the stomach (“a stomach-releasing plug”) to release contents of the plugged compartment (or most or substantially all of the contents of the plugged compartment) while in the stomach. An osmotic-push compartment can be selected/designed that first expands in the stomach due to osmolality differences between that compartment and the stomach, to bring about release of contents of that compartment (or most or substantially all of the contents of that compartment) in the stomach. Plugs and push compartments that release contents in other location of the gastrointestinal tract can be similarly selected and/or designed. A device utilizing osmotic push compartments generally will comprise an osmotic agent that imbibes water from the surrounding environment via a semi-permeable membrane. The osmotic agent may be an aqueous-swellable hydrophilic polymer, as osmogen, or osmagent. Additional osmotic devices for use in some embodiments of the present invention include those described in U.S. 2010/0310541 to Kessler et al, entitled “Compositions and Methods for Reducing the Toxicity of Certain Toxins, e.g., ¶¶ [0059], [0061], and [0077]-[0095]. In preferred embodiments, multiple push compartments release drug in the stomach, at critically-spaced times.

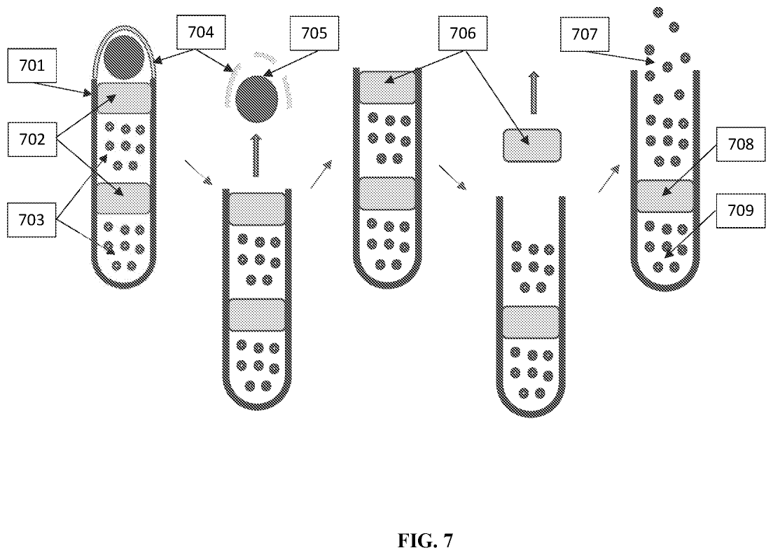

FIG. 7 depicts one example of a modified-release component comprising a capsule with differently-releasing plugs, as seen in a cross-section of the capsule (701), where the different plugs (702) separate multiple compartments of bucillamine (703) and become unplugged at step-wise, critically-spaced times to release repeated pulses of bucillamine in accordance with aspects of the invention, e.g., in the same or different areas of the gastrointestinal tract. In this example, a cap (704) at one end of the capsule dissolves to release a first pulse (bolus) of bucillamine (705); unplugging of the first plug (706) then releases a second pulse of bucillamine (707) about four, about five, or about six hours later; and unplugging of the second plug (708) releases a third pulse of bucillamine (709) another about four, about five, or about six hours later. One of skill in the art will readily envision variations of this arrangement in view of the present teachings. For example, in some embodiments, the intervals between periodic release may be more or less than about four, about five, or about six hours, varying within a critical range, as taught herein. Further, several of these systems can be stacked in the same capsule leading to multiple releases over time. For example, in some embodiments, additional plugs are used to achieve, e.g., a fourth, fifth, sixth, seventh, and/or eight pulse, released at critically-spaced intervals in accordance with aspects of the invention.

Another approach uses a capsule with individual packets, where a packet comprises drug and an osmotic-push compartment and the drug is “pushed” out of different packets at different times, e.g., based on changing osmolality at different locations along the gastrointestinal tract. For example, one push compartment may be first activated by absorption of fluid found in the stomach and this fluid causes the push compartment to expand and eject drug from the packet. Delivery systems using this approach are commercially available, e.g., OROS™ (Osmotic controlled Release Oral delivery System) from Alza.

Moreover, one of skill in the art will appreciate that one or more approaches for modified release, described herein, known in the art, and/or to be developed especially in view of the present disclosures, can be combined to bring about critically-spaced release of bucillamine pulses, in accordance with aspects of the invention.

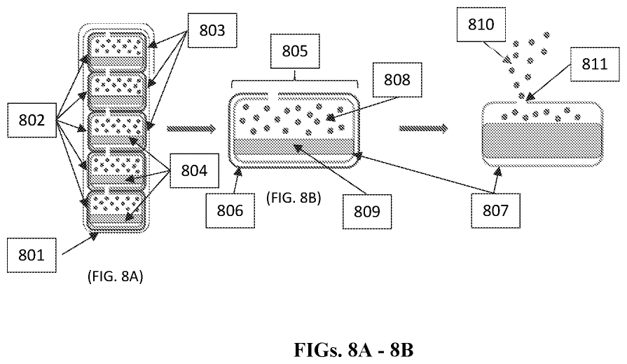

FIGS. 8A-8B depict one example of modified-release component combining approaches and comprising a capsule of packets with differently-dissolving coatings as well as differently-expanding osmotic-push compartments, as seen in a cross-section of the capsule (801) and packets (802). The different coatings (803) dissolve at step-wise, critically-spaced times, e.g., in the same or different areas of the gastrointestinal tract; and the different osmotic-push compartments (804) expand at step-wise, critically-spaced times, e.g., in the same or different areas of the gastrointestinal tract, working together to release repeated pulses of bucillamine over time in accordance with aspects of the invention. FIG. 8B is a blown-up view of one packet.

In the example shown in FIG. 8B, the capsule is composed of a gelatin that dissolves in the stomach, releasing five distinct packets. Individual packets have an external membrane or coating that dissolves at different times and/or in the appropriate part of the gastrointestinal tract. For example, the first packet (805) is enclosed by a first coating (806) that first dissolves in the stomach; the first packet also contains a first semi-permeable or osmotic membrane (807) surrounding a first bucillamine compartment (808) and a stomach-expanding osmotic-push compartment (809). The push compartment (809) first expands in the stomach, due to osmolality differences between the stomach-expanding compartment and the stomach, to release a first pulse (810) of bucillamine that is pushed through a first small orifice or aperture (811) in the first semi-permeable membrane.

The same or similar process occurs for remaining packets. For example, in this example, a second packet is enclosed by a second coating that first dissolves in the duodenum, the second packet also containing a second semi-permeable membrane surrounding a second bucillamine compartment and a duodenum-expanding osmotic-push compartment. This push compartment first expands in the duodenum, due to osmolality differences between the duodenum-expanding compartment and the duodenum, to release a second pulse of bucillamine that is pushed through a second small orifice or aperture found on the second semi-permeable membrane. Further, for example in this embodiment, a third packet is enclosed by a third coating that first dissolves in the jejunum, the third packet also containing a third semi-permeable membrane surrounding a third bucillamine compartment and a jejunum-expanding osmotic-push compartment, which first expands in the jejunum due to osmolality differences between the jejunum-expanding compartment and the jejunum, to release a third pulse of bucillamine that is pushed through a third small orifice or aperture found on the third semi-permeable membrane, and so on for fourth and fifth packets, having ileum-expanding and colon-expanding osmotic-push compartments, that expand to release fourth and fifth pulses of bucillamine in the ileum and colon, respectively. One of skill in the art will readily envision variations of this arrangement in view of the present teachings. For example, in some embodiments, additional packets are used to achieve additional pulses, released at critically-spaced intervals in accordance with the invention. In some embodiments, a “small-intestine-expanding” osmotic-push compartment may be used instead of, or with, a duodenum-, jejunum-, and/or ileum-expanding push compartments, where the small-intestine-expanding compartment expands to bring about release of drug in repeated, critically-spaced pulses over time in the small intestines, preferably independent of its position along the small intestines.

In some embodiments, the modified-release component comprises a matrix comprising differently-releasing polymers, where the different polymers release bucillamine at step-wise, critically-spaced times in accordance with the invention, e.g., in the same or different areas of the gastrointestinal tract. Matrices may be biodegradable or mineral-based and may comprise macro- or micro-porous systems. Varying geometry and permeability of a matrix allows for various types of drug release profiles. Generally speaking, the rate at which a drug is released from a matrix is proportional to the surface area exposed over time. Changing the surface area or changing the time of exposure can alter the amount of drug released and the timing thereof. For example, in some embodiments, a tablet is embedded with multiple reservoirs, containing the same or various amounts of drug, and coated with a polymer matrix comprising differently-releasing polymers through which the embedded drug leeches out over time, in accordance with the present disclosures. In preferred embodiments, multiple pulses of bucillamine are released in the stomach from the polymers.

In some embodiments, the modified-release component comprises a matrix of a “stomach-releasing polymer” that releases its cargo (or most or substantially all of its cargo) in the stomach; a “duodenum-releasing polymer” that releases its cargo (or most or substantially all of its cargo) in the duodenum; a “jejunum-releasing polymer” that releases its cargo (or most or substantially all of its cargo) in the jejunum; an “ileum-releasing polymer” that releases its cargo (or most or substantially all of its cargo) in the ileum; and/or “colon-releasing polymer” that releases its cargo (or most or substantially all of its cargo) in the colon. One of skill in the art can select matrix polymers to effect release of bucillamine (as the cargo) in specific regions of the gastrointestinal tract (see, e.g., Patel et al., 2011, “Matrix Type Drug Delivery System: A Review” Journal of Pharmaceutical Science and Bioscientific Research, 1(3): 143-151). In some embodiments, a “small-intestine-releasing polymer” may be used instead of, or with, the duodenum-, jejunum-, and/or ileum-releasing polymers, where the small-intestine-releasing polymer releases repeated, critically-spaced pulses of drug over time in the small intestines, preferably independent of its position along the small intestines.

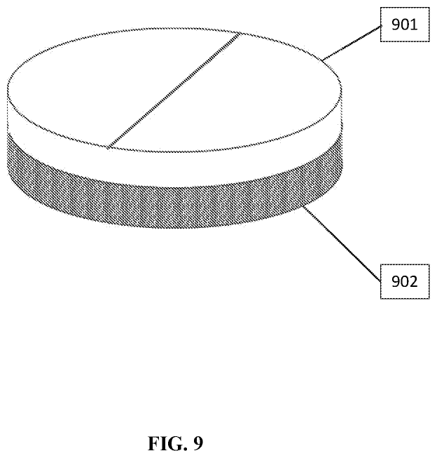

In some embodiments, the modified-release component comprises a matrix of layers of drug and drug embedded polymers to achieve an immediate release in the stomach, followed by periodic pulses of drug release. FIG. 9 depicts one example of a simple modified-release approaching using a matrix comprising one layer of a stomach-releasing polymer (902) and one layer for immediate-release of drug (901). In this simple example, drug contained in the immediate-release layer (901) is released into the stomach upon ingestion. The second layer (902) comprises bucillamine embedded in a polymer that dissolves over time in gastric fluids, to deliver a pulse of bucillamine in the stomach, e.g., about four to about eight hours after the immediate release, or about two to about six hours after the immediate release. Timing of the releases generally depends on concentration and type of polymer used. VersaTab® system, e.g., can be used to design a pill/tablet with two such layers. One of skill in the art will readily envision variations of this arrangement in view of the present teachings. For example, in some embodiments, the intervals between periodic release may be more or less than about four, about five, or about six hours, varying within a critical range, as taught herein. In some embodiments, additional layers of polymers may be used to achieve additional pulses, e.g., a third, fourth, fifth, sixth, seventh, and/or eight pulse, released at critically-spaced intervals in accordance with the invention.

The matrix polymers used may be hydrophilic polymers, hydrophobic polymers, lipids, plastics, or any combination thereof. Generally, drug is mixed with one or more polymer types, a variety of which are commercially available. Examples of hydrophilic polymers for use in the modified-release component include, but are not limited to, Hydroxypropylmethylcellulose (HPMC), Hydroxypropylcellulose (HPC), Hydroxyethyl cellulose (HEC), Xanthan gum, Sodium alginate, Poly (ethylene oxide) and cross-linked homopolymers and copolymers of Acrylic acid. Non-limiting examples of hydrophobic polymers include polyethylene, polyvinyl chloride, ethyl cellulose and acrylate polymers and their copolymers. Non-limiting examples of lipids include carnauba wax with stearyl alcohol or stearic acid. Other polymers for use in certain embodiments are those described in, e.g., U.S. 2008/0255073 to Gallop et al., entitled “Compounds for sustained release of orally-delivered drugs;” U.S. 2003/0147844 to Choe et al., entitled “Polymeric thiol-linked prodrugs;” U.S. 2003/0157052 to Choe et al., entitled “Polymeric thiol-linked prodrugs employing benzyl elimination systems;” U.S. 2010/0069293 to Bolotin et al., entitled “Polymeric carrier compositions for delivery of active agents methods of making and using the same;” U.S. 2016/0279056 to Zhao et al., entitled “Liquisoft capsules;” WO 2013/004999 to Biocopea Ltd, entitled “Drug combinations and uses in treating a coughing condition.” Additional polymeric matrixes for use in some embodiments of the present invention include those described in U.S. 2010/0310541 to Kessler et al, entitled “Compositions and Methods for Reducing the Toxicity of Certain Toxins, e.g., ¶¶ [0060], [0062] (describing terpolymers), and [0069]-[0074] (describing erodable and non-erodable matrices). See also, e.g., expired U.S. Pat. No. 6,613,807 to Uhrich, entitled “Therapeutic polyanhydride compounds for drug delivery” (describing polymers that release a biologically active compound upon hydrolysis of the polymer); WO 2000/000179, entitled “Solid dispersed preparation of poorly water-soluble drug containing oil, fatty acid or mixtures thereof” (disclosing solid dispersed preparations for releasing drugs in the gastrointestinal tract); and WO 2014/111957, entitled “Nitric oxide releasing prodrugs of therapeutic agents” (describing nitric oxide releasing prodrugs). Commercially available releasing polymers also may be used, e.g., VersaTab® technology may be employed to deliver staggered release of drugs.

Other approaches for controlling release of drug that may or may not be used with the compositions or delivery systems disclosed herein include, e.g., polymer coatings according to U.S. 2007/0249735 to Chopdekar et al., entitled “Halide-free glucosamine-acidic drug complexes;” ionic liquids of U.S. 20070093462 to Rogers et al., entitled “Multi-functional ionic liquid compositions for overcoming polymorphism and imparting improved properties for active pharmaceutical, biological, nutritional, and energetic ingredients;” or additional approaches described in U.S. 2010/0310541 to Kessler et al., entitled “Compositions and Methods for Reducing the Toxicity of Certain Toxins;” U.S. 2015/0328323 to Satyam, entitled “Nitric Oxide Releasing Pro-drugs of Therapeutic Agents;” and U.S. 2017/0129867 to Nguyen et al., entitled “Tiopronin prodrugs, pharmaceutical compositions thereof, and methods of use.”

In some embodiments, the modified-release component comprises a matrix comprising differently-adhering polymers, where the different polymers adhere to, and release bucillamine at step-wise, critically-spaced times in accordance with aspects of the invention, e.g., in the same or different areas of the gastrointestinal tract. The polymers may remain attached for periods of time to mucosal or epithelial membranes, thereby extending dwell time, or residence time, in particular locations in the gastrointestinal tract, such as in the stomach, while bucillamine is released in critically-spaced pulses, as taught herein. In some embodiments, the a bioadhesive polymer increases residence time of an orally administered therapeutic agent (bucillamine), compared to its residence time when orally administered in a composition lacking the bioadhesive polymer, by at least about 10%, at least about 20%, at least about 30%, at least about 40%, at least about 50%, at least about 60%, at least about 70%, or at least about 80%, and/or up to about 40%, up to about 50%, up to about 60%, up to about 70%, up to about 80%, or up to about 90%, in a specific region, such as in the stomach or in the small intestine.

In some embodiments, an oral multiparticulate form may be used comprising pellets containing bucillamine, embedded in a matrix of polymers with different bioadhesive effects, such as a “stomach-adhering polymer” that adheres to the stomach and releases its cargo (or most or substantially all of its cargo) in the stomach; a “duodenum-adhering polymer” that adheres to the duodenum and releases its cargo (or most or substantially all of its cargo) in the duodenum; a “jejunum-adhering polymer” that adheres to the jejunum and releases its cargo (or most or substantially all of its cargo) in the jejunum; an “ileum-adhering polymer” that adheres to the ileum and releases its cargo (or most or substantially all of its cargo) in the ileum; and/or “colon-adhering polymer” that adheres to and releases its cargo (or most or substantially all of its cargo) in the colon. One of skill in the art can select matrix polymers to adhere to (and effect release of bucillamine as the cargo) in specific regions of the tract. In some embodiments, a “small-intestine-adhering polymer” may be used instead of, or with, the duodenum-, jejunum-, and/or ileum-adhering polymers, where the small-intestine-adhering polymer releases repeated, critically-spaced pulses of drug over time in the small intestines, preferably independent of its position along the small intestines. For example, one of the bioadhesive polymers may be selected to bind at regions+/−0.5 pH units, preferably +/−0.3 pH units, relative to the pH at which an outer enteric coating starts to dissolve. The bioadhesive polymers may be selected to have desired bioadhesive effects, as described herein, based on parameters known in the art, e.g., having a water absorption of 10 to 750%, preferably 10 to 250%, more preferably 10 to 160% by weight in about 15 minutes (see, also e.g., WO 2005/007139 to Lizio et al., entitled “Multiparticle Pharmaceutical Dosage Form Containing A Mucoadhesively Formulated Peptide Or Protein Active Substances Method For Producing Said Pharmaceutical Dosage Form”). Suitable differently-adhering polymers include, but are not limited to, chitosan and derivatives thereof, (meth) acrylate copolymers, celluloses, in particular methyl celluloses such as sodium carboxymethylcellulose (e.g., Blanose, Methocel®), and the like, or combinations thereof. In preferred embodiments, the bioadhesive polymers comprise (meth) acrylate copolymers. Additional bioadhesive materials for use in some embodiments of the present invention include those described in U.S. 2010/0310541 to Kessler et al, entitled “Compositions and Methods for Reducing the Toxicity of Certain Toxins, e.g., ¶ [0066]. In some embodiments, adherence factors called adhesins may be used as the bioadhesive polymer. Adhesins that may be used include those derived from fimbriae of bacteria, or synthetic analogs thereof, that adhere to cell surfaces to slow the movement of drug within a specific region of the gastrointestinal tract.

In a particular embodiment, the matrix of differently-adhering polymers itself is enterically coated, e.g., with an outer coating comprising an anionic polymer or copolymer, that releases pellets in the matrix beyond the stomach (e.g., based on the pH range of the stomach, thickness of the coating, choice of anionic polymer and/or additional excipients, etc.). For example, the outer coating may dissolve in area of pH 4.0 to 8.0 in the intestine, and/or within 15 to 60 min of being swallowed, releasing the matrix of bioadhesive polymers that differentially bind along different regions of the intestinal mucosa, and there release the therapeutic (bucillamine). The outer coating preferably has little or no interactions with therapeutic (bucillamine) nor with the bioadhesive polymer matrix. In some embodiments, multiple coatings are used, containing the same or different bioadhesive polymer matrix, where the different coatings differentially dissolve at step-wise, critically-spaced times in accordance with aspects the invention, in the same or different areas of the gastrointestinal tract.

In some embodiments, the modified-release component comprises a timed device, such as an electronic device, a microfluidic device, a microprocessor, a nanotechnology device, and/or other device that is programmed/designed to facilitate timed release of a therapeutic agent. For example, microprocessors or other mechanical devices may be used to time delivery, in some cases providing precisely timed releases of drug. In a particular embodiment, the device used is a Intellicap Electronic Capsule, commercially available from Medimetrics (Eindhoven, The Netherlands), which comprises a microfluidic pump controlled by an integrated microprocessor and powered by an on-board battery. One of skill in the art will appreciate that developing technology is creating additional approaches for timing drug delivery, any of which may be used in the alternative or in combination with one or more modified-release components described herein.

Gastro-Retentive Delivery

In preferred embodiments, oral formulations of the present invention achieve repeated, critically-spaced, pulsed delivery of bucillamine in the stomach. In a simple approach, the first release of drug may be achieved using an immediate-release component, from which drug is released in a first pulse following ingestion. In preferred embodiments, the first and second releases occur in the stomach, more preferably, first, second, and third releases occur in the stomach, and even more preferably, said first, second, third, and fourth releases occur in the stomach.

Delivery in the stomach may be achieved using a modified-release approach that includes a gastro-retentive feature. A gastro-retentive feature can be used to target drug delivery specifically in the stomach, preferably achieving multiple critically-spaced pulses of bucillamine in the stomach. A “gastro-retentive feature” generally refers to a gastro-retentive drug delivery system that prolongs residence time of an orally administered therapeutic agent (bucillamine) in the stomach, compared to the agent's stomach residence time when orally administered in a composition lacking the gastro-retentive feature. In some embodiments, stomach residence time is increased by at least about 10%, at least about 20%, at least about 30%, at least about 40%, at least about 50%, at least about 60%, at least about 70%, or at least about 80%. In some embodiments, stomach residence time is increased up to about 40%, up to about 50%, up to about 60%, up to about 70%, up to about 80%, or up to about 90%.

In some embodiments, the gastro-retentive feature is at least one selected from the group consisting of a stomach-adhering coating, a floatation system, a sedimentation system, and an expandable system. In some embodiments, as noted above, the modified-release component is used in conjunction with an immediate-release component, allowing the first release of drug to occur in the stomach from the immediate-release component.

In some embodiments, the gastro-retentive feature comprises a stomach-adhering coating, encapsulating, or partially encapsulating, the therapeutic agent. The stomach-adhering coating may comprise any one or more of the bioadhesive stomach-adhering polymers described above. In particular embodiments, the stomach-adhering coating becomes attached to the lining of the stomach, increasing stomach residence time, to bring about increased release (and uptake) of the drug in the stomach, compared to the non-coated therapeutic. Increased stomach release and uptake of bucillamine surprisingly increases bioavailability, increasing free bucillamine plasma levels, as well as total drug recovery in the urine.

A stomach-adhering coating may coat, or partially coat, the therapeutic agent itself, or may coat, or partially coat, other structures of a modified-release component, as described herein or known in the art, to provide a partial coating. For example, in some embodiments, the stomach-adhering coating comprises stomach-adhering polymers that coat or partially coat at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor. A “partial coating” generally refers to a coating that envelops, encompasses, or covers less than 100% of the entire outer surface of a composition, formulation, component, or system, such as enveloping less than 100% of a modified-release component described herein for bringing about repeated, critically-spaced pulses over a period of time. In some embodiments, the partial coating covers about 20%, about 30%, about 40%, about 50%, about 60%, about 70%, about 80%, about 90% of the composition, formulation, component, or system being partially coated. Partial coating allows release of drug from the un-coated regions of the composition, formulation, component, or system, e.g., release in critically-spaced pulses, as described herein. A “full coating,” “complete coating,” or similar term, generally refers to a coating that envelops, encompasses, or covers about 100%, or substantially all, of the entire outer surface of a composition, formulation, component, or system, such as enveloping about 95%, about 98%, about 99%, or about 100% of a modified-release component described herein for bringing about repeated, critically-spaced pulses over a period of time. A full coating may allow release of drug from the regions of the composition, formulation, component, or system, e.g., release in critically-spaced pulses, as described herein, by dissolving or disintegrating following to exposure to the stomach environment, while, in preferred embodiments, the region of the coating anchored to the stomach lining remains intact, or substantially intact, for a longer period, e.g., for a time suitable to increase stomach residence time, as described herein, in that the anchored region of the coating is protected from the stomach environment to a greater extent than the un-anchored region of the coating.

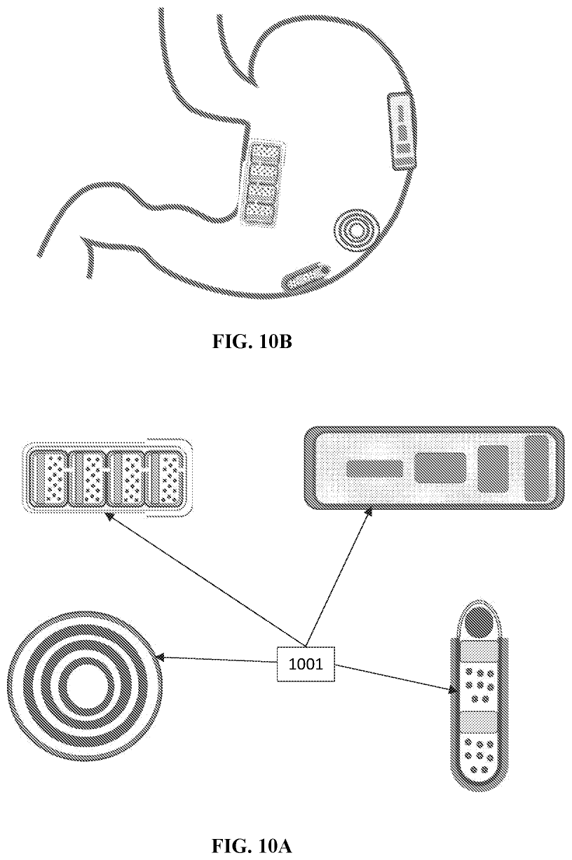

FIGS. 10A-10B. depict several examples of modified-release components, described above, that further are each coated by a stomach-adhering coating as the gastro-retentive feature, in accordance with aspects of the present invention. FIG. 10A depicts a coating of stomach-adhering polymers (1001), coating each of a “jaw-breaker” of layered coatings, a matrix of differently-dissolving segments, a capsule comprising differently-releasing plugs, and a capsule of packets with differently-dissolving coatings that themselves envelop differently-expanding osmotic-push compartments.

FIG. 10B depicts adherence of the stomach-adhering coating in each case to the stomach lining, “anchoring” the composition in the stomach, and thereby effecting release of one or more pulses of drug in the stomach. In this example, the composition becomes attached to the stomach lining via its coated region, while the uncoated region, or region where the composition remains exposed to the stomach environment, which may play a role in effecting drug release, as described herein. One of skill in the art will readily envision variations of this arrangement in view of the present teachings. For example, in some embodiments, the stomach-adhering coating is used to coat one or more other modified-release components described herein; and/or is used in combination with an immediate-release component, such as where the stomach-adhering coating itself is enveloped by an outermost layer of bucillamine that is immediately released upon ingestion.

In some embodiments, the gastro-retentive feature comprises a sedimentation system. A sedimentation system uses a high density agent that can be combined with the therapeutic agent to provide a formulation possessing density greater than that of stomach contents and/or gastric fluids, that is, greater than about 1.004 gm/cm³. Greater density causes the formulation or dosage form to sink to the bottom region of the stomach, preferably being caught, like sediment, in the folds of the stomach “floor,” or rugae of the stomach near the pyloric region, where stomach contents generally can withstand peristaltic movements that normally would empty other parts of the stomach. This sinking and/or sedimentation slows exit of the formulation from the stomach, increasing stomach residence time and allowing multiple releases (and uptake) of bucillamine pulses in the stomach.

High density agents include any agents that may be combined with a therapeutic agent to create a high density formulation. A “high density agent” or “high density formulation” is an agent or formulation possessing density greater than that of stomach contents and/or gastric fluids of a typical, healthy adult, that is, a density exceeding about 1.002 gm/cm³, about 1.003 gm/cm³, about 1.004 gm/cm³, about 1.005 gm/cm³, about 1.006 gm/cm³, about 1.007 gm/cm³, or about 1.008 gm/cm³; and/or up to about 1.006 gm/cm³, about 1.007 gm/cm³, or about 1.008 gm/cm³, about 1.009 gm/cm³, or about 1.01 gm/cm³. In some embodiments, the high density agent is a dense excipient, such as but not limited to, titanium oxide, zinc oxide, iron powder, or barium sulphate. In a preferred embodiment, bucillamine is embedded in pellets that have a density greater than 1.004 gm/cm³.

A sedimentation system may comprise a high density agent combined with the therapeutic agent itself, or with other structures of a modified-release component, as described herein, known in the art, or to be developed, especially in view of the present disclosures. For example, in some embodiments, the sedimentation system comprises a high density agent combined in a pellet with at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor. In a preferred embodiment, the high density pellet is used in combination with an immediate-release component, such as where the pellet itself is enveloped by an outermost layer of bucillamine that is immediately released upon ingestion; or where high density pellets are combined with immediate-release pellets to effect at least two critically-spaced pulses.

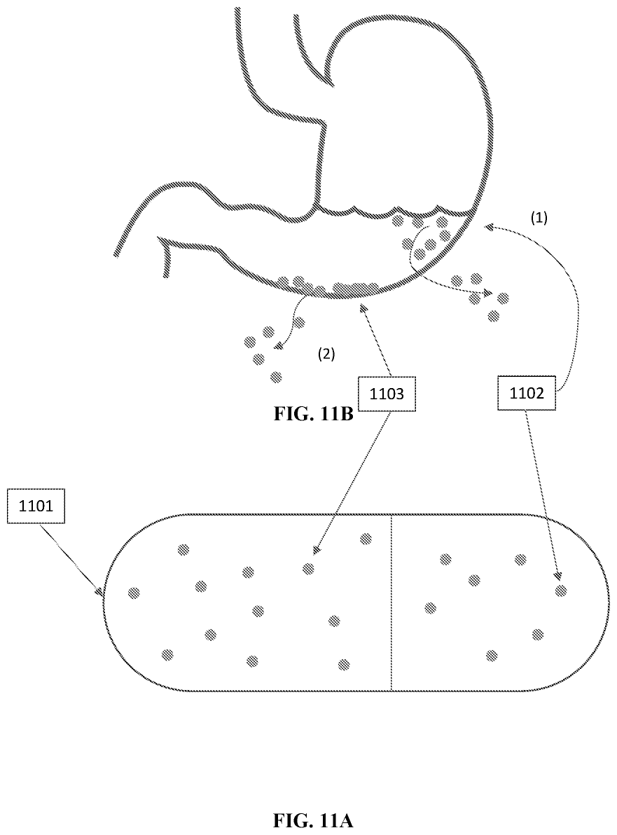

FIG. 11 depicts one example of a sedimentation system for modified-release, comprising immediate release pellets and high density pellets as the gastro-retentive feature. FIG. 11 depicts a capsule (1101), shown in cross section, comprising a combination of immediate release bucillamine pellets (1102) and high density pellets (1103) that comprise bucillamine and a high density agent. FIG. 11 also depicts immediate release of bucillamine at a first time point (1), in a first pulse, where the drug is quickly absorbed through the stomach wall, followed by a second release of bucillamine at a second time point (2), in a second pulse, about four, about five, or about six hours later. One of skill in the art will readily envision variations of this arrangement in view of the present teachings. For example, in some embodiments, one or more other modified-release components described herein may be combined with a high density excipient, to achieve, e.g., third, fourth, fifth, sixth, seventh, and/or eight pulses, in the stomach and/or small intestine, at critically-spaced intervals in accordance with aspects of the invention.

In some embodiments, the gastro-retentive feature comprises an expandable system. An expandable system generally comprises at least one structure or agent that expands, swells, enlarges, grows, opens, or otherwise gets bigger in the stomach, to attain a size that physically prevents, impedes, reduces, or slows exit of stomach contents and/or itself from the stomach, such as by blocking passage through the pyloric sphincter. In some embodiments, the expandable system comprises a swellable structure, such as a gel that swells upon contact with gastric fluids, or a shape-modifying structure. In some embodiments, the expandable system comprises a shape-modifying or swellable structure that impedes exit from the stomach of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor. In some embodiments, the expandable structure serves both a gastro-retentive function, as well as achieving modified-release in pulses.

In some embodiments, for example, the expandable system comprises a shape-modifying structure, such as a folded form contained in a capsule that unfolds to a larger size upon dissolution/disintegration of the capsule in the stomach environment. Folded forms may be one or more, e.g., of a 4-lobed structure, a disc, a 4-limbed cross, a ring, a tetrahedron, a star and the like. One of skill in the art will appreciate that one or more of these folded structures, used as an expandable gastro-retentive feature, can further be designed to act as a modified-release component, e.g., where different arms or regions of the structure release drug pulses at step-wise, critically-spaced times, in accordance with the aspects of invention, in the stomach.

For example, a folded star-shaped structure can be used that carries bucillamine in its different arms. The structure is loaded into a capsule, which dissolves in the stomach upon ingestion, allowing the device to unfurl. Since the expanded device cannot pass through the pylorus, it remains in the stomach, releasing drug located in each arm according to that arm's delivery method. In some embodiments, the different arms comprise bucillamine and differently-releasing polymers, where the different polymers release drug at step-wise, critically-spaced times. For example, arms may contain reservoirs of drug surrounded by matrices that vary in thickness and that dissolves the stomach (low pH environment) at different rates. When the entire star structure dissolves, it may pass safely through the remainder of the gastrointestinal tract. For structures with more arms than desired pulses, one or more arms may be composed of inert material, without bucillamine. See, e.g., gastro-retentive devices commercially available from Lundra, Inc. See also, e.g., https://www.biocentury.com/bc-innovations/product-rd/2016-12-07/how-lyndra%E2%80%99s-new-star-shaped-pill-could-deliver-drug-weeks-time; and https://gizmodo.com/freaky-expanding-pill-stays-in-your-gut-for-days-to-del-1789091092; and

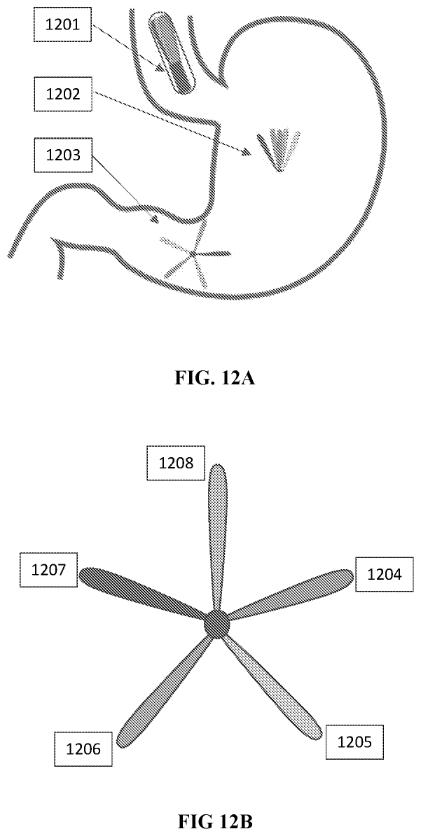

FIGS. 12A-B depict one example of a gastro-retentive expandable system comprising a shape-modifying structure. FIG. 12A depicts the structure folded for oral delivery into a capsule (1201), which rapidly dissolves in the stomach, allowing the structure to unfurl (1202) and, once unfurled, prevented from passing through the pylorus (1203). While exposed in the stomach, gastric juices interact with the arms of the structure. In this example, each arm comprises bucillamine and a matrix of polymers that release bucillamine in the stomach in repeated, critically-spaced pulses over a period of time. FIG. 12B depicts the different matrices in different colors, where the matrix of the first arm (1204) disintegrates to release a first pulse of drug; the matrix of the second arm (1205) disintegrates to release a second pulse of drug about four, about five, or about six hours later; the matrix of the third arm (1206) disintegrates to release a third pulse of drug another about four, about five, or about six hours later; the matrix of the fourth arm (1207) disintegrates to release a fourth pulse of drug a further about four, about five, or about six hours later; and the matrix of the fifth arm (1208) disintegrates to release a fifth pulse of drug a still further about four, about five, or about six hours later. One of skill in the art will readily envision variations of this arrangement in view of the present teachings. For example, in some embodiments, the intervals between periodic release may be more or less than about four, about five, or about six hours, varying within a critical range, as taught herein. In some embodiments, arms or other structures may be used to achieve additional pulses, e.g., a sixth, seventh, and/or eight pulse, released at critically-spaced intervals in accordance with aspects of the invention. Further, a different modified-release strategy may be employed, e.g., where the different regions or arms of the shape-modifying structure comprise modified-release components comprising different coatings, differently-coated beads, reservoirs surrounded by matrices of different thicknesses, differently-releasing plugs, differently-expanding osmotic-push compartments, differently-timed microprocessors, and the like, and any combinations thereof, e.g., that govern the timing of release of drug into the stomach.

In some embodiments, the expandable system comprises a swellable structure, such as a gel that swells upon contact with gastric fluids, to form a structure that physically prevents, impedes, reduces, or slows exit of stomach contents and/or itself from the stomach, such as by blocking passage through the pyloric sphincter. In some embodiments, the swollen structure serves both a gastro-retentive function, as well as achieving modified-release. For example, in some embodiments, in-situ gel formation creates a reservoir from which drug can be released in critically-spaced periodic pulses. Formation of the gel may arise from exposure to selected physiologic factors, e.g., gastric fluids in the stomach, having relatively low pH. Examples of materials for use in forming in-situ gels for gastro-retention include, but are not limited to, gellan gum, alginic acid, xyloglucan, pectin, chitosan, poly-caprolactone, poly-lactic acid, poly-lactic-co-glycolide, and the like, or other gel materials known in the art that form or swell in the stomach environment.

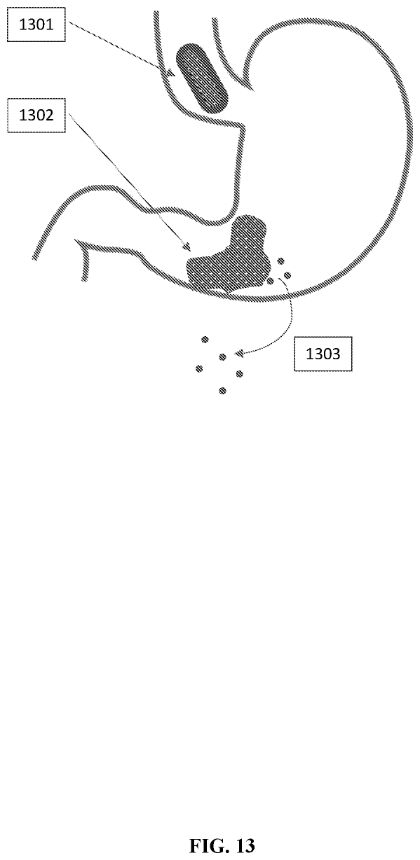

FIG. 13 depicts one example of a gastro-retentive expandable system comprising a swellable structure. In this example, the swellable structure is formed by in-situ gel formation. A pill, tablet, or capsule is swallowed (1301). The contents of the pill, tablet, or capsule combine with gastric fluids to form a swollen “gel,” that cannot pass through the pylorus in its enlarged state (1302). Drug eludes from the gel (1303), in critically-spaces pulses, and is absorbed in the stomach. One of skill in the art will readily envision variations of this arrangement in view of the present teachings. For example, the swellable structure may be used in conjunction with one or more other modified-release strategies, such as those described herein.

In some embodiments, the gastro-retentive feature comprises a floatation system. A floatation system uses an agent that can be combined with a therapeutic to provide a formulation possessing density lower than that of stomach contents and/or gastric fluids, that is, lower than about 1.004 gm/cm³. Lower density causes the formulation or dosage form to rise to the surface of the stomach contents, above the pylorus, avoiding normal gastric emptying. This floating thus slows exit from the stomach and increases stomach residence time, allowing multiple releases (and uptake) of bucillamine in the stomach. Floatation systems generally use effervescent or non-effervescent agents to lower bulk density.

Density-lowering agents include any agents that may be combined with a therapeutic agent to create a low density formulation. A “density-lowering agent can be any agent that increases buoyancy and/or lowers density of a drug-containing formulation or device, to a density below that of stomach contents and/or gastric fluids of a typical, healthy adult, that is, to a density less than about 1.006 gm/cm³, about 1.005 gm/cm³, about 1.004 gm/cm³, about 1.003 gm/cm³, about 1.002 gm/cm³, about 1.001 gm/cm³, or about 1.000 gm/cm³; and/or down to about 1.003 gm/cm³, about 1.002 gm/cm³, or about 1.001 gm/cm³, about 1.000 gm/cm³, or about 0.999 gm/cm³.” A “low density agent” or “low density formulation” is an agent or formulation possessing density lower than that of stomach contents and/or gastric fluids of a typical, healthy adult, that is, a density less than about 1.006 gm/cm³, about 1.005 gm/cm³, about 1.004 gm/cm³, about 1.003 gm/cm³, about 1.002 gm/cm³, about 1.001 gm/cm³, or about 1.000 gm/cm³; and/or down to about 1.003 gm/cm³, about 1.002 gm/cm³, or about 1.001 gm/cm³, about 1.000 gm/cm³, or about 0.999 gm/cm³.

A floatation system may comprise a density-lowering agent combined with the therapeutic agent itself, or with other structures of a modified-release component, as described herein, known in the art, or to be developed especially in view of the present disclosures. For example, in some embodiments, the floatation system comprises an effervescent and/or a non-effervescent agent that increases buoyancy of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; and a matrix comprising differently-adhering polymers. In a preferred embodiment, the density-lowering agent is used in combination with an immediate-release component, such as where the low density formulation is enveloped by an outermost layer of bucillamine that is immediately released upon ingestion, for example, to effect at least two critically-spaced pulses in the stomach.

In some embodiments, the gastro-retentive floatation system comprises a non-effervescent agent to lower density and increase buoyancy. Examples of density-lowering non-effervescent agents include, but are not limited to, colloid gels, microporous compartments, floating microspheres, alginate beads, raft-forming structures, and microballoons. For example, the density-lowering, non-effervescent agent may comprise swellable cellulose type hydrocolloids, polysaccharides, or matrix-forming polymers such as polycarbonate, polystyrene, polyacrylate, or polymethacrylate. Air trapped inside the density-lowering non-effervescent agent, e.g. a swollen polymer or buoyant polymer, generally makes it buoyant in the stomach fluids, thereby delaying movement to other parts of the gastrointestinal tract. The density-lowering non-effervescent agent may be embedded with bucillamine and one or more excipients, such as but not limited to sodium alginate, polyvinyl acetate, hydroxypropyl methylcellulose polyacrylates, calcium chloride, polyethylene oxide, carbopol, agar, and polycarbonates. In a particular embodiment, the gastro-retentive floatation system is combined with drug in a microsphere, such as microspheres comprising one or more polymers that may be selected from albumin, starch, gelatin, polyacrylamine, polymethacrylate, and polyalkylcyanoacrylate.

The gastro-retentive floatation system comprising a non-effervescent agent may be provided in multiple or single unit dosage forms, as illustrated below.

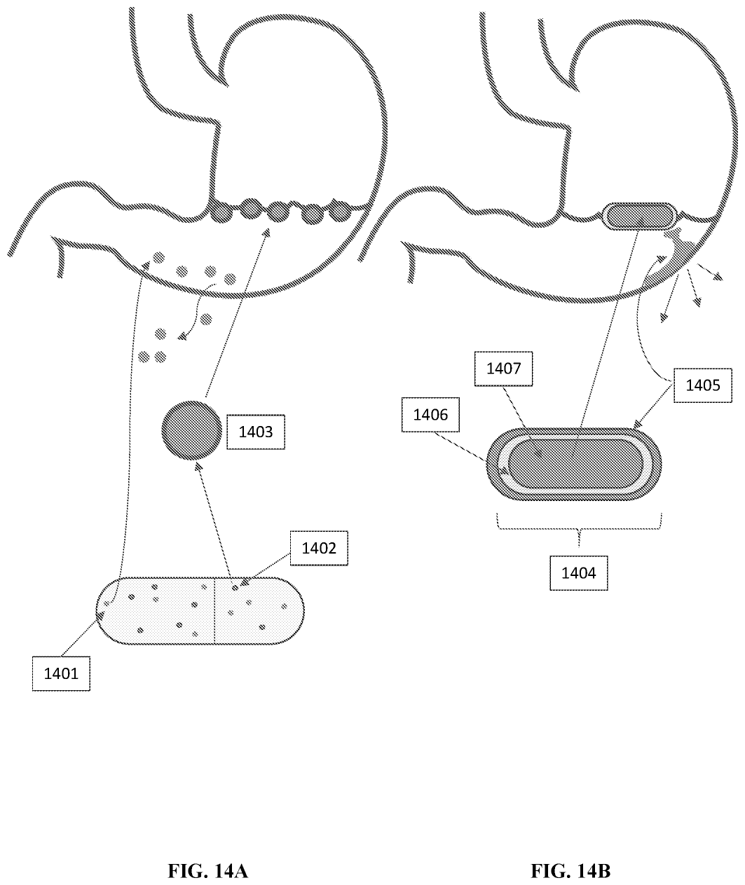

FIGS. 14A-14B depict examples of multiple unit (FIG. 14A) and single unit (FIG. 14B) dosage forms of gastro-retentive floatation systems for modified-release, comprising immediate release bucillamine microbeads and bucillamine microbeads coated in a non-effervescent density-lowering matrix of buoyant polymers, as the gastro-retentive feature. FIG. 14A depicts a multiple unit capsule (1401), shown in cross section, comprising the immediate release bucillamine microbeads (1402) and microbeads coated or encased in the buoyant polymer matrix (1403) that causes them to float on the stomach contents. Upon ingestion, the immediate release (uncoated) microbeads release a first pulse of bucillamine, which is quickly absorbed through the stomach wall, while the coated microbeads float to the surface, releasing a second pulse of bucillamine about four, about five, or about six hours later, e.g., following exposure to the stomach environment and dissolution of the matrix coating. One of skill in the art will readily envision variations of this arrangement in view of the present teachings. For example, in some embodiments, the interval between periodic releases may be more or less than about four, about five, or about six hours, varying within a critical range, as taught herein. In some embodiments, additional sets of microbeads may be used to achieve additional pulses, e.g., a third, fourth, fifth, and/or sixth pulse, released at critically-spaced intervals in accordance with aspects of the invention. In some embodiments, a second multiple unit dosage form may be use, to provide, e.g., third and fourth pulses, where the second dosage form is swallowed about the same time as the first. Further, a different modified-release strategy may be employed, e.g., where the different sets of microbeads comprise modified-release components comprising different matrix coatings, reservoirs surrounded by matrices of different thicknesses, differently-releasing plugs, differently-expanding osmotic-push compartments, and the like, and any combinations thereof.

FIG. 14B depicts a single unit capsule (1404), shown in cross section, comprising an outer layer of immediate release bucillamine (1405), that coats a buoyant polymer matrix (1406), which itself encapsulates a core of bucillamine (1407). In this example, the bucillamine is released from the outer layer (immediate-release compartment), upon ingestion and entry into the low pH environment of the stomach, to provide a first pulse of drug at a first time point. After exposure to the stomach environment, the buoyant polymer matrix dissolves or disintegrates to provide a second pulse of drug about four, about five, or about six hours later. One of skill in the art will readily envision variations of this arrangement in view of the present teachings. For example, in some embodiments, the interval between periodic releases may be more or less than about four, about five, or about six hours, varying within a critical range, as taught herein. Further, a different modified-release strategy may be employed, e.g., where the core comprises modified-release components, such as alternating layers of bucillamine, polymer, and bucillamine, to achieve additional pulses, a matrix comprising differently-dissolving segments or differently-releasing polymers, a capsule comprising differently-dissolving bands, differently-releasing plugs, or differently-expanding osmotic-push compartments, drug reservoirs surrounded by matrices of different thicknesses, and the like, and any combinations thereof.

In some embodiments, the gastro-retentive floatation system comprises an effervescent agent to lower density and increase buoyancy. Effervescent delivery systems generally use stomach acid to combine with another molecule, generating carbon dioxide, which then causes a stretchable layer or coating to expand, lowering density of the dosage form, and allowing it to float to the stomach fluid surface, like a “bubble.” Examples of density-lowering effervescent agents for generating carbon dioxide include, but are not limited to, NaCO₃, bicarbonates, citric acid, or tartaric acid. Examples of polymers for use in a stretchable layer or coating include, but are not limited to, IPMC, ethyl cellulose, sodium alginate, or chitosan. The floating structure can remain on the fluid surface, where the polymers slowly dissolve to release drug, e.g., where the drug diffuses out in critically-spaced pulses over time, as taught herein.

Additional effervescent agents include volatile liquids that gasify at body temperature to cause inflation, in the stomach, e.g., expanding a liquid-containing compartment or chamber of the formulation. Suitable volatile liquids include, but are not limited to, ether and cyclopentane. Additional effervescent agents include an inert gas, e.g., trapped in a microporous compartment enclosing a drug reservoir.

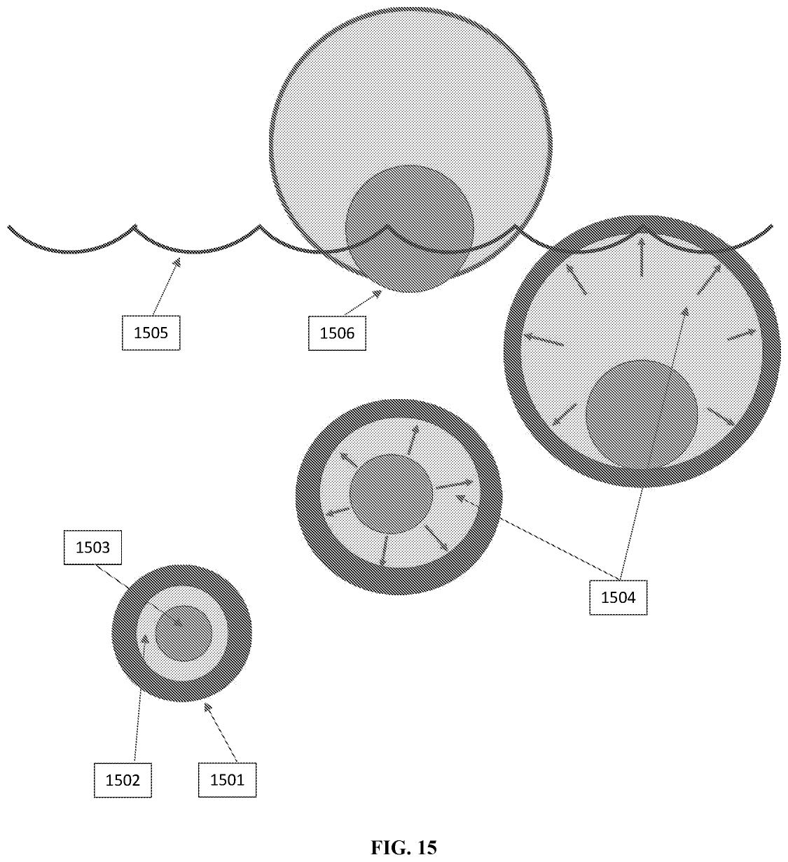

FIG. 15 depicts one example of a gastro-retentive floatation system comprising an effervescent agent. This example uses a bead, shown in cross section, comprising a layer of a stretchable polymer (1501), surrounding an NaCO₃ layer (1502), that itself surrounds a core of bucillamine (1503). Stomach acid permeates the stretchable polymer layer, reaching the NaCO₃ and causing release of carbon dioxide (1504). The carbon dioxide expands and causes the bead to rise to the top of the gastric fluids (1505). After a period of time, bucillamine is released (1506) into the stomach, due to dissolution of the polymer layer, or diffusion of bucillamine through it.

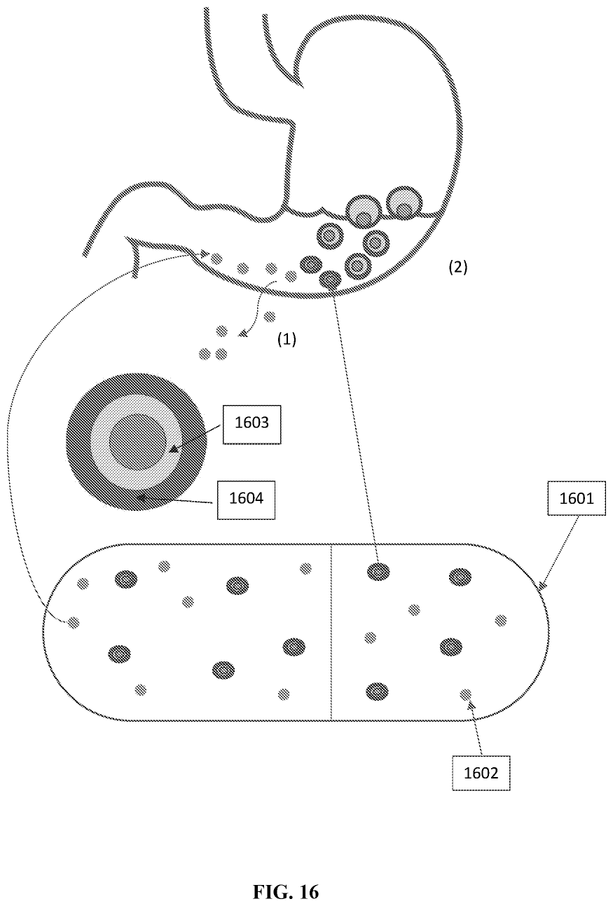

FIG. 16 depicts one example of a gastro-retentive floatation system comprising an effervescent agent that further comprises an immediate-release component. This example uses a capsule (1601), shown in cross section, comprising immediate release bucillamine beads (1602), and beads comprising bucillamine surrounded by an NaCO₃ layer (1603) that itself is surrounded by a stretchable polymer layer (1604). Upon ingestion and rapid dissolution of the capsule in the stomach, immediate release bucillamine is released and absorbed through the stomach at a first time point (1); while the coated bucillamine is released at a second time point (2), about four, about five, or about six hours later, as described above. One of skill in the art will readily envision variations of this arrangement in view of the present teachings. For example, in some embodiments, the interval between periodic releases may be more or less than about four, about five, or about six hours, varying within a critical range, as taught herein.

Additional gastro-retentive features known in the art also may be used in certain embodiments of the invention. See, e.g., Mandal, et al., 2016, “Gastro-retentive drug delivery systems and their in vivo success: A recent update,” Asian Journal of Sciences II, 575-584; Pant, et al., 2016, “A review on gastro retentive drug delivery system,” Indian Journal of Pharmaceutical and Biological Research, 4(2):1-10; Badul, et al., 2012, “Stomach specific drug delivery systems: A review,” International Journal of Pharmaceutical Research and Development, 4(04): 147-160; Yadav, et al., 2011, “Formulation development and in vitro characterization of bilayer and floating bioadhesive tablets of propranolol hydrochloride,” Asian J Pharmacy Life Sci. 1: 2-12; and Rajinikanth, et al., 2009, “Stomach-Site Specific Drug Delivery System of Clarithromycin for Eradication of Helicobacter pylori,” Chem. Pharm. Bull., 57(10): 1068-1075.

The modified-release components described herein, including gastro-retentive features, are used to improve pharmacokinetics of bucillamine in treating cystinuria, or a related disorder, or an inflammatory condition such as gout or rheumatoid arthritis. In a particular preferred embodiment, the approach used achieves four pulses of drug in the stomach, at critically-spaced intervals according to aspects of this invention. In some embodiments, the approach used achieves three pulses of drug in the stomach and one in the duodenum, at critically-spaced intervals. In some embodiments, the approach used achieves two pulses of drug in the stomach and two in the duodenum, at critically-spaced intervals. In some embodiments, the approach used achieves two pulses of drug in the stomach, one in the duodenum, and one in the ileum or jejunum, at critically-spaced intervals. In some embodiments, the approach used achieves one pulse of drug in the stomach, one in the duodenum, one in ileum or jejunum, and one in the large intestine, at critically-spaced intervals, according to aspects of this invention.

Enteric-Coated Delivery

In another approach regarding modified-release, the compositions comprise a coating, encapsulating the therapeutic agent, to achieve a delayed or a prolonged release of the therapeutic as the composition travels through the gastrointestinal tract. In particular embodiments, the coating delays release past the stomach, allowing increased uptake in the small intestines compared to the non-coated therapeutic. That is, in some embodiments, the extended-release approach prevents, substantially prevents, or reduces stomach uptake, leaving more of the therapeutic agent available for small intestine uptake, and then extends the period of release in the small intestine, preferably in repeated, critically-spaced pulses over a period of time, as described above. Increased uptake in the small intestine thus can refer to preferred or selective release in the small intestine, such as that brought about by certain embodiments of the modified-release strategies described herein.

In preferred embodiments, delayed release is followed by prolonged and controlled release, for example, release of the therapeutic as the composition travels through the small intestines, preferably in repeated, critically-spaced pulses over a period of time, as described above. Controlled-release of bucillamine, for example, extends the duration of therapeutic effect of the drug, as small amounts are released over an extended period of time, preferably in critically-spaced pulses. In some embodiments, the enteric-coated formulations of the invention reduce toxicity of bucillamine, reducing severity and/or incidence of adverse side effects. In some embodiments, the enteric-coated formulations of the invention improve therapeutic efficacy of bucillamine, for example, in reducing urinary cystine concentrations. In some embodiments the reduction in toxicity and/or increase in efficacy is statistically significant compared to immediate-release (or delayed-release) versions.

In some embodiments, the invention provides a composition comprising a coated therapeutic agent that has increased uptake in the small intestine compared to the non-coated therapeutic agent when administered orally. Enteric-coated formulations of the invention release the therapeutic agent, e.g., bucillamine, upon dissolution or disintegration of a coating, typically a coating encapsulating a core of the therapeutic agent. In some embodiments, the therapeutic agent is enterically coated. An “enterically coated” drug refers to a drug that is coated with a substance, i.e., with a coating that remains intact, or substantially intact, in the stomach but dissolves or disintegrates to release the therapeutic agent once the small intestine is reached. An “enteric coating” refers to a material, usually one or more polymeric materials, that encases a core region containing the therapeutic agent (e.g., encasing bucillamine) for use in the modified-release formulations described herein, where the material remains intact, or substantially intact, in the stomach and then dissolves or disintegrates to release the therapeutic agent in a prolonged and/or controlled manner, over an extended period of time, generally over several hours, preferably in repeated, critically-spaced pulses, as described above.

In particular embodiments, enteric coating materials are selected such that the therapeutic agent is not released until the composition reaches the small intestine, or a region in which the pH is greater than pH 4.5. A suitable pH-sensitive material is one which will dissolve in intestinal juices at a higher pH than that typically found in the upper portion of the gastrointestinal tract, like the stomach, such that disintegration or dissolution occurs in the small intestine. In some embodiments, the pH-sensitive material does not undergo significant disintegration or dissolution until the dosage form has emptied from the stomach, such as after passing the pyloric sphincter. For example, the extended-release coating material may begin to dissolve in an aqueous solution at pH between about 4.5 to about 5.5, permitting release of the therapeutic agent beyond the stomach and in regions of the beginning of the small intestine.

In some embodiments, the selected coating materials begin to dissolve within the pH range of the upper part of the small intestines, such as the duodenum, and then different polymeric materials sequentially disintegrate or dissolve over increasing pH, ranging from about 4.5 to about 7.2, allowing release of the therapeutic agent at increasing pH ranges corresponding to the increase in pH along the jejunum and towards the ileum. The amount and types of enteric coatings, e.g., different polymer coatings, are selected to substantially dissolve at increasing pH over the approximate three hour transit time through the small intestine.

In some embodiments, the enteric coatings are selected and arranged so as to release repeated, critically-spaced pulses over time, once past the stomach, e.g., according to any of the examples described in FIG. 3-FIG. 8, above. For example, any of the modified-release components described herein, or variations thereof, may further be designed for small intestine delivery of bucillamine, e.g., by enclosing the pill, tablet, capsule, etc. in an enteric coating, and/or by omitting stomach-dissolving or stomach-releasing elements of the modified-release components.

In some embodiments, e.g., the modified-release component of FIG. 3 excludes the outermost layer of bucillamine (304); the modified-release component of FIG. 4 excludes the first reservoir (403) released in the stomach upon dissolution of the relatively thin matrix segment (404) surrounding it; the modified-release component of FIG. 5 excludes the first set of uncoated beads (504); in the modified-release component of FIG. 6B, the first band of capsule (604) dissolves to release a first pulse of bucillamine (605) beyond the stomach; in the modified-release component of FIG. 7, the cap (704) comprises an enteric-coating to delay release of a first pulse (bolus) of bucillamine (705) beyond the stomach; and the modified-release component of FIG. 8B excludes the first packet (805) enclosed by a first coating (806) that first dissolves in the stomach.

For compositions comprising bucillamine, the extended-release coating may comprise a polymeric material that prevents release of the bucillamine in the low pH environment of the stomach, but that ionizes at a slightly higher pH, typically at a pH of about 4 or about 5, and thus dissolves or disintegrates sufficiently in the small intestines to begin releasing bucillamine. In some particular embodiments, the extended-release coating comprises one or more polyacids having a pKₐ in the range of about 3 to about 5. Particular examples of suitable materials for extended-release coatings include polymerized gelatin, shellac, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), phthalic acid methylcellulose (CMP), hydroxypropyl methylcellulose phthalate, starch acetate (SAP), alginic acid and its calcium salt, pectin, and a calcium salt, hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit NM, Eudragit RL, Eudragit RS). For example, in one embodiment, the extended-release coating comprises Eudragit L30D, triethylcitrate, and hydroxypropylmethylcellulose (HPMC), and either the (+) or (−) enantiomer of bucillamine, and the coating comprises less than about 15% of the composition. In some embodiments, the modified-release formulations of the present invention do not comprise bucillamine that is covalently attached to the polymer materials used to effect extended release. For example, in some embodiments, the modified-release formulations of the present invention do not comprise polyanhydride compositions described in U.S. Pat. No. 6,613,807, to Uhrich.

Microbead Delivery

In a particular embodiment, a capsule comprising a plurality of microbeads is used, wherein the plurality of microbeads comprises different populations having coatings of different thicknesses and/or different materials and/or different proportions of the same or of different materials. For example, the pharmaceutical composition may comprise a first population of microbeads having a coating of a first thickness or first thickness range, comprising one or more suitable materials, to allow dissolution and release of a first amount of the therapeutic, e.g., bucillamine, when the composition reaches the stomach. In some embodiments, the composition comprises a second population of microbeads having a coating of a second thickness, or second thickness range, comprising one or more suitable materials, to allow dissolution and release of a second amount of the therapeutic, e.g., bucillamine, at a second critically-spaced time point, and/or as the composition travels along the duodenum. In some embodiments, the composition comprises a third population of microbeads having a coating of a third thickness, or third thickness range, comprising one or more suitable materials, to allow dissolution and release of a third amount of the therapeutic, e.g., bucillamine, at a third critically-spaced time point and/or as the composition travels along the jejunum. In some embodiments, the composition comprises a fourth population of microbeads having a coating of a fourth thickness, or fourth thickness range, comprising one or more suitable materials, to allow dissolution and release of a fourth amount of the therapeutic, e.g., bucillamine, at a fourth critically-spaced time point and/or as the composition travels along the ileum. As one of skill in the art will appreciate, the coating thickness of the first, second, third, and fourth populations increases in that order. The invention contemplates use of multiple thicknesses, e.g., 2, 3, 4, 6, 8, or 10, different thicknesses, or thickness ranges of various populations of microbeads, thereby allowing incremental, repeated release of the therapeutic, e.g., in critically-spaced pulses as described herein.

In some embodiments, differences in coating thickness is achieved by designing an asymmetrical coating, e.g., where the coating is thicker at one end (or side) of a capsule than the other. Coating thickness may increase gradually from one end/side to the other, or more preferably, coating thickness may increase in steps, e.g., in steps to allow dissolution and release of first, second, third, and fourth amounts of the therapeutic, e.g., bucillamime, in critically-spaced pulses as described herein, e.g., as the composition reaches the stomach, passes the stomach, travels the duodenum, travels the jejunum, and travels the ileum, or while the composition resides in the small intestine, more preferably while the composition resides in the stomach.

Topical Delivery

In one approach, the pharmaceutical composition is formulated for topical delivery, e.g., in transdermal or transmucosal formulations. Transdermal formulations or systems generally comprise a therapeutically effective amount of bucillamine for transdermal delivery and a pharmaceutically acceptable modified-release component that achieves pulsed release of the drug, in accordance with the present disclosures, through the skin upon topical application of the formulation. One of skill in the art will envision various topical formulation designs and/or arrangements that bring about the desired release schedule.

In some embodiments, the modified-release component of the transdermal delivery system comprises reservoirs of different sizes, and/or different permeabilities, to release bucillamine at step-wise, critically-spaces times in repeated pulses, in accordance with aspects of the invention. For example, the drug may be held in reservoirs of different sizes, with an impermeable backing on one side and a membrane on the opposite side that allows drug to cross, at different rates, to contact the skin.

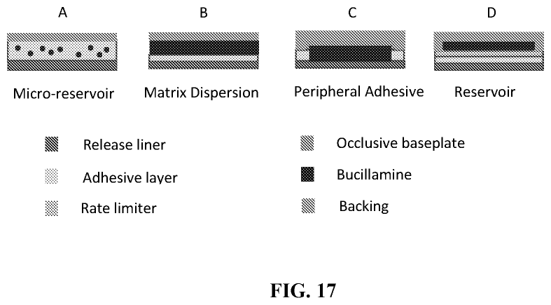

FIGS. 17A-17D depict particular examples of transdermal delivery systems with modified-release components, including a system comprising micro-reservoirs (FIG. 17A), a matrix dispersion (FIG. 17B), a peripheral adhesive (FIG. 17C), and a single reservoir (FIG. 17D). One of skill in the art will readily envision variations of these arrangements in view of the present teachings. For example, in some embodiments, two or more of these systems are combined to achieve, e.g., second, third, fourth, fifth, sixth, seventh, and/or eight pulses, released across the skin at critically-spaced intervals in accordance with aspects of the invention.

In a particularly preferred embodiment, the transdermal delivery system is provided in a skin patch (or dermal patch). The skin patch may comprise embedded drug along with one or more microneedles to aid delivery of bucillamine past the stratum corneum layer of the skin and into the dermis. Microneedle patches can deliver boluses of drug over time, using a mixed needle array. For example, the mixed needle array may comprise various microneedle types, some delivering drug immediately, some over an intermediate time, and some over a longer period of time, to release bucillamine at step-wise, critically-spaced times in repeated pulses, in accordance with aspects of the invention.

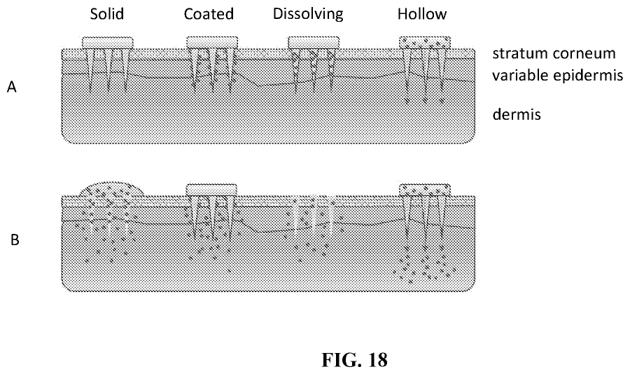

FIGS. 18A-18B depict particular examples of transdermal delivery systems with modified-release components comprising microneedles, including systems with solid, coated, dissolving, or hollow microneedles. FIG. 18A depicts the systems before release; FIG. 18B depicts the systems during and after release of the drug. Where a solid microneedle is used, the microneedle creates holes in the top layers of skin into which drug is applied (as in a gel, cream, or lotion), increasing the permeability of skin and allowing the drug to penetrate. Where a coated microneedle is used, drug can be embedded on the exterior of microneedle array; once placed on the skin, drug migrates off the needle and into the skin. Where a dissolving microneedle is used, the needle may be made of various polymers, such as PLA, PGA, PLGA, PVP, and polycarbonate, which degrade over time, releasing embedded drug. Where a hollow microneedle is used, the drug is allowed to flow from a reservoir through the needle and through the skin. One of skill in the art will readily envision suitable variations of these arrangements in view of the present teachings. In a particular embodiment, the skin patch comprises differently-dissolving microneedles, where the different microneedles dissolve at step-wise, staggered times, to release bucillamine across the skin at critically-spaced intervals, in accordance with aspects of the invention.

Other transdermal approaches that may be used with the compositions and delivery systems disclosed herein include, e.g., thermal poration, hypodermic needles, iontophoresis, electroporation, ultrasound, sonophoresis, jet injection, or a combination thereof (Prausnitz et al. 2004, Nature Reviews Drug Discovery 3:115-124)). See also, WO 96/11705 and WO 89/04179, describing formulations containing plaster and the absorption promoter dimethylisosorbide.

The dermal patch may be applied to any suitable area of the subject's skin, such as but not limited to, the upper arm, axilla, shoulder, forearm, back of the head, chest, back, abdomen, buttocks, hip, thigh, behind the ear, neck, face, eye area, wrists, ankles, joints, finger joints, toe joints, and the like, or any combination thereof. Typically, the patch is applied to a clean, dry area of skin, more preferably to a hairless area or a relatively hairless area.

In some embodiments, the pharmaceutical composition is formulated for buccal or sublingual delivery or delivery across other mucosal membranes. Buccal/sublingual formulations or systems, e.g., generally comprise a therapeutically effective amount of bucillamine for transmucosal delivery and a pharmaceutically-acceptable modified-release component that achieves pulsed release of the drug, in accordance with the present disclosures, through the oral mucosa upon application to a surface within the mouth cavity. One of skill in the art will envision various buccal formulation designs and/or arrangements to bring about the desired release schedule.

Preparation and use of compositions and delivery systems of the present invention are described in further detail in the sections below.

Preparation of Modified-Release Compositions and Systems

Another aspect of the present invention relates to methods of preparing the modified-release compositions and systems described herein. Generally, the modified-release formulations of the present invention are provided as pharmaceutical compositions, comprising a the pharmaceutically-acceptable modified-release component, a suitable dose of the therapeutic, and a pharmaceutically acceptable carrier.

The pharmaceutical compositions can be prepared by mixing the therapeutic, e.g., bucillamine, with one or more pharmaceutically acceptable carriers, such as pharmaceutically acceptable diluents, vehicles, or excipients. In preferred embodiments, the modified-release formulations are for oral administration and the pharmaceutically acceptable carrier is a pharmaceutically acceptable diluent, vehicle, or excipient suitable for oral administration. In this context, the term “pharmaceutically acceptable carrier” refers to a material suitable for oral administration and not biologically, or otherwise, undesirable, i.e., a material that may be administered to a subject along with the therapeutic agent without causing undesirable biological effects, or undesirable side effects that outweigh the benefits of the therapeutic agent, and/or that does not interact in a deleterious manner with the therapeutic or with other components of the formulation or system in which it is contained. For topical delivery, e.g., using transdermal or transmucosal formulations, the “pharmaceutically acceptable carrier” is a pharmaceutically acceptable diluent, vehicle, or excipient suitable for transdermal or transmucosal administration, i.e., a material that may be administered to the skin (or oral mucosa or other mucosal surface) of a subject along with the therapeutic agent without causing undesirable biological effects, or undesirable side effects that outweigh the benefits of the therapeutic agent.

A “pharmaceutically acceptable modified-release component,” such as a “pharmaceutically acceptable coating” or a “pharmaceutically acceptable matrix,” refers to a component comprising materials suitable for oral, transdermal, or buccal administration, or other suitable administration, that is not biologically, or otherwise, undesirable, i.e., a material that may be administered to a subject by the desired route, along with the therapeutic agent, without causing undesirable biological effects, or undesirable side effects that outweigh the benefits of the therapeutic agent, and that does not interact in a deleterious manner with the therapeutic agent or with other components of the formulation or system.

In pharmaceutical compositions, the therapeutic agent may be provided as a pharmaceutically acceptable salt, ester, or other derivative, comprising, for example, salts, esters, or other derivatives of the therapeutic agent that are not biologically or otherwise undesirable, or whose undesirable side effects do not outweigh the benefits of the therapeutic agent. For example, bucillamine may be administered in the form of a pharmacologically acceptable salt, ester, amide, prodrug, or analog, or as a combination thereof. Salts, esters, amides, prodrugs and analogs of the therapeutic agents may be prepared using standard procedures known to those skilled in the art of synthetic organic chemistry and described, for example, by J. March, “Advanced organic Chemistry: Reactions, Mechanisms and Structure,” 4th Ed. (New York: Wiley-Interscience, 1992). For example, to prepare basic addition salts from the neutral drug, a reaction of one or more of the therapeutic agent's free hydroxyl groups can be reacted with a suitable base. The neutral form of the drug can be dissolved in a polar organic solvent, such as methanol or ethanol, and base then added. The resulting salt either precipitates or may be brought out of solution by adding a less polar solvent. Suitable bases for forming basic addition salts include, but are not limited to, inorganic bases such as ammonium hydroxide, calcium hydroxide, potassium hydroxide, sodium hydroxide, trimethylamine, and the like. Preparation of esters involves functionalization of hydroxyl groups present in the drug. Typically, esters are acyl-substituted derivatives of free alcohol groups, i.e., moieties which derived from carboxylic acids of the formula R—COOH where R is an alkyl, typically a lower alkyl. Esters can be reconverted to the free acids, if desired, by using conventional procedures, such as hydrogenolysis or hydrolysis. Preparation of amides and prodrugs can be carried out in a similar manner. Other derivatives and analogs of the therapeutic agents may be prepared using standard techniques known to those skilled in the art of synthetic organic chemistry, or may be deduced by reference to relevant literature.

Dosage Form Preparation

The compositions of the present invention are formulated in various dosage forms, generally depending on the desired mode of administration. In preferred embodiments, the composition is formulated for oral administration and the dosage form is a tablet, pill, powder, troche, lozenge, minitablet, or capsule, for example, a tablet or capsule providing a total therapeutically effective amount of bucillamine for pulsed release over an extended period of time, as described herein.

In some embodiments, tablets are manufactured by first coating the therapeutic agent, e.g., bucillamine. One method for forming tablets involves direct compression of powders containing a coated bucillamine, optionally in combination with diluents, binders, lubricants, disintegrants, colorants, stabilizers, and the like. As an alternative to direct compression, compressed tablets can be prepared using wet-granulation or dry-granulation processes. Tablets may also be molded rather than compressed, starting with a moist material containing a suitable water-soluble lubricant. Different coatings can be used for different layers of a given tablet or for different sets of tablets, e.g., to give one or more of the designs described herein, or variations thereof.

In some embodiments, a coated bucillamine is granulated and the granulation is compressed into a tablet or filled into a capsule. Capsule materials may be either hard or soft, and are typically sealed, such as with gelatin bands, or the like. In some embodiments, different coatings are used in different sets of beads for filling a given capsule, or for filling different sets of capsules; and/or the capsule material itself is composed of different segments, e.g., to give one or more of the designs described herein, or variations thereof.

Tablets and capsules for oral use will generally include one or more commonly used excipients. Nonlimiting examples of excipients include binders, bulking agents, diluents, disintegrants, lubricants, fillers, and the like, combined with the therapeutic agent (bucillamine) in the composition. Diluents are typically necessary to increase the bulk of the dosage form (e.g., a tablet or capsule) so that a practical size is provided for compression. Suitable diluents include dicalcium phosphate, calcium sulfate, lactose, cellulose, kaolin, mannitol, avicel, sodium chloride, dry starch, and powdered sugar.

Some embodiments use Neusilin® as an excipient. Neusilin® is a magnesium aluminummetasilicate, which improves tableting low molecular drugs. Neusilin® can be used in direct compression and wet granulation in formulating solid dosage forms, e.g., in tablets, powders, granules, and/or capsules. In a particular embodiment, Neusilin® helps to neutralize unpleasant odors that may be caused by the thiol groups of bucillamine. Other approaches for use in reducing unpleasant orders of pharmaceutical compositions also may be used with the modified-release compositions described herein (see, e.g., WO 2014/080365, describing use of a melt-processable polymer in a composition prepared by hot-melt extrusion).

In some embodiments, oral dosage forms also include binders, fillers, and/or lubricants. Binders can be included to impart cohesive qualities to a tablet formulation, helping the tablet remain intact after compression. Nonlimiting examples of materials suitable for use as binders include starch (including corn starch and pregelatinized starch), gelatin, sugars (including sucrose, glucose, dextrose and lactose), polyethylene glycol, e.g., PEG400, waxes, and natural and synthetic gums, e.g., acacia sodium alginate, polyvinylpyrrolidone, cellulosic polymers (including hydroxypropyl cellulose, hydroxypropyl methylcellulose, methyl cellulose, hydroxyethyl cellulose, and the like), and Veegum. In some embodiments, fillers are used that may be insoluble materials, such as silicon dioxide, titanium oxide, alumina, talc, kaolin, powdered cellulose, microcrystalline cellulose, and the like. Additionally or in the alternative, soluble fillers may be used, such as mannitol, urea, sucrose, lactose, dextrose, sodium chloride, sorbitol, and the like. In some embodiments, lubricants are used to facilitate tablet manufacture. Nonlimiting examples of materials suitable for use as lubricants include magnesium stearate, calcium stearate, stearic acid, and the like. Lubricant materials generally make up less than about 1% by weight of the final dosage form (e.g., of the final tablet).

In some embodiments, dosage forms for oral administration include one or more disintegrants, that facilitate disintegration or “breakup” of the tablet or capsule after administration, preferably step-wise, at critically-spaced time points, as described herein. Nonlimiting examples of materials suitable for use as disintegrants include starches, clays, celluloses, algins, gums, cross-linked polymers, and the like.

Dosage forms for oral administration also may contain minor amounts of non-toxic auxiliary substances, such as wetting or emulsifying agents, pH buffering agents, and the like. Nonlimiting examples include sodium acetate, sorbitan monolaurate, triethanolamine sodium acetate, and triethanolamine oleate. In some embodiments, flavoring, coloring, and/or sweetening agents may be added as well. In some embodiments, the oral formulation may further comprise one or more preservatives, suspending agents, thickening agents, and the like.

In some embodiments, the pharmaceutical composition for modified-release of bucillamine is stabilized. The composition may be stabilized by including one or more stabilizing agents in an oral formulation. “Stabilizing agents” refer to compounds that lower the rate at which the bucillamine degrades, particularly for oral dosage forms under typical conditions of storage. Nonlimiting examples of materials suitable for use as stabilizing agents include hydroxypropyl methylcellulose; polyvinylpyrrolidone; cellulosic polymers such as hydroxypropyl cellulose, hydroxyethyl cellulose, methyl cellulose, ethyl cellulose, cellulose acetate, cellulose acetate phthalate, cellulose acetate trimellitate, hydroxypropyl methylcellulose phthalate, microcrystalline cellulose, and carboxymethylcellulose sodium; vinyl polymers and copolymers, such as polyvinyl acetate, polyvinylacetate phthalate, vinylacetate crotonic acid copolymer, and ethylene-vinyl acetate copolymers; and the like. See also, U.S. Pat. No. 4,301,146. The stabilizing agent generally is present in an amount effective to provide the desired stabilizing effect. The stabilizing agent may be present in a ratio of bucillamine to stabilizing agent of at least about 1:500 w/w, about 1:400 w/w, about 1:200 w/w, or about 1:100 w/w. In some embodiments, the stabilizer is an antioxidant, e.g., oil-soluble antioxidants (e.g., butylated cresol, butylated hydroxytoluene, vitamin E, and the like); water-soluble antioxidants (e.g., methionine, sodium bisulfite, metabisulfite, sodium metabisulfite, sodium sulfite, sodium thiosulfate, thiourea, vitamin C, and the like); tert-butyl-hydroxy anisole, and the like.

In some embodiments, the bucillamine component further comprises one or more penetration enhancers, e.g., penetration enhancers designed to facilitate uptake at a particular region of the gastrointestinal tract, preferably the stomach. Examples of additional ingredients include plasticizers such as triethyl citrate, acetyltrietylcitrate, diethyl sebacate, dibutyl sebacate; polymers such as carbomer, chitosan, chitosan cysteine, sodium carboxymethyl cellulose, N-trimethylated chitosan, polycarbophil-cysteine, long chain fatty acids, their esters (for example, mono and diglycerides) and their salts such as lauric acid, sodium lauryl sulfate, palmitic acid, caprylic acid, capric acid, oleic acid, acylcamitine; chelating agents such as metal chelating agents, disodium edetate, citric acid, tartaric acid, EDTA, salicylates, cyclodextrins, polyacrylic acids; bile acids such as cholic acid, cholyltaurine, cholylsarcosine, chenodeoxycholic acid and its salts such as sodium cholate, sodium glycocholate, sodium taurocholate, sodium taurodihydrofusidate; surfactants and emulsifiers such as in particular polyethylene 660-12-hydroxy-stearate (Solutol® HS15) (Solutol HS15), polysorbate 80 (Tween 80), polyoxyethylated castor oil (Cremophor EL) Polyoxyethylene-polyoxypropylene glycol (Pluronic® F68), zonula occludens toxin, the toxin (ZOT); vitamins, such as vitamin E (tocopherol), vitamin B12, and the like or any combination thereof.

Some embodiments use minitablets as the dosage form. A “minitablet” is a compressed tablet that typically has a diameter of about 1-4 mm. In a particular embodiment, the minitablet is formulated to contain an appropriate dose per weight of patients in a capsule, providing patient-centric dosage forms, which may further encourage patient compliance. For example, minitablets are particularly useful for pediatric and veterinary uses. In some embodiments, the compositions are provided in the form of pharmaceutically acceptable microcapsules (see, e.g., expired U.S. Pat. No. 6,428,804 to Sukuzi, entitled “Intra-articular preparation for the treatment of arthropathy”), nanocapsules (see, e.g., EP Patent 1010435, entitled “Nanocapsule preparations for treating intraarticular diseases”), nanoparticles, nanospheres, and nanoformulations (see, e.g., Delie et al, 2015, Molecule, 10:65-80). In some embodiments, the compositions are provided in the form of microparticulates, e.g., microparticulates ranging from about 10 μm to about 2 mm (e.g., described in U.S. 2010/0310541 to Kessler et al, entitled “Compositions and Methods for Reducing the Toxicity of Certain Toxins, e.g., ¶¶ [0100]).

In some embodiments, the composition is formulated for topical administration and may be provided as a cream, ointment, lotion, gel, paste, spray, powder, suppositories, emulsions, aerosols, dispersion, or any other form typically used for topical or transmucosal application. In a particular embodiment, the composition is provided as a gel with a therapeutically effective concentration of bucillamine for pulsed release over an extended period of time, as described herein, upon application to the skin or a mucosal surface.

The formulation for topical application may contain one or more materials typically used in dermatological formulations. For example, in some embodiments, the topical formulation includes one or more permeability enhancers, such as but not limited to, azone (1-dodecylazacycloheptan-2-one) and SEPA (2-n-nonyl-1,3dioxolane). In some embodiments, the topical formulation comprises a solvent or co-solvent, with or without a permeability enhancer, and/or with an emollient, spreading agent, and/or film-forming agent. Emollients, spreading agents, and/or film-forming agents for use in topical formulations include, but are not limited to, polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils. Other ingredients may include anionic surfactants, like sodium, potassium, or ammonium stearates, calcium stearate, triethanolamine stearate, sodium abietate, alkyl sulphates, sodium dodecylbenzenesulphonate, sodium dioctylsulphosuccinate; cationic surfactants, such as water-soluble quaternary ammonium salts or cetyltrimethylammonium bromide; amine salts such as octadecylamine hydrochloride; nonionic surfactants like sorbitan esters, which are optionally polyoxyethylenated (e.g., polysorbate 80), polyoxyethylenated alkyl ethers, polyoxypropylated fatty alcohols such as polyoxypropylene-styrol ether, polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide; amphoteric surfactants; and the like, or any combination thereof.

Organic solvents that can be used include but are not limited to: acetyltributyl citrate, fatty acid esters like dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g., N-methylpyrrolidone), diethylene glycol monoethyl ether, ethylene glycol, diethyl phthalate, and the like, or any combination thereof.

Delivery System Preparation

Delivery systems of the invention further provide the pharmaceutical compositions incorporated within structures or devices that effect release at step-wise, critically-spaced times to provide repeated pulses of the therapeutic over time, in accordance with aspects of the invention. For example, an oral formulation may be incorporated in a delivery system comprising matrixes, beads, liposomes, vesicles, microcapsules, microspheres, solid particulate materials, dispersions, microfluidic devices, microprocessors, and the like, as described herein. For example, in some embodiments, a layered tablet structure is used that can release the drug over time in the stomach, in a step-wise fashion, as described herein. In some embodiments, microbeads of bucillamine are coated and the coated beads contained within a capsule, for release in the stomach, over time in a step-wise fashion, e.g., as described herein. In some embodiments, the drug is embedded in a waxy matrix, which releases the drug in the stomach, over time in a step-wise fashion, e.g., again as described herein, and the matrix subsequently is excreted in the patient's feces. One of skill in the art will readily be able to construct such delivery systems based on technology in the art and the disclosures herein.

In some embodiments, the drug is incorporated with a gastro-retentive feature, such as coating with a stomach-adhering polymer, combining or coating with a high density agent, embedding in a swellable or shape-modifying structure, such as a star-shaped structure folded into a capsule, coating with buoyant polymers, combining with an effervescent agent and coating with a stretchable polymer, and the like. One of skill in the art will readily be able to construct such delivery systems based on technology in the art and the disclosures herein.

In some embodiments, bioadhesive polymers, such as stomach-adhering polymers, are used in conjunction with various technologies to form articles that bind mucosal or epithelial membranes. For example, bioadhesive microspheres may be formed by dispersing carboxyvinyl polymer powder in waxy hydrogenated castor oil. (See, e.g., Sanap, 2009, “Formulation and evaluation of mucoadhesive beads of glipizide using 23 factorial designs,” J Pharmacy Res, 2: 934-938). In some embodiments, a bioadhesive formulation is mixed with sodium alginates, carbapol, or sodium carboxy methyl cellulose; and/or including one or more excipients, such as but not limited to, polycarbophil, carbopol, lectins, chitosan, CMC and gliadin.

Other drug delivery systems that may be used to effect critically-spaced, repeated release of bucillamine, in accordance with the invention, include, e.g., those described in WO 2004/060346 A3 (disclosing a rapid gelling polymer composition for drug delivery); U.S. 2005/0025807 (disclosing porous calcium phosphate materials used to release drug); WO 2007/044693 (disclosing ionic liquid compositions used for controlled release of drugs); and WO 2007/087256 and U.S. 2016/0206746 (disclosing dendritic structures that use biocompatible linkers and biodegradable bonds, such that drugs may be incorporated into the structure and released sequentially and quantitatively upon degradation or digestion of the bonds); ion exchange resins (see, e.g., U.S. 2007/0043120); and segments of a dosage form of different breaking strengths (see, e.g., WO 2014/191396).

A topical formulation may be incorporated in a delivery system suitable for application to the skin or mucosal surface, such as a delivery system comprising reservoirs, matrices, microneedles, materials typically used in skin patches, and the like. One of skill in the art will readily be able to construct such delivery systems based on technology in the art and the disclosures herein.

For example, in some embodiments, a patch is designed to contain a bucillamine-containing pharmaceutical composition in a compartment, where the bucillamine is suspended or dissolved in solvent to form a liquid or a gel to give a liquid or gel reservoir. The liquid or gel reservoir may be separated from a continuous adhesive layer by a permeable membrane that controls the release of bucillamine from the device. In some embodiments, the bucillamine is incorporated into the adhesive backing of the patch without a distinct drug-containing reservoir. In some embodiments, various patch designs are used, including but not limited to, matrix patches, patches with active delivery systems, microneedle patches with cutaneous solutions, metered-dose systems, and the like, or any combination thereof (see, e.g., Pastore, et al., 2015, “Review—Transdermal patches: history, development and pharmacology,” British J. of Pharmacol 172: 2179-2209).

Manufacture of a given skin patch largely is determined by the type of patch. Reservoir patches generally are produced by a form-filling and sealing process or a coating-drying process (see, e.g., U.S. Pat. No. 3,797,494, to Zaffaroni, entitled “Bandage For The Administration Of Drug By Controlled Metering Through Microporous Materials;” and U.S. Pat. No. 4,460,372, to Campbell et al., entitled “Percutaneous absorption enhancer dispenser for use in coadministering drug and percutaneous absorption enhancer”).

In some embodiments, a microneedle array is used to effect transdermal delivery, generally in combination with a skin patch. In some embodiments, a microneedle array is used to puncture the skin, and then removed and replaced with a drug-containing patch. In some embodiments, the microneedle is coated with a solution, suspension, or gel comprising bucillamine and left in place for a period of time. In some embodiments, the microneedle array left in place comprises hollow-bore microneedles, through which a bucillamine-containing composition is allowed to diffuse or is driven by pressure through a central lumen.

Microneedle arrays may be manufactured by, e.g., lithography and etching, hot melts, micromolding, and the like. In some embodiments, micromolding of metals is used, such as but not limited to, silicon, stainless steel, titanium, palladium, palladium-cobalt alloys, nickel, and the like, and combinations thereof. In some embodiments, ceramic slurries are used, e.g., by being cast into a micromold. Biocompatible ceramics may comprise one or more of alumina, gypsum, brushite, and/or other materials currently used as bone cements. In some embodiments, biopolymers are used to form a matrix with bucillamine for casting into a micromold. Biopolymers include, but are not limited to, carboxymethlycellulose, amylopectin, dextrin, hydroxypropyl cellulose, alginate, hyaluronic acid, and the like, or a combination thereof.

Differently-dissolving and/or differently-biodegrading microneedles may be manufactured for use in certain embodiments of the invention. For example, in a preferred embodiment, an array of differently-biodegrading microneedles is constructed, where different sets of microneedles comprise different materials that biodegrade in skin layers, at step-wise, critically-spaced times, to release repeated pulses of bucillamine, as described herein. In a particularly preferred embodiment, an array of differently-dissolving microneedles is constructed, where different sets of microneedles comprise different polymer make-ups that dissolve at step-wise, critically-spaced times, to release repeated pulses of bucillamine over time, in accordance with aspects of the present invention. Biodegradable and/or polymeric microneedle arrays can be made by preparing slurries or hot melts of bucillamine and carbohydrates, which then can be cast into a micromold. Carbohydrates for use in slurries or hot melts with bucillamine, e.g., for biodegradable microneedles, include, but are not limited to, maltose, trehalose, sucrose, mannitol, xylitol, galactose, and the like, or combinations thereof. Polymers for use in slurries or hot melts with bucillamine, e.g., for dissolving microneedles, include, but are not limited to, poly(methyl methacrylate) (PMMA) polylactic acid, poly(carbonate), cyclic-olefin copolymer, poly(vinylpyrrolidone) (PVP), poly(vinylalcohol) PVA, polystyrene (PS) [97], poly(methyl vinyl ether-co-maleic anhydride), poly(methyl vinyl ether-co-maleic acid), and the like, or combinations thereof. See also, e.g., Larraneta, et al., 2016, “Review—Microneedle arrays as transdermal and intradermal drug delivery systems: Materials science, manufacture and commercial development,” Materials Sci. and Eng. R 104: 1-32.

Use of the modified-release pharmaceutical compositions, delivery systems, and dosage forms of the present invention is described in the sections below

Methods and Uses of Modified-Release Compositions and Systems

Still another aspect of the present invention relates to methods of using the modified-release compositions and systems described herein for delivering an effective amount of a therapeutic agent (bucillamine) to achieve an extended duration of effect following administration to a subject in need thereof. In particular, the present modified-release compositions find use in treating and/or preventing cystinuria, a disorder related thereto, or a symptom thereof. In further embodiments, the present extended-release compositions find use in treating and/or preventing one or more additional conditions where use of bucillamine is indicated, including inflammatory conditions, such as gout or a condition related thereto, or a symptom thereof, or rheumatoid arthritis, a condition related thereto, or a symptom thereof, or other forms of arthritis.

The “subject” referred to herein means a mammalian subject, including a human patient and mammals typically kept as pets, such as dogs, cats, hamsters, rabbits, mice, and the like. In particular, the subject will be a human patient or a dog, such as a pet dog with cystinuria. In preferred embodiments, the dog is a breed of dog known to affected by cystinuria, e.g., Bulldogs, Newfoundlands, and Labrador Retrievers. The mammalian subject also may be an animal typically farmed, such as cows, horses, donkeys, goats, sheep, pigs, and the like.

As discussed above, cystinuria patients accumulate cysteine and other amino acids in their urine, leading in the case of cysteine to formation and precipitation of cystine and painful cystine stones. Cystinuria patients face a life-long risk of stone formation, as well as impaired renal function and repeated surgeries, and require life-long treatment (Andreassen et al., 2016, “How should patients with cystine stone disease be evaluated and treated in the twenty first century?,” Urolithiasis, 44:65-76). Current tiopronin medications fail 85% of cystinuria patients, who continue forming stones, largely due to short duration of efficacy and uncontrolled, rising cystine levels in the urine overnight. Clinical trials with bucillamine fail to address reducing the frequency of dosing, as well as other problems typical of tiopronin therapy.

Delivery approaches of the present invention provide a strategy for improving pharmacokinetics of bucillamine to achieve higher efficacy, longer duration, and less adverse effects, using fewer administrations and/or lower total daily dosing. The modified-release bucillamine compositions and systems described herein can surprisingly provide clinically-superior treatment compared to immediate-release (or delayed-release) versions of the drug. In particular, dosage compliance is critical to efficacy in rare diseases, where patients require life-long treatment. In preferred embodiments, the modified-release formulations of the present invention allow less frequent administrations or applications, encouraging compliance. Additionally, whereas immediate release formulations fail to address cystine accumulation overnight in cystinuria patients, formulations described herein can allow better and more consistent cystine stone control, decreasing, and preferably preventing, cystine stone formation.

In particular embodiments, the present extended-release compositions deliver bucillamine in the stomach, over an extended period of time, which dramatically reduces peak plasma concentrations while achieving higher and more consistent urine concentrations of the drug, thereby reducing urinary cystine concentrations and consequent stone formation. The reduction in peak plasma concentrations can be statistically significant compared to immediate-release (or delayed-release) versions. The bucillamine delivery methods provided herein thus can surprisingly maintain consistent, predictable low urinary cystine concentrations, effectively reducing or preventing formation of cystine crystals that otherwise serve as seeds for stones.

As discussed above, in the context of treating cystinuria and related disorders, the modified-release compositions and systems comprise a modified-release component that releases the drug in repeated pulses over time, where a first faction is released at a first time point and a second fraction is released at a second time point, about four to about eight hours after the first time point, an interval that surprisingly gives higher bucillamine recovery in the urine, compared to longer or shorter intervals. With regard to compositions comprising bucillamine, this release strategy surprisingly leads to increased amounts of the drug in the urine, as well as increased urinary excretion of the drug, compared to immediate-release (or delayed-release) formulations. Higher, and more consistently high, bucillamine urinary concentrations result in improved therapeutic efficacy, by more consistently and effectively controlling urinary cystine concentration, as well as affording reduced side effects. In preferred embodiments, the compositions or delivery systems comprise a gastro-retentive feature to retain the therapeutic in the stomach, prolonging gastric residence time, as described herein. With regard to compositions comprising bucillamine, stomach uptake, of smaller dose amounts at a time, at critically-spaced intervals, also surprisingly leads to increased amounts of the drug in the urine, as well as increased urinary excretion of the drug, compared to immediate-release (or delayed-release) formulations.

In some embodiments, the compositions comprise a coating that begins to disintegrate or dissolve after passage of the composition through the stomach, and continues to disintegrate or dissolve as it travels along the small intestine, moving along the duodenum, followed by the jejunum, and eventually the ileum. The continued disintegration or dissolution of the coating releases the therapeutic, e.g., bucillamine, in repeated small amounts, preferably in critically-spaced pulses over time, as described herein. In a particular example, the coating releases a first amount of the therapeutic, e.g., bucillamine, when the composition passes the stomach. In some embodiments, the coating releases a second amount of the therapeutic, e.g., bucillamine, as the composition travels along the duodenum. In some embodiments, the coating releases a third amount of the therapeutic, e.g., bucillamine, as the composition travels along the jejunum. In some embodiments, the coating releases a fourth amount of the therapeutic, e.g., bucillamine, as the composition travels along the ileum. In this simple example, it can be seen that the total amount of therapeutic, e.g., bucillamine, is released as fractions of the total amount present in the composition, at different time points, throughout the passage of the composition through the small intestine.

It will be understood by the skilled artisan, based on the above disclosures, that modified-release approaches of the present invention also provide a strategy for improving efficacy and reducing side effects in other disorders, where low peak plasma levels, high urine levels, and/or high urine excretion of the drug are desirable, as well as in other disorders where immediate-release (non-coated) bucillamine formulations have been used. A disorder sharing pharmacokinetic characteristics of cystinuria, as described herein, and benefitting from similar extended-release approaches, is referred to as a “cystinuria-related disorder,” “disorder relating to cystinuria,” or a “disorder relating thereto,” e.g., where high urine excretion and/or low peak blood levels of a therapeutic agent result in improved efficacy and/or reduced toxicity. Examples of cystinuria-related disorders are other metabolic disorders of the liver and/or kidneys, where at least one physiological symptom of the disorder is reduced, delayed, or ameliorated by a thiol-binding chelating agent, such as bucillamine, and/or where thiol reduction is beneficial. Other examples of cystinuria-related disorders may include disorders where is it desirable to remove a metabolite or other substance, such as a different amino acid, present in the blood through urinary excretion to reduce or correct excess accumulation of the metabolite or substance in the urine.

Gout and rheumatoid arthritis, as well as ischemia/reperfusion injury, are examples of other conditions where immediate-release (non-coated) bucillamine formulations have been used. Rheumatoid arthritis is an autoimmune, chronic inflammatory condition that typically affects the lining of joints, causing painful swelling that can eventually result in bone erosion and joint deformity. In some cases, a wide variety of body systems also are damaged by the inflammation, including the skin, eyes, lungs, heart, and blood vessels. For rheumatoid arthritis, bucillamine is considered a disease-modifying antirheumatic drug (DMARD), that is, a drug that works to slow disease activity, mainly to slow inflammation (Sagawa et al., 2011, “A multicentre trial of bucillamine in the treatment of early rheumatoid arthritis (SNOW study),” Mod Rheumatol., 21(3): 258-259). Rheumatoid arthritis patients typically are prescribed an initial dosage schedule of 100 mg/day, that may be increased to 300 mg/day, which typically divided into three doses for administration three times a day. The goal of treatment is to stop/prevent inflammation, as well as to relieve joint pain and stiffness, prevent joint and organ damage, improve physical function and overall well-being, and/or reduce long-term complications. Nonetheless, adverse side reactions are common with prolonged bucillamine use, including skin disorders, rashes, pemphigus lesions, pruritus, gastrointestinal disturbances, interstitial pneumonitis, and kidney disorders. The major renal side effect of bucillamine for rheumatoid arthritis patients is proteinuria, caused by membranous nephropathy (Manabe, et al., 2015, “Bucillamine-Induced Membranaous Nephropathy with Crescent Formation in a patient with Rheumatoid Arthritis: Case Report and Literature Review,” Case Reports in Nephrology and Dialysis, 5:30-38). Other renal side effects include nephrotic syndrome, glomerulonephritis, and membranous nephropathy with crescent formation. Bucillamine also has been used in treatment of rheumatoid arthritis in dogs (see, e.g., https://www.researchgate.net/publication/285710918, 1994, “Effect of bucillamine on rheumatoid arthritis in a dog”).

Gout is a type of arthritis, characterized by a sudden attack of a burning pain sensation, stiffness, and/or swelling in a joint, usually a big toe. Gout may be characterized as moderate or severe gout; or chronic or acute. Over time, repeated attacks of gout can harm joints, tendons, and other tissues. Gout is caused by impaired renal excretion of uric acid, which results in excess uric acid in the blood (hyperuricemia) and ultimately the formation of urate crystals that affect the joints. The risk of developing gout increases where serum urate concentrations exceed about 400 μmol/L, and thus treatments aim to reduce serum urate levels to about below this level, that is, below about 6 mg/dL (Chao et al, 2009, “A critical reappraisal of allopurinol dosing, safety and efficacy for hyperuricemia in gout,” Curr. Rheumatol. Rep. 11: 135-1340). It has been reported that bucillamine significantly reduces mean serum urate and significantly increases mean urinary uric acid excretion. Clinical trials with bucillamine indicated that the majority patients receiving 1,800 mg bucillamine over 7 days of treatment experienced at least 50% reduction in target joint pain scores from baseline at 72 hours post-dose, without using a rescue drug (see, e.g., U.S. 2016/0000738, Example 2). The target joint pain score is an 11-point Pain Intensity Numeric Rating Scale (PI-NRS), used to assess joint pain intensity while the patient is experiencing a gout flare, rated on a scale from 0 (no pain) to 10 (worst possible pain).

The present compositions, methods, and systems, in some embodiments, find use in achieving a therapeutic effect in one or more other inflammatory conditions, including but not limited to, inflammatory intestinal disease (see, e.g., expired U.S. Pat. No. 6,025,393).

For ischemia/reperfusion (I/R) injury, bucillamine may protect against I/R injury in organ transplants. It has been reported that bucillamine significantly enhances survival of rats subjected to liver transplants, protecting against hepatic injury (Amersi, et al., 2002, “Bucillamine, a thiol antioxidant, prevents transplantation-associated reperfusion injury,” PNAS, 99(13):8915-8920). Bucillamine has been described as protecting against damage from reactive oxygen species (see, e.g., expired U.S. Pat. Nos. 5,670,545 and 5,756,547); and UV-induced skin damage (see, e.g., U.S. 2009/0169497), as well as protecting against ventricular remodeling (see, e.g., WO 2010/014953). Bucillamine also has been reported as useful in increasing lipid secretion, e.g., lipid secretion from meibomian glands of the eyes (see, e.g., U.S. 2017/0087179). The methods, compositions, and delivery systems described herein may be used to improve pharmacokinetics of bucillamine in treating or preventing one of more of these disorders or conditions, e.g., to improve efficacy and/or reduce side effects. In some embodiments, the invention provides methods of administering bucillamine to achieve an extended duration therapeutic effect in a subject with one of more of the following conditions: cystinuria, a disorder relating thereto, or a symptom thereof, rheumatoid arthritis and/or gout, a condition relating to either, or a symptom of either; and ischemia/reperfusion (I/R) injury, a disorder relating thereto, or a symptom thereof. Methods of treating a subject having one or more of these disorders or conditions, with a modified-release composition of the present invention, generally comprise a treatment course having multiple treatment periods of time with prolonged duration of effect between administrations, the treatment course comprising administering an initial treatment dose to the subject in need thereof to achieve a therapeutic effect following the initial treatment with the composition; wherein the initial treatment dose of the composition administered to the individual provides a therapeutic duration of effect, as described herein; and administering a subsequent treatment dose of the composition to the individual during or after the period of extended duration of effect, preferably according to a schedule of administration that optimizes pharmacokinetics of bucillamine for treating or preventing a given condition or symptom thereof, as described herein, e.g., so as to maintain the duration of effect.

The mode of administration used may be enteral or parenteral; or may be oral, topical, transdermal, intradermal, intramuscular, intraperitoneal, intra-articular, transmucosal, sublingual, buccal, rectal, intravaginal, intranasal, subcutaneous, intravenous, by inhalation, and the like, or any combination thereof. Preferably, oral administration is used; in some embodiments, topical administration is used, such as by applying the composition or system to the skin, buccal mucosa, or other mucosal surface.

In some embodiments, the invention provides methods of administering bucillamine to achieve an extended duration therapeutic effect in a subject with cystinuria, a disorder relating thereto, or a symptom thereof. The methods generally comprise administering to said subject a composition comprising a modified-release component and bucillamine in a first treatment dose, where fractions are released in repeated, critically-spaced pulses to provide a total daily dose of 1,200 mg or less (or an average total daily dose of 1,200 mg or less), such as about 1,100 mg or less, about 1,000 mg or less, about 900 mg or less, about 800 mg or less, about 700 mg or less, about 600 mg or less, about 500 mg or less, or about 400 mg, as the total daily dose (or average total daily dose), and/or as low as about 900 mg, about 800 mg, about 700 mg, about 600 mg, about 500 mg, or about 400 mg; and where the first treatment dose achieves the extended duration therapeutic effect having at least about an 8-hour duration of effect, such as at least about 10 hours, at least about 12 hours, at least about 14, hours, at least about 18, hours, at least about 20 hours, or about 24 hours duration of effect, or longer, preferably before a second or subsequent treatment dose is administered. In some embodiments, the subject is administered a composition comprising a modified-release component and bucillamine in a first treatment dose, where fractions are released in repeated, critically-spaced pulses to provide a total daily dose of 14 mg/kg/day or less (or an average total daily dose of 14 mg/kg/day or less), such as about 13 mg/kg/day or less, about 12 mg/kg/day, about 11 mg/kg/day or less, about 10 mg/kg/day or less, about 9 mg/kg/day or less, about 8 mg/kg/day or less, about 7 mg/kg/day or less, about 6 mg/kg/day or less, about 5 mg/kg/day or less, or about 4 mg/kg/day or less, as the total daily dose (or average total daily dose), and/or as low as about 11 mg/kg/day, about 10 mg/kg/day, about 9 mg/kg/day, about 8 mg/kg/day, about 7 mg/kg/day, about 6 mg/kg/day, about 5 mg/kg/day, or about 4 mg/kg/day; and where the first treatment dose achieves the extended duration therapeutic effect having at least about an 8-hour duration of effect, such as at least about 10 hours, at least about 12 hours, at least about 14, hours, at least about 18, hours, at least about 20 hours, or about 24 hours duration of effect, or longer, preferably before a second or subsequent treatment dose is administered. Generally, the therapeutic effect lasts up to about 10 hours, up to about 12 hours, up to about 14 hours, up to about 16 hours, up to about 18 hours, up to about 20 hours, up to about 22 hours, up to about 24 hours, up to about 30 hours, up to about 36 hours, up to about 40 hours, up to about 48 hours, up to about 72 hours, or up to about 96 hours. Doses may vary for individual patients, e.g., in extreme cases of cystinuria, the total daily dose, or average total daily dose may be about 18, mg/kg, about 20 mg/kg, about 30 mg/kg, about 35 mg/kg, or about 40 mg/kg. In some embodiments, the subject is a human patient.

In some embodiments, the subject is a dog with cystinuria, and the invention provides methods of administering bucillamine to achieve an extended duration therapeutic effect in the dog. The methods generally comprise administering a composition comprising a modified-release component and bucillamine in a first treatment dose, where fractions are released in repeated, critically-spaced pulses to provide a total daily dose of about 40 mg/kg or less (or an average total daily dose of about 40 mg/kg or less), such as about 38 mg/kg or less, about 35 mg/kg or less, about 33 mg/kg or less, about 30 mg/kg or less, about 28 mg/kg or less, about 25 mg/kg or less, about 23 mg/kg or less, or about 20 mg/kg, as the total daily dose (or average total daily dose), and/or as low as about 28 mg/kg, about 25 mg/kg, about 23 mg/kg, about 20 mg/kg, about 18 mg/kg, or about 15 mg/kg and where the first treatment dose achieves the extended duration therapeutic effect having at least about an 8-hour duration of effect, such as at least about 10 hours, at least about 12 hours, at least about 14, hours, at least about 18, hours, at least about 20 hours, or about 24 hours duration of effect, or longer, preferably before a second or subsequent treatment dose is administered. Generally, the therapeutic effect lasts up to about 10 hours, up to about 12 hours, up to about 14 hours, up to about 16 hours, up to about 18 hours, up to about 20 hours, up to about 22 hours, up to about 24 hours, up to about 30 hours, up to about 36 hours, up to about 40 hours, up to about 48 hours, up to about 72 hours, or up to about 96 hours. It will be understood that methods, compositions, systems, and kits, described herein, find use in treating dogs with cystinuria, using the doses described above.

Generally, the therapeutic effect is a reduction in the cystine concentration in the urine or kidneys, compared to that before treatment. In particular embodiments, the therapeutic effect is a urinary cystine concentration below about 250 mg/L; or a urinary cystine concentration effectively below about 250 mg/L. Achieving urinary cystine concentration “effectively below” a certain threshold means maintaining the urinary cystine concentration almost continuously below this threshold, even if there are intermittent and/or brief peaks above the threshold where such peaks are generally not high and/or not long enough to result in stone formation to the same extent, at a given time post-treatment, as if an immediate-release bucillamine formulation were used. In some embodiments, the therapeutic effect is a urinary bucillamine recovery above about 25%, above about 30%, or above about 35% of the total dose administered, preferably above about 40%, above about 45%, or above about 50%, and more preferably above about 55%, above about 60%, above about 65%, or still more preferably above about 70% of the total dose administered. For example, total bucillamine urinary recovery may be about 25% to about 75%, about 30% to about 70%, about 30% to about 60%, about 40% to about 60%, or about 50% of the total dose administered, collected over the extended period of therapeutic effect, e.g., about 12 hours.

In preferred embodiments, following administration of a first treatment dose of the modified-release composition or delivery system, the therapeutic effect lasts for several hours. For example, the therapeutic effect may last for at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 22 hours, or about 24 hours or longer. In a particular embodiment, the modified-release formulation provides about 12 hours of controlled release, providing repeated, critically-spaced release of smaller amounts of the total bucillamine, at specified time points, during the extended period. Accordingly, in such preferred embodiments, a single treatment affords longer duration, thus allowing a dosing schedule with longer periods between administrations, and fewer doses per day that require action on the part of the subject.

In particularly preferred embodiments, an evening dose of the modified-release composition or delivery system provides therapeutic effects through all or most of the night, typically when the patient sleeps, maintaining higher, and consistently higher, levels of bucillamine in urine, which in turn maintains lower and, importantly, consistently lower, cystine levels in the urine, when compared to non-coated bucillamine (such as Rimatil®). As discussed above, where the therapeutic diminishes significantly hours after administration, the patient is left unprotected for most of the night when urinary cystine levels rise and the seeds of cystine stones form. Modified-release compositions and systems of the invention, in preferred embodiments, thus provide better control of cystine stone formation, resulting in fewer and/or less frequent and/or smaller cystine stones compared to those experienced by cystinuria patients taking a non-coated bucillamine. For example, patients forming at least one stone per week, while taking non-coated bucillamine, may experience a reduction to no more than one stone per month, one stone in six months, one stone per year, one stone in five years, or no more than one stone in ten years, using a modified-release composition or system of the present invention. As another example, patients forming several stones per week, while taking non-coated bucillamine, may experience a reduction to no more than 2-3 stones a week, one stone a week, one stone per month, one stone a year, or no more than one stone in five years, using a modified-release composition or system of the present invention. In some embodiments, the modified-release composition reduce one or more other symptoms of cystinuria or a disorder related thereto as the therapeutic effect, e.g., reducing frequency and/or severity of, or preventing blood in the urine, severe pain in the side or the back, nausea, vomiting, and/or pain near the groin, pelvis, or abdomen.

A dosing schedule, as described herein, refers to frequency of administering or applying the pharmaceutical composition or delivery system, and generally requires some action, or conscious action, on the part of the subject. Actions may include, for example, swallowing a modified-release tablet, applying a modified-release skin patch to an appropriate part of the body, or placing a modified-release wafer to the inside of the cheek. Administration or application requiring such conscious action generally is distinguished from release of the drug that occurs following such administration/application, such as the repeated, critically-spaced pulses released by the modified-release components described herein (and which do not require a conscious action on the part of the subject).

In preferred embodiments, in the context of treating cystinuria or related disorder, the modified-release composition or system is administered or applied no more than twice a day. Generally, twice a day administration or application will involve dosing once in the morning, e.g., to protect against high urinary cystine during the day; and once in the evening, e.g., to protect against high urinary cystine during the night. In a particular embodiment, the modified-release composition or system is administered or applied twice a day. In more preferred embodiments, the modified-release composition or system is administered or applied once a day. Thus, the present delivery methods achieve less frequent dosing that requires any conscious action on the part to the subject, such as dosing 1 or 2 times/day versus 3 or more times/day, as in the case of current bucillamine study designs. In other embodiments, the modified-release composition or system is administered or applied three or four times a day, preferably less than four times a day, but at lower total and/or lower average total daily doses, compared to what would be required using uncoated bucillamine.

In preferred embodiments, modified-release components described herein, including gastro-retentive features, are used to improve pharmacokinetics of bucillamine in treating cystinuria, or a related disorder, preferably reducing administrations to twice a day, more preferably once a day. In a particularly preferred embodiment, once a day administration of a composition of the invention achieves four pulses of drug in the stomach, at critically-spaced intervals according to aspects of this invention. In some embodiments, once a day administration of a composition of the invention achieves three pulses of drug in the stomach and one in the duodenum, at critically-spaced intervals. In some embodiments, twice a day administration of a composition of the invention achieves four pulses of drug in the stomach, at critically-spaced intervals. In some embodiments, twice a day administration of a composition of the invention achieves two pulses of drug in the stomach and two in the duodenum, at critically-spaced intervals. In some embodiments, once a day administration of a composition of the invention achieves two pulses of drug in the stomach, one in the duodenum, and one in the ileum or jejunum, at critically-spaced intervals. In some embodiments, once a day administration of a composition of the invention achieves one pulse of drug in the stomach, one in the duodenum, one in ileum or jejunum, and one in the large intestine, at critically-spaced intervals according to aspects of this invention.

In some embodiments, the invention provides methods of administering bucillamine to achieve an extended duration therapeutic effect in a subject with rheumatoid arthritis, a condition relating thereto, or a symptom thereof. A “rheumatoid arthritis-related condition,” “condition relating to rheumatoid arthritis,” or a “condition relating thereto” encompasses a condition that shares common etiology, progress of disease, and/or symptoms as rheumatoid arthritis and that can be treated similarly with bucillamine. A rheumatoid arthritis-related condition may include, e.g., other autoimmune conditions, other chronic inflammatory conditions, and/other conditions damaging the lining of the joints. Methods for treating rheumatoid arthritis, a condition relating thereto, or a symptom thereof, generally comprise administering orally to said subject a composition comprising a modified-release bucillamine in a first treatment dose, where dosing is repeated to provide a total daily dose of 300 mg or less (or an average total daily dose of 300 mg or less), such as about 200 mg or less, about 150 mg or less, about 100 mg or less, about 80 mg or less, or about 50 mg, as the total daily dose (or average total daily dose), and/or as low as about 150 mg/day, about 100 mg/day, about 80 mg/day, about 60 mg/day, about 50 mg/day, or abut 30 mg/day; and where the first treatment dose achieves the extended duration therapeutic effect having at least about an 8-hour duration of effect, such as at least about 10 hours, at least about 12 hours, at least about 14, hours, at least about 18, hours, at least about 20 hours, or about 24 hours duration of effect, preferably before a second or subsequent treatment dose is administered. Generally, the therapeutic effect in the context of treating or preventing rheumatoid arthritis, or a related condition, may last up to about 10 hours, up to about 12 hours, up to about 14, hours, up to about 18, hours, up to about 20 hours, up to about 24 hours, up to about 36 hours, up to about 48 hours, up to about 72 hours, or up to about 96 hours. Doses may vary for individual patients, e.g., with severe rheumatoid arthritis, the total daily dose, or average total daily dose may be about 150 mg, about 200 mg, about 300 mg, about 400 mg, or about 500 mg.

In preferred embodiments, modified-release components described herein, including gastro-retentive features, are used to improve pharmacokinetics of bucillamine in treating and or preventing rheumatoid arthritis, or a related condition, preferably reducing administrations to twice a day, more preferably once a day. In a particularly preferred embodiment, once a day administration of a composition of the invention achieves four pulses of drug in the stomach, at critically-spaced intervals according to aspects of this invention. In some embodiments, once a day administration of a composition of the invention achieves three pulses of drug in the stomach and one in the duodenum, at critically-spaced intervals. In some embodiments, twice a day administration of a composition of the invention achieves four pulses of drug in the stomach, at critically-spaced intervals. In some embodiments, twice a day administration of a composition of the invention achieves two pulses of drug in the stomach and two in the duodenum, at critically-spaced intervals. In some embodiments, once a day administration of a composition of the invention achieves two pulses of drug in the stomach, one in the duodenum, and one in the ileum or jejunum, at critically-spaced intervals. In some embodiments, once a day administration of a composition of the invention achieves one pulse of drug in the stomach, one in the duodenum, one in ileum or jejunum, and one in the large intestine, at critically-spaced intervals according to aspects of this invention.

Generally, the therapeutic effect in the context of rheumatoid arthritis is one or more of reduced inflammation, relief of joint pain and/or stiffness, reduced joint and organ damage, improved physical function and overall well-being, and/or reduced long-term complications. Therapeutic effect may also be prevention of one or more symptoms of rheumatoid arthritis in a patient identified as likely to develop the condition, e.g., based on medical history and/or early indicators of disease propensity. In preferred embodiments, use of lower and/or less frequent bucillamine administrations results in reduced adverse side effects typically associated with bucillamine treatment of rheumatoid arthritis, such as a reduction in frequency, duration, and/or severity of one or more of skin disorders, rashes, pemphigus lesions, pruritus, interstitial pneumonitis, gastrointestinal disturbances, and kidney disorders, including kidney disorders such as nephrotic syndrome, glomerulonephritis, membranous nephropathy, and membranous nephropathy with crescent formation.

In some embodiments, the invention provides methods of administering bucillamine to achieve an extended duration therapeutic effect in a subject with gout, a condition relating thereto, or a symptom thereof. A “gout-related condition,” “condition relating to gout,” or a “condition relating thereto” encompasses a condition that shares common etiology, progress of disease, and/or symptoms as gout and that can be treated similarly with bucillamine. A gout-related condition may include, e.g., other conditions caused by impaired renal excretion of uric acid, other conditions characterized by sudden attacks of joint pain, or other conditions where repeated attacks cause harm to joints and tendons. A “gout-related condition” also may refer to a metabolic disorder characterized by a cluster of one, two, three, four, or all of the following conditions: abdominal (central) obesity, elevated blood pressure, elevated fasting plasma glucose, high serum triglycerides, and low high-density lipoprotein levels. See, e.g., U.S. 2016/0000738.

Methods for treating or preventing gout, a condition relating thereto, or a symptom thereof, generally comprise administering orally to said subject a composition comprising a modified-release bucillamine in a first treatment dose, where dosing is repeated to provide a total daily dose of 250 mg or less (or an average total daily dose of 250 mg or less), such as about 200 mg or less, about 150 mg or less, about 120 mg or less, about 100 mg or less, about 75 mg or less, or about 50 mg, as the total daily dose or average total daily dose, and/or as low as about 120 mg/day, about 100 mg/day, about 75 mg/day, about 50 mg/day, about 30 mg/day, or about 25 mg/day; and where the first treatment dose achieves the extended duration therapeutic effect having at least about an 8-hour duration of effect, such as at least about 10 hours, at least about 12 hours, at least about 14 hours, at least about 18 hours, at least about 20 hours, or about 24 hours duration of effect, preferably before a second or subsequent treatment dose is administered. Generally, the therapeutic effect in the context of treating or preventing gout, or a related condition, may last up to about 10 hours, up to about 12 hours, up to about 14 hours, up to about 18 hours, up to about 20 hours, up to about 24 hours, up to about 36 hours, up to about 48 hours, or up to about 72 hours, or up to about 96 hours. In some even more preferred embodiments, the modified-release composition is administered no more than every other day, such as, e.g., every other day, or twice a week, still even more preferably once a week.

Generally, the therapeutic effect in the context of treating gout or related condition, is a reduction in target joint pain scores from baseline at 72 hours post-dose, without using a rescue drug. In particular embodiments, the therapeutic effect is at least a 50% reduction in target joint pain scores from baseline, without using a rescue drug, preferably at least about a 60%, more preferably at least about a 70%, or still more preferably at least about a 75% reduction in target joint pain scores. Therapeutic effect may also be prevention of one or more symptoms of gout in a patient identified as likely to develop the disorder, e.g., based on medical history and/or early indicators of disease propensity. For example, the therapeutic effect may be reduced frequency of gout attacks, or reduced average length of individual attacks, and/or reduced average severity of individual attacks. In some embodiments, the gout to be treated is acute gout, such as an acute gout flare. In some embodiments, the gout to be treated is chronic gout and, in such cases, drug may be administered chronically to prevent gout attacks

Urate levels also provide a measure of the therapeutic effect in treating gout, such as a reduction in the urate concentration in the blood or urine compared to that before treatment. In particular embodiments, the therapeutic effect is a serum urate concentration below or effectively below about 6 mg/dL (that is, below or effectively below about 357 μmol/L). Achieving serum urate concentration “effectively below” a certain threshold means maintaining the serum urate concentration almost continuously below this threshold, even if there are intermittent and/or brief peaks above the threshold where such peaks are generally not high and/or not long enough to result in symptoms of gout to the same extent, at a given time post-treatment, as if an immediate-release bucillamine formulation were used.

In some embodiments, the invention provides methods of administering bucillamine to achieve an extended duration therapeutic effect in a subject with or at risk for ischemia/reperfusion injury, a disorder relating thereto, or a symptom thereof. Such a subject may be a patient about to have, having, or who has had an organ transplant, such a, liver transplant. The methods generally comprise administering orally to said subject a composition comprising a modified-release bucillamine in a first treatment dose, where the first treatment dose achieves the extended duration therapeutic effect, preferably before a second or subsequent treatment dose is administered, when compared to administration of immediate-release bucillamine. Generally, the therapeutic effect is protection against hepatic injury, which can be measured in terms of improved portal vein blood flow, increased bile production, preservation of normal liver architecture, reduced liver enzyme release, reduced indices of oxidative stress, reduced levels of glutathione in the liver, reduced levels of oxidized glutathione levels in the liver and/or blood, and the like, or any combination thereof.

Modified-release compositions or systems of the invention are administered or applied so as to deliver an effective amount of a therapeutic agent, e.g., a therapeutically effective amount of bucillamine. A “therapeutically effective amount” or an “effective amount” of bucillamine refers to a dose of bucillamine that brings about at least one therapeutic, desired, or positive benefit, such as reducing, alleviating, decreasing, diminishing, ameliorating, or curing at least one symptom of cystinuria or a disorder related thereto in a subject in need thereof; or reducing, alleviating, decreasing, diminishing, ameliorating, or curing at least one symptom of rheumatoid arthritis or gout, or a condition related to either, in a subject in need thereof, or reducing, alleviating, decreasing, diminishing, ameliorating, or curing at least one symptom of a condition that previously has been treated with bucillamine in a subject in need thereof.

The term therapeutically effective amount also implies a safe amount, i.e. one low enough to avoid serious adverse effects or to avoid adverse effects that are not outweighed by therapeutic benefit achieved for a particular patient or group of patients. The modified-release formulations of the invention generally contain an appropriately effective amount of bucillamine to provide a treatment dose in a single administration/application of a dosage form. Alternatively, the modified release compositions or systems can provide, e.g., a half, a third, or a quarter of the single treatment dose, so that 2, 3, or 4 tablets or capsules are taken at a given time.

Exact effective amounts may vary from subject to subject, depending on the disorder being treated, the severity of the disorder, age, weight, and general condition of the subject, treatment history, and the like. An important factor determining the specific bucillamine dose for treating cystinuria is the untreated (baseline) urinary cystine concentrations in a given patient. For example, consider patient A, having an untreated urinary cystine concentration of 400 mg/L and needing X mg/L of bucillamine in the urine to keep cystine levels at 250 mg/L or less or effectively below that value. Patient B, having untreated urinary cystine concentrations of 1,200 mg/L would need 3X mg/L of bucillamine in urine to keep cystine levels at 250 mg/L or less, or effectively below that value. A suitable effective amount for an individual may be determined by techniques known in the art and/or described herein, e.g., measuring urinary cystine concentrations, such as measuring urinary cystine concentrations over a 24 hour period and using the urinary concentration of free cystine to adjust the dose. One approach calculates total urinary excretion of cystine as the sum of free cystine and the amount of cystine of the bucillamine-cysteine mixed disulfide. Another method for measuring urinary cystine involves “cystine capacity” (https://www.ncbi.nlm.nih.gov/pubmed/15865542). Other approaches measure urinary bucillamine using HPLC based on post-column reaction with dithionitrobenzene (DTNB). Blood levels of bucillamine also can be measured, e.g., using specific and sensitive gas chromatography-mass spectrometry (Lee et al., 2009, “Development and validation of a reversed-phase fluorescence HPLC method for determination of bucillamine in human plasma using pre-column derivatization with monobromobimane,” Journal of Chromatography B, 877: 2130-2134). Doses also may be considered in terms of mg bucillamine/kg or mg bucillamine/m², considering respectively the body weight or body surface area of the patient. Similar dosing considerations, as described above, apply to dogs or other mammals suffering from cystinuria.

In preferred embodiments, use of the modified-release composition or system improves efficacy and reduces the total daily dose of bucillamine compared to the average daily dose expected if using currently-available bucillamine (Rimatil®). That is, the present approaches may achieve efficacy using a total daily dose lower than 800 mg bucillamine/day, or lower than about 10 mg/kg/day. In more preferred embodiments, use of the modified-release composition or system reduces toxicity and decreases severity and/or incidence of adverse side effects compared to those typically experienced by patients taking Rimatil®.

Lower peak blood levels of bucillamine result in fewer side effects, generally seen with use of bucillamine and/or other cystinuria drugs. In preferred embodiments, the methods, compositions, and/or delivery systems described herein result in fewer of at least one of the following side effects: rash, itch, proteinuria (dark urine), canker sores/abnormal feeling inside mouth; erythema, respiratory distress, dizziness, anaphylactoid symptoms, fever, sore throat, bleeding tendency, agranulocytosis, thrombocytopenia, interstitial pneumonitis, pulmonary fibrosis, decreased urination output, swelling, acute renal failure, nephrotic syndrome, loss of appetite, fatigue, yellowing of the skin or eyes, liver function disorder, jaundice; as well as in some cases, gastrointestinal side-effects (anorexia, abdominal pain, bloating, diarrhea, emesis, flatus, nausea, soft stools); taste/smell impairment; dermatologic complications (ecchymosis, elastosis perforans serpiginosa, oral ulcers, pemphigus, pharyngitis, prurites, rash, urticaria, warts, wrinkling); hypersensitivity reactions (adenopathy, arthralgia, chills, dyspnea, fatigue, fever, laryngeal edema, myalgia, respiratory distress, weakness); hematologic abnormalities (anemia, eosinophilia, increased bleeding, leukopenia, thrombocytopenia); renal complications (hematuria, nephrotic syndrome, proteinuria); pulmonary manifestations (bronchiolitis, dyspnea, hemoptysis, pulmonary infiltrates); neurologic complications (myasthenic syndrome); pain, such as renal colic, urinary urgency, sore throat, bleeding, easy bruisability, agranulocytosis, Goodpasture's syndrome, myasthenia gravis, proteinuria, nephrotic syndrome, and membranous glomerulopathy. A reduction in side effects can refer to less of a particular side effect (e.g., fewer occurrences or no occurrences of dizzy episodes for a given patient over a given time frame); to less severe manifestations of a particular side effect (e.g., less severe dizziness for a given patient for each, some, or most occurrences of dizziness); or a combination thereof. In more preferred embodiments, the side effects are sufficiently mild and/or infrequent to allow patients to continue treatment, or to allow a greater percentage of patients to continue treatment than seen with other cystinuria treatments, such as Thiola®. In most preferred embodiments, the side effects are sufficiently mild and/or infrequent to allow at least about 50 to about 60%, at least about 60 to about 70%, at least about 70 to about 80%, or at least about 80 to about 90%, to continue treatment for at least about a year, at least about 5 to about 10 years, at least about 5 to about 15 years, at least about 10 to about 15 years, at least about 10 to about 20 years, or for the remainder of the patient's life.

As discussed above, repeated, critically-spaced release of smaller doses, over an extended period of time, decreases peak blood levels and increases urinary concentrations and urine excretion of the drug, as well as altering its distribution, preferably to statistically significant extents and/or surprising extents compared to immediate-release (or delayed-release) versions. Decreased blood levels and distribution in turn reduce adverse side effects of the drug. Also as discussed above, pulsed delivery, particularly in the stomach, according to the invention improves absorption and bioavailability compared to use of only immediate-release formulations that generally are released in the small intestines as well as the stomach. Moreover, increased urinary excretion provides higher concentrations of bucillamine in urine, where it acts to reduce cystine levels in cystinuria patients, allowing for use of a lower total daily dose. The present invention provides a surprising critical range for interval lengths between consecutive pulses of the drug, that unexpectedly maintains higher and more consistently higher urinary bucillamine concentrations, compared with intervals longer or shorter than those within a critical range. The modified-release strategies of the present invention thus allow use of lower doses, surprisingly increasing efficacy of bucillamine. Lower doses further reduce adverse side effects, as well as improving patient compliance and quality of life.

In particular embodiments, the total daily dose used for cystinuria patients is less than about 800 mg/day or less than about 10 mg/kg/day. For example, one dosing schedule comprises treatment doses of about 400 mg each, administered twice a day, or less than about 5 mg/kg/day. In another example, the dosing schedule comprises doses of about 300 mg each, or about 3 mg/kg each, administered or applied twice a day; in another example, the dosing schedule comprises doses of about 200 mg each, or about 2 mg/kg each, administered or applied twice a day. In a preferred embodiment, the dosing schedule comprises a single administration or application a day of a treatment dose of about 800 mg, about 700 mg, about 600 mg, about 500 mg, or about 400 mg, or of about 10 mg/kg/day, about 7.5 mg/kg/day, about 6.5 mg/kg/day, or about 5 mg/kg/day. In various embodiments, the total daily dose may range from about 400 mg to about 800 mg, with individual doses ranging from about 200 mg to about 400 mg, for twice daily dosing, or from about 400 mg to about 800 mg for once daily dosing. In other embodiments, the total daily dose may range from about 800 mg to about 1,200 mg, with individual doses ranging from about 400 mg to about 600 mg, for twice daily dosing, or from about 800 mg to about 1,200 mg for once daily dosing. In various embodiments, the total daily dose may range from about 5 mg/kg/day to about 10 mg/kg/day, with individual doses ranging from about 2.5 mg/kg to about 5 mg/kg for twice daily dosing, or from about 5 mg/kg to about 10 mg/kg for once daily dosing. In some embodiments, the total daily dose may range from about 10 mg/kg/day to about 12 mg/kg/day, with individual doses ranging from about 5 mg/kg to about 6 mg/kg for twice daily dosing, or from about 10 mg/kg to about 12 mg/kg for once daily dosing.

Oral dosage forms generally comprise a tablet or capsule of a modified-release bucillamine providing about 100 mg-1,000 mg bucillamine. Depending on the unit dosage of a given dosage form, the appropriate number of tablets can be taken to provide effective doses according to the dosing schedule being followed. In some embodiments, the delivery methods of the present invention allow lower doses compared to those required when using an immediate-release formula. For example, the daily dose, or average daily dose, for a given patient may be reduced by at least about 80%, at least about 70%, at least about 60%, at least about 50%, at least about 40%, at least about 30%, or at least about 20% of the dose administered when using an immediate-release formula. In some embodiments, the daily dose, or average daily dose, for a given patient is reduced by about 80%, about 70%, about 60%, about 50%, about 40%, about 30%, or about 20% of the dose administered when using an immediate-release formula.

In some embodiments, the delivery methods of the present invention achieve lower peak blood levels of total bucillamine compared to immediate-release (or delayed-release) formulations. For example, where a dose of 30 mg/kg is administered orally over a twelve hour period, modified-release strategies described herein can achieve peak blood levels of total bucillamine of no more than about 30,000 ng/mL or no more than about 25,000 ng/mL, preferably no more than about 23,000 ng/mL or no more than about 22,000 ng/mL, more preferably no more than about 20,000 ng/mL or no more than about 18,000 ng/mL, and still more preferably no more than about 17,000 ng/mL or no more than about 15,000 ng/mL. In some embodiments, following oral administration of a formulation of the invention and/or during an extended-release dosage regimen, peak blood total bucillamine levels are lower than about half that obtained following oral administration of immediate-release formulations and/or following immediate-release dosage regimens, preferably lower than about a third, more preferably lower than about a quarter, of that obtained using immediate-release formulations and/or immediate-release dosage regimens. In some embodiments, where a dose of 30 mg/kg is administered orally over a twelve hour period, according to modified-release strategies described herein, peak blood levels of total bucillamine may reach no more than about 25,000 to about 30,000 ng/mL, about 22,000 to about 27,000 ng/mL, about 20,000 to about 25,000 ng/mL, about 17,000 to about 22,000 ng/mL, about 15,000 to about 20,000 ng/mL, about 12,000 to about 17,000 ng/mL, or about 10,000 to about 15,000 ng/mL total bucillamine.

In some embodiments, the delivery methods achieve lower, and more consistently lower, free bucillamine plasma concentrations compared to immediate-release (or delayed-release) formulations. For example, where a dose of 30 mg/kg is administered orally over a twelve hour period, modified-release strategies described herein can achieve free (unbound) bucillamine peak blood levels of less than about 4,000 ng/mL, less than about 3,500 ng/mL, less than about 3,000 ng/mL, less than about 2,500 ng/mL, or less than about 2,000 ng/mL. Preferably, in some embodiments where a dose of 30 mg/kg is administered orally over a twelve hour period, modified-release strategies described herein achieve a free bucillamine peak blood level of a value between about 2,500 and about 3,000 ng/mL; between about 2,000 and about 2,500 ng/mL; between about 1,700 and about 2,200 ng/mL or between about 1,500 and about 2,000 ng/mL free bucillamine. In some embodiments, where a dose of 30 mg/kg is administered orally over a twelve hour period, according to modified-release strategies described herein, the strategies may achieve free bucillamine concentrations in the plasma between about 500 and about 2,500 ng/mL, for the at least about 8 hours, at least about 10 hours, or preferably for at least about 12 hours of extended duration of effect; between about 500 and about 2,000 ng/mL, for the at least about 8 hours, at least about 10 hours, or preferably for at least about 12 hours of extended duration of effect; between about 800 and about 2,000 ng/mL, for the at least about 8 hours, at least about 10 hours, or for at least about 12 hours of extended duration of effect; between about 800 and about 1,500 ng/mL, for the at least about 8 hours, at least about 10 hours, or for at least about 12 hours of extended duration of effect; or between about 500 and about 1,500 ng/mL, for the at least about 8 hours, at least about 10 hours, or for at least about 12 hours of extended duration of effect. Generally, the lower, and more consistently lower, free bucillamine plasma concentrations are maintained up to about 10 hours, up to about 12 hours, up to about 16 hours, up to about 20 hours, up to about 22 hours, up to about 24 hours, up to about 36 hours, or up to about 48 hours.

In the context of treating or preventing inflammatory conditions, such as gout, rheumatoid arthritis, a condition relating to either, or a symptom of either, free bucillamine is an important pharmacokinetic parameter, without regard to the amount of drug recovered in urine. As discussed above, in this context, a modified-release strategy providing more closely spaced repeat pulses, particularly when released in the stomach, provides a preferred strategy. The specific bucillamine dose may be selected based on the untreated serum urate concentrations in a given patient, e.g., in the context of treating/prevention gout. In preferred embodiments for treating inflammatory conditions, like gout or rheumatoid arthritis, the present delivery methods achieve higher and steadier free bucillamine concentrations in the plasma compared to other methods. For example, in some embodiments for treating/preventing gout or rheumatoid arthritis, free bucillamine plasma levels are maintained between about 500 and about 4,000 ng/mL, for the at least about 8 hours, at least about 10 hours, or preferably for at least about 12 hours of extended duration of effect; between about 1,000 and about 4,000 ng/mL, for the at least about 8 hours, at least about 10 hours, or preferably for at least about 12 hours of extended duration of effect; between about 1,000 and about 3,500 ng/mL, for the at least about 8 hours, at least about 10 hours, or for at least about 12 hours of extended duration of effect; between about 800 and about 3,500 ng/mL, for the at least about 8 hours, at least about 10 hours, or for at least about 12 hours of extended duration of effect; between about 800 and about 3,000 ng/mL, for the at least about 8 hours, at least about 10 hours, or for at least about 12 hours of extended duration of effect, or between about 1,000 and about 3,000 ng/mL, for the at least about 8 hours, at least about 10 hours, or for at least about 12 hours of extended duration of effect. Generally, the higher, and more consistently higher, free bucillamine plasma concentrations are maintained up to about 10 hours, up to about 12 hours, up to about 16 hours, up to about 20 hours, up to about 22 hours, up to about 24 hours, up to about 36 hours, or up to about 48 hours. In some even more preferred embodiments, the higher, and more consistently higher, free bucillamine plasma concentrations are maintained, for at least about one day, at least about two days, at least about three days, at least about four days, at least about five days, at least about six days, at least about one week, at least about ten days, at least about two weeks, at least about three weeks, or at least about a month; and/or up to about 48 hours, up to about 3 days, up to about 4 days, up to about 5 days, up to about 6 days, up to about 7 days, up to about 10 days, up to about 2 weeks, up to about 3 weeks, up to about a month, up to about a month and a half, or up to about two months.

In some embodiments, the delivery methods achieve higher, and more consistently higher, urinary bucillamine concentrations compared to immediate-release formulations or delayed-release formulations, a pharmacokinetic parameter of importance in treating cystinuria or related disorders. The specific bucillamine dose may be selected based on the untreated urinary cystine concentrations in a given patient. Nonetheless, in preferred embodiments, the present delivery methods give a urinary bucillamine recovery above about 30% of the total dose administered for the at least about 8 hours, at least about 10 hours, or preferably for at least about 12 hours of extended duration of effect; more preferably above about 40% of the total dose administered for the at least about 8 hours, at least about 10 hours, or for at least about 12 hours of extended duration of effect; still more preferably above about 50% of the total dose administered for the at least about 8 hours, at least about 10 hours, or for at least about 12 hours of extended duration of effect; and yet still more preferably above about 60% of the total dose administered for the at least about 8 hours, at least about 10 hours, or for at least about 12 hours of extended duration of effect, e.g., up to about 10 hours, up to about 12 hours, up to about 16 hours, up to about 20 hours, up to about 22 hours, up to about 24 hours, up to about 36 hours, or up to about 48 hours. To illustrate, a bucillamine urinary recovery above about 50% means that, if a half-daily dose of 500 mg bucillamine is administered, in accordance with a strategy of the invention, total bucillamine recovered in the urine, over the 12 hour time period following administration, would be about 250 mg or more. In some embodiments, bucillamine urinary recovery may be up to about 40%, about 50%, about 60%, about 65%, or about 70%, of the total dose administered. Generally, the higher, and more consistently higher, urinary total bucillamine concentrations are maintained up to about 10 hours, up to about 12 hours, up to about 16 hours, up to about 20 hours, up to about 22 hours, up to about 24 hours, up to about 36 hours, or up to about 48 hours.

In some embodiments, the delivery methods achieve lower, and more consistently lower, urinary cystine concentrations compared to immediate-release (or delayed-release) formulations, such as keeping urinary cystine below, or effectively below, a “baseline level” for a particular patient, that is, below that patient's untreated urinary cystine level, down to about normal urinary cystine concentrations for the at least about 8 hours, at least about 10 hours, or preferably for at least about 12 hours of extended duration of effect. In some embodiments, the delivery methods achieve lower, and more consistently lower, urinary cystine concentrations compared to immediate-release (or delayed-release) formulations, such as keeping urinary cystine below, or effectively below, about 250 mg/L and down to about normal urinary cystine concentrations for the at least about 8 hours, at least about 10 hours, or preferably for at least about 12 hours of extended duration of effect; preferably below, or effectively below, about 200 mg/L and down to about normal urinary cystine concentrations for the at least about 8 hours, at least about 10 hours, or for at least about 12 hours of extended duration of effect; more preferably below, or effectively below, about 150 mg/L and down to about normal urinary cystine concentrations for the at least about 8 hours, at least about 10 hours, or for at least about 12 hours of extended duration of effect; and even more preferably at or below, or effectively below, about 100 mg/L and down to about normal urinary cystine concentrations for the at least about 8 hours or for at least about 12 hours of extended duration of effect. In more preferred embodiments, urinary cystine is kept effectively below about 250 mg/L, about 200 mg/L, about 150 mg/L, or about 100 mg/L, and down to about normal urinary cystine concentrations, for greater than 12 hours, such as for at least about 14 hours, at least about 16 hours, at least about 18 hours, at least about 20 hours, or at least about 24 hours, or longer. Normal cystine concentrations are about 10 to about 250 mg/L, e.g., about 20 to about 200 mg/L, or about 50 to about 150 mg/L. Generally, the lower, and more consistently lower, urinary cystine concentrations are maintained up to about 10 hours, up to about 12 hours, up to about 16 hours, up to about 20 hours, up to about 22 hours, up to about 24 hours, up to about 36 hours, or up to about 48 hours.

Extended release strategies of the present invention can be used in combination with one or more other therapies useful for treating cystinuria and related disorders. A nonlimiting example is use of agents to increase pH of urine, such as, alkali metal salts of citric acid, alkaline-earth metal salts of citric acid, potassium citrate, potassium nitrate, and/or sodium bicarbonate, in conjunction with a modified-release composition or system described herein. Additional approaches from increasing alkalinity of urine are described, e.g., in U.S. 2017/0172960 A1, to Saadeh et al., entitled “Pharmaceutical formulations for Treating Kidney Stones and Methods for Fabricating and Using Thereof.” In other embodiments, the present invention can be used in combination with one or more other therapies useful for treating rheumatoid arthritis and/or gout. For example, a patient with rheumatoid arthritis may be administered an extended-release bucillamine composition, as described herein, in combination with a CaMKII inhibitor (see, e.g., U.S. 2008/0255121) and/or tranilast (see, e.g., U.S. 2010/0158905), either in the same composition or a separate composition. As another example, a patient with gout may be administered an extended-release bucillamine composition or delivery system, as described herein, in combination with allopurinol and/or colchicine (see, e.g., WO 2014/078956), either in the same composition or a separate composition.

Methods and Uses regarding Dosing Schedules

Still another aspect of the invention provides methods and uses of pharmaceutical compositions according to a dosing schedule that provides repeated, critically-spaced pulses of a therapeutic agent, in accordance with aspects of the invention. In some embodiments, bucillamine-containing pharmaceutical compositions are administered to a subject with cystinuria or a related disorder at critically-spaced intervals that provide consistently higher bucillamine levels in the urine than longer or shorter intervals. In the simplest case, the pharmaceutical composition comprises a pharmaceutically acceptable carrier and bucillamine, preferably in an oral formulation, but not necessarily with a modified-release component nor an enteric coating. In some embodiment, the composition further comprises a gastro-retentive feature, e.g., to further promote adsorption of bucillamine in the stomach.

For example, the bucillamine can be provided in a treatment dose for repeated administration to the subject with cystinuria in intervals according to the present teachings, for example, with about four to about eight hours between consecutive administrations. Generally, the composition is administered no more frequently than every four hours, that is no more than about six times a day, so that the interval is no less than about four hours. In a preferred embodiment, the interval is about six hours and the composition is administered about four times a day. In some embodiments, administration provides a total daily dose of about 1,200 mg or less, or of about 14 mg/kg or less; or an average total daily dose of about 1,200 mg or less, or of about 14 mg/kg or less, where the dosage indicates the amount of active ingredient (bucillamine). Accordingly, the invention provides methods of administering a therapeutically effective amount of bucillamine to achieve a therapeutic effect in a subject with cystinuria, a disorder relating thereto, or a symptom thereof, by administering a pharmaceutical composition comprising bucillamine, in a treatment dose, and a pharmaceutically acceptable carrier; re-administering said pharmaceutical composition about four to about eight hours after said first administration; and repeating these administering steps to provide a total daily dose of 1,200 mg or less (or of 14 mg/kg/day or less).

In some embodiments, bucillamine-containing pharmaceutical compositions are administered to a subject with an inflammatory condition, such as gout or rheumatoid arthritis, at shorter critically-spaced intervals, that provide consistently higher free bucillamine levels in the plasma (without regard to urinary levels) than longer or shorter intervals. In the simplest case, the pharmaceutical composition comprises a pharmaceutically acceptable carrier and bucillamine, preferably in an oral formulation, but not necessarily with a modified-release component nor an enteric coating. In some embodiment, the composition further comprises a gastro-retentive feature, e.g., to further promote adsorption of bucillamine in the stomach.

For example, the bucillamine can be provided in a treatment dose for repeated administration to the subject with gout or rheumatoid arthritis in intervals according to the present teachings, for example, with about two to about six hours between consecutive administrations. Generally, the composition is administered no more frequently than every three hours, that is no more than about eight times a day, so that the interval is no less than about three hours. In a preferred embodiment, the interval is about four hours and the composition is administered about six times a day.

For treating or preventing an inflammatory condition, the pharmaceutical composition generally is delivered so as to provide a total daily dose of bucillamine used for that condition. For example, in some embodiments in the context of treating/preventing rheumatoid arthritis, a related condition or symptom thereof, administration provides a total daily dose of about 300 mg or less, or of about 3 mg/kg or less; or an average total daily dose of about 300 mg or less, or of about 3 mg/kg or less, where the dosage indicates the amount of active ingredient (bucillamine). Accordingly, the invention provides methods of administering a therapeutically effective amount of bucillamine to achieve a therapeutic effect in a subject with rheumatoid arthritis, a condition relating thereto, or a symptom thereof, by administering a pharmaceutical composition comprising bucillamine, in a treatment dose, and a pharmaceutically acceptable carrier; re-administering said pharmaceutical composition about two to about six hours after said first administration; and repeating these administering steps to provide a total daily dose of about 300 mg or less, that is, about 3 mg/kg/day or less (or an average total daily dose of about 300 mg or less, or about 3 mg/kg/day or less), such as about 200 mg or less, about 150 mg or less, about 100 mg or less, about 80 mg or less, or about 50 mg, as the total daily dose (or average total daily dose), and/or as low as about 150 mg/day, about 100 mg/day, about 80 mg/day, about 60 mg/day, about 50 mg/day, or abut 30 mg/day.

In some embodiments, in the context of treating/preventing gout, a related condition or symptom thereof, administration provides a total daily dose of about 250 mg or less, or of about 3 mg/kg or less; or an average total daily dose of about 250 mg or less, or of about 3 mg/kg or less, where the dosage indicates the amount of active ingredient (bucillamine). Accordingly, the invention provides methods of administering a therapeutically effective amount of bucillamine to achieve a therapeutic effect in a subject with gout, a condition relating thereto, or a symptom thereof, by administering a pharmaceutical composition comprising bucillamine, in a treatment dose, and a pharmaceutically acceptable carrier; re-administering said pharmaceutical composition about two to about six hours after said first administration; and repeating these administering steps to provide a total daily dose of 250 mg or less, or of 3 mg/kg/day or less, (or an average total daily dose of 250 mg or less, or of 3 mg/kg/day or less), such as about 200 mg or less, about 150 mg or less, about 120 mg or less, about 100 mg or less, about 75 mg or less, or about 50 mg, as the total daily dose or average total daily dose, and/or as low as about 120 mg/day, about 100 mg/day, about 75 mg/day, about 50 mg/day, about 30 mg/day, or about 25 mg/day.

In some embodiments, the composition is an immediate-release composition for oral administration. In some embodiments, the composition is provided as an immediate-release transdermal or transmucosal formulation. For buccal or sublingual delivery, in some embodiments, bucillamine is embedded in a dissolving wafer placed either between the teeth and cheek or under the tongue, respectively, allowing for easy repeated administration. In preferred embodiments in the context of treating cystinuria, a related disorder, three wafers are administered approximately eight hours apart for a 24-hour period, to deliver a total daily therapeutic dose of bucillamine in repeated pulses. In a particularly preferred embodiment, six wafers are administered approximately four hours apart over a 24 hour period, to deliver a total daily therapeutic dose of bucillamine in repeated pulses, thereby achieving higher and more consistently higher urinary concentration of bucillamine, than more or less frequent dosing schedules. The present dosing schedules differ from those prescribing administration a given number of times a day, which do not specify intervals of time between consecutive administrations.

In preferred embodiments for treating/preventing inflammatory conditions, like gout or rheumatoid arthritis, five wafers are administered approximately five hours apart for a 24-hour period, to deliver a total daily therapeutic dose of bucillamine in repeated pulses. In a particularly preferred embodiment, six wafers are administered approximately four hours apart over a 24 hour period, to deliver a total daily therapeutic dose of bucillamine in repeated pulses, thereby achieving higher and more consistently higher plasma concentrations of free bucillamine, than more or less frequent dosing schedules. The present dosing schedules differ from those prescribing administration a given number of times a day, which do not specify intervals of time between consecutive administrations.

Kits for Use with Modified-Release Compositions and Systems

Yet another aspect of the invention provides kits for use with the compositions, systems, and methods described herein. In some embodiments, the kit provides one or more compositions or systems described herein for use in a method of the invention. For example, kits may provide dosage forms, grouped by doses to be taken at a particular time, and over a day; and/or organized to aid compliance with a particular dosing regimen, throughout a period of time, e.g., for a day, a week, a month, or a year of treatment. Kits for administering the compositions or delivery systems of the invention may also include instructions for use.

It will be understood that the following examples and embodiments described herein are for illustrative purposes and that various modifications or changes in light thereof will be suggested to persons skilled in the art and are to be included within the spirit and purview of this application and scope of the appended claims.

All publications, patents, and published patent applications cited herein are hereby incorporated by reference in their entireties for all purposes.

### EXAMPLES

### Example 1

### Dose Determination for Immediate-Release Bucillamine

This Example describes a study to establish an effective dose of non-coated bucillamine for cystinuria patients, by measuring urine cystine levels.

Study Design: The study identifies patients that are either “good responders” to Thiola®, related thiol containing molecule with FDA approval for the same indication, or “fair responders” to Thiola®, for a total of 8 cystinuria patients. Good responders are people who achieve 250 mg/L or less cystine urine concentration with tiopronin usage, for example, having a baseline without tiopronin of 1,200 mg/L, which falls to 250 mg/L following tiopronin treatments. Fair responders are people who achieve close to but not less than 250 mg/L cystine urine concentration with tiopronin usage, for example, having a baseline without tiopronin of 1,200 mg/L, which only falls to 300 mg/L even following treatments with tiopronin. Another example of a fair responder is a person having a baseline cystine urine concentration of 2,000 mg/L, which only falls to 300 mg/L after treatments with tiopronin. The 8 patients are dosed as follows, depending on results after two days of each dosing schedule:

Day 1: 100 mg of bucillamine is administered orally 3 times a day; also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

If urine cystine is not reduced to 250 mg/L or less, even for “good responders,” after Day 2, the trial is continued as follows:

Day 5: 200 mg of bucillamine is administered orally 3 times a day; also, 24 hour urine cystine capacity is measured, that is, all urine over a 24-hour period, starting from the time of the first dose, is collected and used to measure urinary cystine concentration.

Day 6: Repeat Day 5

Day 7: No treatment

Day 8: No treatment

If urine cystine is not reduced to 250 mg/L or less, even for “good responders,” after Day 6, the trial is continued as follows.

Day 9: 300 mg of bucillamine is administered orally 3 times a day; also, 24 hour urine cystine capacity is measured, that is, all urine over a 24-hour period, starting from the time of the first dose, is collected and used to measure urinary cystine concentration.

Day 10: Repeat Day 9

Day 11: No treatment

Day 12: No treatment

If urine cystine is not reduced to 250 mg/L or less, even for “good responders,” after Day 10, the trial is continued as follows.

Day 13: 400 mg of bucillamine is administered orally 3 times a day; also, 24 hour urine cystine capacity is measured, that is, all urine over a 24-hour period, starting from the time of the first dose, is collected and used to measure urinary cystine concentration.

Day 14: Repeat Day 13

Day 15: No treatment

Day 16: No treatment

### Example 2A

Cystine Concentration in Urine of Cystinuria Patients Over 2-Day Oral Administration of Extended-Release Bucillamine (Simulated) Compared with that Over 2-Day Dosing with Uncoated Bucillamine

This Example describes a study to confirm the feasibility of reducing cystine levels in the urine of cystinuria patients receiving more frequent and lower doses of bucillamine, in a dosing schedule designed to simulate the extended release formulations of the present invention, compared to urine cystine levels in cystinuria patients receiving non-coated bucillamine.

Study Design: The study identifies and groups patients as “good responders” to Thiola® (Group I) and “poor responders” to Thiola® (Group II), each having 4 cystinuria patients. Good responders are people who achieve 250 mg/L or less cystine urine concentration with tiopronin usage, for example, having a baseline without tiopronin of 1,200 mg/L, which falls to 250 mg/L following tiopronin treatments. Poor responders are people who do not achieve 250 mg/L or less cystine urine concentration with tiopronin usage, for example, having a baseline without tiopronin of 1,200 mg/L, which only falls to 1,000 mg/L even following treatments with tiopronin. Another example of a poor responder is a person having a baseline cystine urine concentration of 2,000 mg/L, which only falls to 1,000 mg/L after treatments with tiopronin. The patients of each Group are dosed as follows:

Day 1: 800/3 mg of bucillamine is administered orally 3 times a day (not including a night dose); also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

Day 5: To simulate extended-release, enterically-coated bucillamine is orally administered at 100 mg x 8 times/day (i.e., every three hours). A simple enteric coating is used that avoids release in the stomach. Also, 24 hour urine cystine capacity is measured, that is, all urine over a 24-hour period, starting from the time of the first dose, is collected and used to measure urinary cystine concentration.

Day 6: Repeat Day 5

If effective, results will be as follows. Urine cystine levels will be consistently lower over Days 5-6 (using a simulated extended-release dosing regimen) compared with those over Days 1-2 of immediate-release bucillamine dosing. Further, plasma levels of the drug will be consistently lower over Days 5-6 (using a simulated extended-release dosing regimen) compared with those over Days 1-2 of immediate-release bucillamine dosing. For example, the cystine concentration in the urine may remain within a range of 100 mg/L to 250 mg/L, demonstrating more consistent cystine excretion, without periods of accumulation.

### Example 2B

Cystine Concentration in Urine of Cystinuria Patients Over 2-Day Oral Administration of Extended-Release Bucillamine (Simulated) Compared with that Over 2-Day with Uncoated Bucillamine

This Example describes a second study to confirm the feasibility of reducing cystine levels in the urine of cystinuria patients receiving more frequent and lower doses of bucillamine, in a dosing schedule designed to simulate the extended release formulations of the present invention, compared to urine cystine levels in cystinuria patients receiving non-coated bucillamine.

Study Design: The study identifies and groups patients as “good responders” to Thiola® (Group I) and “poor responders” to Thiola® (Group II), each having 4 cystinuria patients. Good responders are people who achieve 250 mg/L or less cystine urine concentration with tiopronin usage, for example, having a baseline without tiopronin of 1,200 mg/L, which falls to 250 mg/L following tiopronin treatments. Poor responders are people who do not achieve 250 mg/L or less cystine urine concentration with tiopronin usage, for example, having a baseline without tiopronin of 1,200 mg/L, which only falls to 1,000 mg/L even following treatments with tiopronin. Another example of a poor responder is a person having a baseline cystine urine concentration of 2,000 mg/L, which only falls to 1,000 mg/L after treatments with tiopronin. The patients of each Group are dosed as follows:

Day 1: 400 mg of bucillamine is administered orally 3 times a day (not including a night dose); also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

Day 5: To simulate extended-release, enterically-coated bucillamine is orally administered at 100 mg x 12 times/day (i.e., every two hours). A simple enteric coating is used that avoids release in the stomach. Also, 24 hour urine cystine capacity is measured, that is, all urine over a 24-hour period, starting from the time of the first dose, is collected and used to measure urinary cystine concentration.

Day 6: Repeat Day 5

If effective, results will be as follows. Urine cystine levels will be consistently lower over Days 5-6 (using a simulated extended-release dosing regimen) compared with those over Days 1-2 of immediate-release bucillamine dosing. Further, plasma levels of the drug will be consistently lower over Days 5-6 (using a simulated extended-release dosing regimen) compared with those over Days 1-2 of immediate-release bucillamine dosing. For example, the cystine concentration in the urine may remain within a range of 100 mg/L to 250 mg/L, demonstrating more consistent cystine excretion, without periods of accumulation.

### Example 2C

Cystine Concentration in Urine of Cystinuria Patients Over 2-Day Oral Administration of Extended-Release Bucillamine (Simulated) Compared with that Over 2-Day Dosing with Uncoated Bucillamine

This Example describes a study to confirm the feasibility of reducing cystine levels in the urine of cystinuria patients receiving more frequent and lower doses of bucillamine, in a dosing schedule designed to simulate the extended release formulations of the present invention, compared to urine cystine levels in cystinuria patients receiving non-coated bucillamine.

Study Design: The study identifies and groups patients as “good responders” to Thiola® (Group I) and “poor responders” to Thiola® (Group II), each having 4 cystinuria patients. Good responders are people who achieve 250 mg/L or less cystine urine concentration with tiopronin usage, for example, having a baseline without tiopronin of 1,200 mg/L, which falls to 250 mg/L following tiopronin treatments. Poor responders are people who do not achieve 250 mg/L or less cystine urine concentration with tiopronin usage, for example, having a baseline without tiopronin of 1,200 mg/L, which only falls to 1,000 mg/L even following treatments with tiopronin. Another example of a poor responder is a person having a baseline cystine urine concentration of 2,000 mg/L, which only falls to 1,000 mg/L after treatments with tiopronin. The patients of each Group are dosed as follows:

Day 1: 200 mg of bucillamine is administered orally 3 times a day (not including a night dose); also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

Day 5: To simulate extended-release, enterically-coated bucillamine is orally administered at 50 mg x 12 times/day (i.e., every two hours). The 50 mg dose is obtained by breaking a 100 mg tablet in half. A simple enteric coating is used that avoids release in the stomach. Also, 24 hour urine cystine capacity is measured, that is, all urine over a 24-hour period, starting from the time of the first dose, is collected and used to measure urinary cystine concentration.

Day 6: Repeat Day 5

If effective, results will be as follows. Urine cystine levels will be consistently lower over Days 5-6 (using a simulated extended-release dosing regimen) compared with those over Days 1-2 of immediate-release bucillamine dosing. Further, plasma levels of the drug will be consistently lower over Days 5-6 (using a simulated extended-release dosing regimen) compared with those over Days 1-2 of immediate-release bucillamine dosing. For example, the cystine concentration in the urine may remain within a range of 100 mg/L to 250 mg/L, demonstrating more consistent cystine excretion, without periods of accumulation.

### Example 2D

Cystine Concentration in Urine of Cystinuria Patients Over 2-Day Oral Administration of Extended-Release Bucillamine (Simulated) Compared with that Over 2-Day Dosing with Uncoated Bucillamine

This Example describes a study to confirm the feasibility of reducing cystine levels in the urine of cystinuria patients receiving more frequent and lower doses of bucillamine, in a dosing schedule designed to simulate the extended release formulations of the present invention, compared to urine cystine levels in cystinuria patients receiving non-coated bucillamine.

Study Design: The study identifies and groups patients as “good responders” to Thiola® (Group I) and “poor responders” to Thiola® (Group II), each having 4 cystinuria patients. Good responders are people who achieve 250 mg/L or less cystine urine concentration with tiopronin usage, for example, having a baseline without tiopronin of 1,200 mg/L, which falls to 250 mg/L following tiopronin treatments. Poor responders are people who do not achieve 250 mg/L or less cystine urine concentration with tiopronin usage, for example, having a baseline without tiopronin of 1,200 mg/L, which only falls to 1,000 mg/L even following treatments with tiopronin. Another example of a poor responder is a person having a baseline cystine urine concentration of 2,000 mg/L, which only falls to 1,000 mg/L after treatments with tiopronin. The patients of each Group are dosed as follows:

Day 1: 200 mg of bucillamine is administered orally 3 times a day (not including a night dose); also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

Day 5: To simulate extended-release, enterically-coated bucillamine is orally administered at 100 mg x 6 times/day (i.e., every four hours). A simple enteric coating is used that avoids release in the stomach. Also, 24 hour urine cystine capacity is measured, that is, all urine over a 24-hour period, starting from the time of the first dose, is collected and used to measure urinary cystine concentration.

Day 6: Repeat Day 5

If effective, results will be as follows. Urine cystine levels will be consistently lower over Days 5-6 (using a simulated extended-release dosing regimen) compared with those over Days 1-2 of immediate-release bucillamine dosing. Further, plasma levels of the drug will be consistently lower over Days 5-6 (using a simulated extended-release dosing regimen) compared with those over Days 1-2 of immediate-release bucillamine dosing. For example, the cystine concentration in the urine may remain within a range of 100 mg/L to 250 mg/L, demonstrating more consistent cystine excretion, without periods of accumulation.

### Example 3

Cystine Stone Formation in Cystinuria Patients Following Oral Administration of Extended-Release Bucillamine (Simulated) for about 1 Week

This Example describes a study to confirm the improved efficacy of reducing cystine stone formation in cystinuria patients who develop at least one stone per week using standard Thiola® dosing.

Study Design: The study identifies 2-3 patients currently taking Thiola® but who still develop at least one cystine stone per week. The patients are dosed as follows:

Day 1: No treatment; an X-ray is taken of the kidney and bladder.

Days 2-8: To simulate extended release, enterically-coated bucillamine is orally administered at either 100 mg x 8 times/day (i.e., every three hours) or 100 mg x 12 times/day (i.e., every two hours). A simple enteric coating is used that avoids release in the stomach. Also, 24 hour urine cystine capacity is measured, that is, all urine over a 24-hour period, starting from the time of the first dose, is collected and used to measure urinary cystine concentration.

Day 8: X-ray examination of Day 1 is repeated.

If effective, results will be as follows. The X-ray of Day 8 will show no additional stones in addition to those, if any, observed on Day 1 for each patient in the study, or for one or more patients in the study.

### Example 4

Cystine Stone Formation in Cystinuria Patients Following Oral Administration of Extended-Release Bucillamine (Simulated) for about 1 Month

This Example describes a study to confirm the improved efficacy of reducing cystine stone formation in cystinuria patients who develop at least one stone, e.g., about 2-3 stones, per month using standard Thiola® dosing.

Study Design: The study identifies 2-3 patients currently taking Thiola® but who still develop about 2 to 3 stones a month. The patients are dosed as follows:

Day 1: No treatment; an X-ray is taken of the kidney and bladder.

Days 2-31: To simulate extended release, enterically-coated bucillamine is orally administered at either 100 mg x 8 times/day (i.e., every three hours) or 100 mg x 12 times/day (i.e., every two hours). A simple enteric coating is used that avoids release in the stomach. Also, 24 hour urine cystine capacity is measured, that is, all urine over a 24-hour period, starting from the time of the first dose, is collected and used to measure urinary cystine concentration.

Day 31: X-ray examination of Day 1 is repeated.

If effective, results will be as follows. The X-ray of Day 31 will show no additional stones in addition to those, if any, observed on Day 1 for each patient in the study, or for one or more patients in the study; or will show only 1-2 additional stones compared to Day 1, for each patient in the study or for one or more patients in the study.

### Example 5

Cystine Concentration in Urine of Cystinuria Dogs Over 2-Day Oral Administration of Extended-Release Bucillamine (Simulated) Compared with that Over 2-Day Uncoated Bucillamine

This Example describes a study to confirm the feasibility of reducing cystine levels in the urine of cystinuria dogs receiving more frequent and lower doses of bucillamine, in a dosing schedule designed to simulate the extended release formulations of the present invention, compared to urine cystine levels in cystinuria dogs receiving non-coated bucillamine.

Study Design: Subject dogs are dosed as follows:

Day 1: A suitable dose of bucillamine is administered orally 2 times a day (dosage schedule for a tiopronin administration to dogs, which does not include a night dose); also, 24 hour urine cystine capacity is measured, that is, all urine over a 24-hour period, starting from the time of the first dose, is collected and used to measure urinary cystine concentration.

Day 2: Repeat Day 1

Day 3: No treatment

Day 4: No treatment

Day 5: To simulate extended-release, enterically-coated bucillamine is orally administered at lower dose, 8 times/day (i.e., every three hours), to provide a total daily dosage equal to that used in Days 1 and 2. A simple enteric coating is used that avoids release in the stomach. Also, 24 hour urine cystine capacity is measured, that is, all urine over a 24-hour period, starting from the time of the first dose, is collected and used to measure urinary cystine concentration.

Day 6: Repeat Day 5

If effective, results will be as follows. Urine cystine levels will be consistently lower over Days 5-6 (using a simulated extended-release dosing regimen) compared with those over Days 1-2 of uncoated (immediate-release) bucillamine dosing. Further, plasma levels of the drug will be consistently lower over Days 5-6 (using a simulated extended-release dosing regimen) compared with those over Days 1-2 of uncoated (immediate-release) bucillamine dosing.

### Example 6

### Total and Free Bucillamine Concentration in Plasma and Bucillamine Recovery in Urine of Dogs Following Oral and Intraduodenal Administration According to Dosing Schedules Simulating Modified-Release Bucillamine

The objective of this study was to evaluate total and free plasma bucillamine, and urinary bucillamine, in male Beagle dogs following oral and intraduodenal administration of bucillamine, according to various dose regimens, to determine whether different regimens, simulating modified release in accordance with the present invention, would improve drug pharmacokinetics. Specifically, bucillamine formulations were administered to deliver 30 mg/kg per dog via single or multiple doses divided over a 12-hour period; and blood and urine samples then collected at different times post-dose.

These measurements allow an evaluation of the different dosing regimens, and thus of the modified-release formulations and systems described herein, in treating cystinuria and inflammatory conditions (such as gout and arthritis). The following demonstrates a bucillamine delivery approach that surprisingly maintains high unbound bucillamine levels in the urine, the most therapeutically relevant pharmacokinetic parameter for cystinuria. Indeed, an ideal pharmacokinetic profile is one that affords high levels of unbound drug in the urine. Meanwhile a different delivery approach was discovered that surprisingly maintains a moderately high and steady level of unbound bucillamine in plasma with low urinary excretion, which is an ideal pharmacokinetic profile for conditions like gout and arthritis.

Study Design

Bucillamine formulations were administered to provide 30 mg/kg per dog according to five different dosage regimens (Leg 1, Leg 2, Leg 3, Leg 4, and Leg 5), as set forth in the table below. Leg 2, Leg 3, and Leg 4 (also referred to as Q6H, Q3H, and Q2H, respectively) were designed to simulate modified-release formulations, in accordance with certain embodiments of the present invention. Following administration, blood and urine samples were collected at the time points, or over the time intervals, also as indicated below.

TABLE 1

| | | | | | | | Blood | Urine |
|---|---|---|---|---|---|---|---|---|
| | | | | | | | Sampling | Sampling |
| Leg | Test | Dosing | | Dose | | Dose | Time Points | Time Points |
| # | Article | Route | N= | (mg/kg) | Formulation | Regimen | (hours) | (hours) |
| 1 | Bucillamine | PO | 3 | 30 | Bucillamine | Single | 2 hours pre- | 2 hours pre- |
| | | | | | 40%, Mannitol | Dose | dose, 1, 2, 3, 4, | dose, 0-4, 4- |
| | | | | | 30%, Avicel | 0 hr | 5, 6, 7, 8, 9, 10, | 8, 8-12, and |
| | | | | | 30% | (Q12H) | 11, 12, and 24 | 12-24 hours |
| Minimum 7 Days Washout | | | | | | | hours post dose | |
| 2 | Bucillamine | PO | 3 | 15 | Bucillamine | Two | | |
| | | | | | 40%, Mannitol | Doses | | |
| | | | | | 30%, Avicel | 0 and 6 hr | | |
| | | | | | 30% | (Q6H) | | |
| Minimum 7 Days Washout | | | | | | | | |
| 3 | Bucillamine | PO | 3 | 7.5 | Bucillamine | Four | | |
| | | | | | 20%, Mannitol | Doses | | |
| | | | | | 40%, Avicel | 0, 3, 6, | | |
| | | | | | 40% | and 9 hr. | | |
| | | | | | | (Q3H) | | |
| Minimum 7 Days Washout | | | | | | | | |
| 4 | Bucillamine | PO | 3 | 5 | Bucillamine | Six Doses | | |
| | | | | | 20%, Mannitol | 0, 2, 4, 6, | | |
| | | | | | 40%, Avicel | 8, and 10 | | |
| | | | | | 40% | hr. | | |
| | | | | | | (Q2H) | | |
| Minimum 7 Days Washout | | | | | | | | |
| 5 | Bucillamine | ID | 3 | 30 | Bucillamine | Single | | |
| | | | | | 40%, Mannitol | Dose | | |
| | | | | | 30%, Avicel | 0 hr | | |
| | | | | | 30% | (Q12H) | | |

Preparation of Dosing Formulations: Capsules for Legs 1-5 were prepared and filled on the day of dosing, as follows. For Legs 1, 2, and 5, bucillamine was dry mixed with avicel and mannitol to make a 40:30:30 mixture of bucillamine to avicel to mannitol, respectively; for study Legs 3 and 4, a 20:40:40 mixture of bucillamine to avicel to mannitol, respectively, was prepared. The dry mixes were custom filled with the appropriate amount of powder to deliver the specified doses to each dog based, on their respective body weights.

Animal Dosing and Sample Collection: This non-clinical study followed established practices and standard operating procedures of Absorption Systems and Absorption Systems Inc., as well as the study protocol. Study groups each included three dogs. The dogs were housed one per cage. For Legs 1-4, the dogs were fasted for at least six hours prior to each administration of the formulation. For Legs 1-4, food was given approximately ten hours post initial dosing. Animals had free access to water throughout the study. For Leg 5, the dogs were fasted for at least sixteen hours prior to each administration of the formulation. For Leg 5, food was given approximately eight hours post initial dosing. Animals had free access to water throughout the study. Additionally, each dog received a 60 mL subcutaneous injection of saline (a 30 mL injection, into each two different sites) approximately 30 minutes prior to dosing, for hydration.

Intraduodenal (ID) administration: Animals were anesthetized by a slow IV infusion of Propofol (6-10 mg/kg), or with an intramuscular injection of a cocktail containing ketamine (approximately 10-20 mg/kg), diazepam (3 mg/kg), and acepromazine (0.05 mg/kg) given IM about 5-10 minutes before intubation. Once animals were confirmed adequately anesthetized, they were intubated and maintained using isoflurane (approximately 1-5% in oxygen 1 to 4 L/min) as necessary until dosing was complete. Formulations were administered into the proximal duodenum via endoscopic guidance to verify the dosing site. The endoscope first was maneuvered into the pyloric sphincter with camera visualization of the proximal duodenum. Once there was confirmation that the endoscope was placed between the pylorus and proximal duodenum, the test article was delivered to the proximal duodenum followed by a 5 mL flush. Upon completion of the dosing, the animals were taken off the isoflurane and allowed to recover from anesthesia. Time points began upon completion of the entire dose procedure. Following each blood sampling time point, 15 mL of water was given to each dog via syringe. Approximately 30 minutes prior to the 0 hour blood collection, each dog received a subcutaneous injection of 60 mL of saline (a 30 mL injection into each of two different sites) for hydration.

Following dosing, blood and urine samples were collected according to the schedule in Table 1.

Blood samples were collected via direct venipuncture of the jugular vein or other accessible vein of the dog and placed in chilled polypropylene tubes containing sodium heparin as an anticoagulant. Samples were maintained chilled throughout processing. Blood samples were centrifuged at 4° C. and 3,000 rpm (revolutions per minute) for 5 minutes. The first aliquot of plasma was precipitated with acetonitrile by combining 200 μL of plasma with 600 μL of acetonitrile in a centrifuge tube. The samples were mixed, centrifuged at 3,000 rpm for 5 minutes, and the resulting supernatant was decanted into clean, labeled polypropylene tubes. The remaining plasma volume was transferred to a chilled, labeled polypropylene tube, placed on dry ice, and stored in a freezer maintained at −60° C. to −80° C. Following the taking of each blood sample, 15 mL of water was given to the dog via syringe, for hydration.

Pre-dose urine samples were collected via catheter approximately 2 hours pre-dose. Other urine samples were collected via free catch in the cages during the indicated interval, collected into clean bottles, and kept on cold packs. Cold packs were replenished as needed during the day. At the end of each interval, the total volume of urine was collected and recorded. Each sample was well mixed and divided into two aliquots: a first aliquot of approximately 5 mL, and a second aliquot of the remaining volume. If urine was not present in the animal's cage at the end of the final collection interval, the final urine sample was collected by catheter. All urine samples were stored in a freezer maintained at −60° C. to −80°.

Sample Analysis: Plasma concentrations of total and free bucillamine were determined by an LC-MS/MS method; urinary total bucillamine also was determined by LC-MS/MS method, following by HPLC and mass spectrometry.

A specific LC-MS/MS method for determining bucillamine in plasma and urine of Beagle dogs was developed for this study. The method was developed to distinguish total bucillamine and free (not protein bound) bucillamine, since bucillamine has a propensity to bind with protein thiols, other small molecule thiols, or with other bucillamine molecules.

For the analysis, analytical stock solutions (1.00 mg/mL of the free drug) were prepared in DMSO. Also, three standards for use in the analysis were prepared in each of the following media, as follows: (1) in male Beagle plasma containing sodium heparin as the anticoagulant; (2) in Beagle plasma that had previously been treated with acetonitrile to precipitate and remove plasma proteins (the plasma-free supernatant was diluted with acetonitrile (3:1 acetonitrile:plasma supernatant) to give pre-crashed Beagle plasma supernatant); and (3) in 10× diluted Beagle urine, diluted in water. The term “pre-crashed plasma” refers to plasma supernatant, obtained in the centrifugation step described above.

For plasma and urine calibration standards, working solutions were prepared in 50:50 acetonitrile:water. Working solutions were then added to plasma to make calibration standards to final concentrations of 1000, 500, 100, 50.0, 10.0, 5.00, 2.50 and 1.00 ng/mL. Standards in pre-crashed plasma were prepared by serial dilution into the plasma supernatant matrix to the same final concentrations. Samples were manually prepared for analysis in a 96-well plate format. Table 2 summarizes steps of the procedure for analyzing total or free plasma bucillamine, or total urinary bucillamine.

TABLE 2

| Step | Procedure Followed |
|---|---|
| 1 | For Standards: Add 10 μL of appropriate working solution to 50 μL of blank plasma in a |
| | 96 well plate. |
| | For Samples for determining Total Plasma Bucillamine, Urine Bucillamine, and Controls |
| | (Blanks): Aliquot 50 μL of plasma sample or blank plasma in a 96 well plate. Add 10 μL |
| | 50:50 acetonitrile: water containing 10 mM DTT to each sample. |
| | For Samples for determining Free Plasma Bucillamine and Controls (Blanks): Aliquot |
| | 150 μL of sample supernatant or blank supernatant in a 96 well plate. Add 10 μL 50:50 |
| | acetonitrile: water containing 10 mM DTT to each sample. |
| | Cap and mix |
| 2 | Add 20 μL of 1M DTT to each sample and standard. Mix plate for 5 minutes. |
| 3 | For determining Total Plasma Bucillamine: Add 150 μL of 300 ng/mL Bucillamine-d₃ as |
| | an internal standard. Cap and vortex well. |
| | For determining Free Plasma Bucillamine: Add 20 μL of 300 ng/mL Bucillamine-d₃ as |
| | an internal standard. Cap and vortex well. |
| 4 | Cap plate and mix well for 5 minutes. Centrifuge plate at 3000 rpm for 5 minutes. |
| 5 | Transfer and inject supernatant into HPLC. |

It is noted that drug measured in the urine is a measure of free, that is, unbound, bucillamine; as there is typically no protein in urine, there is no appreciable bound drug in the urine. Accordingly, any references to urinary drug concentrations or levels, including references to “total drug” or “total bucillamine” in the urine, refers to free/unbound drug, that is, free/unbound bucillamine, concentraions or levels in the urine.

The HPLC conditions use were as follows:

Instrument: Waters Acquity UPLC

Column: Waters Acquity HSS T3, 50×2.1 mm id, 1.7 μm

Aqueous Reservoir (A): 0.1% Formic Acid in Water

Organic Reservoir (B): 0.1% Formic Acid in Acetonitrile

Gradient Program: As indicated in Table 3.

TABLE 3

| Time | Grad. | | |
|---|---|---|---|
| (min) | Curve | % A | % B |
| 0.00 | 6 | 99.9 | 0.1 |
| 0.75 | 6 | 0.1 | 99.9 |
| 0.80 | 6 | 99.9 | 0.1 |
| 1.00 | 6 | 99.9 | 0.1 |

Flow Rate: 800 μL/min

Injection Volume: 10 μL

Run Time: 1.0 min

Column Temperature: 40° C.

Sample Temperature: 8° C.

Strong Autosampler Wash: 1:1:1(v:v:v) water:methanol:isopropanol with 0.2% formic acid

Weak Autosampler Wash: 4 mM ammonium formate

Following HPCL, the samples were subject to mass spectrometry to confirm their compositions. The Mass Spectrometer Conditions were as follows:

Instrument: PE Sciex API4000

Interface: Electrospray (“Turbo Ion Spray”)

Mode: Multiple Reaction Monitoring (MRM)

Gases: CUR 30, CAD 10, GS1 50, GS2 50

Source Temperature: 500° C.

Voltages and Ions Monitored: As indicated in Table 4 (all settings are in volts)

TABLE 4

| | | Precursor | Product | | | | | |
|---|---|---|---|---|---|---|---|---|
| Analyte | Polarity | Ion | Ion | IS | DP | EP | CE | CXP |
| Bucillamine | Negative | 222.0 | 144.1 | −4500 | −38 | −10 | −20 | −9 |
| Bucillamine-d6 (Internal | Negative | 228.1 | 150.0 | −4500 | −38 | −10 | −20 | −9 |
| Standard) | | | | | | | | |

The following abbreviations are used: IS: Ion Spray Voltage; DP: Declustering Potential; EP: Entrance Potential; CE: Collision Energy; CXP: Collision Cell Exit Potential. Pharmacokinetic analysis was conducted by a non-compartmental model using Phoenix WinNonlin v.8.0 software.

Stability Testing: Finally, short-term and long-term stability of bucillamine in Beagle dog plasma, pre-crashed plasma (plasma supernatant), and urine were assessed using two bucillamine concentrations (50 ng/mL or 500 ng/mL) under each of the following two conditions: (1) storage on the bench top for 4 hours and (2) frozen at −80° C. for up to one month. Samples were considered stable if their concentrations were within ±20% of nominal. These samples met acceptable criteria for stability and results are presented below, in Tables 5, 6, and 7, showing the % bucillamine remaining in Beagle dog plasma (Table 5), plasma supernatant (Table 6), and urine (Table 7).

TABLE 5

| | | % Bucillamine Remaining in Beagle Dog Plasma | |
|---|---|---|---|
| High or Low Bucillamine Concentration | | 50 ng/mL (low) | 500 ng/mL(high) |
| Bench top (RT) for 4 hours | | 110 | 96.9 |
| −80° C. Freezer for: | 1 week | 114 | 96.4 |
| | 2 weeks | 108 | 97.3 |
| | 4 weeks | 114 | 107 |

TABLE 6

| | | % Bucillamine Remaining in | |
|---|---|---|---|
| | | Beagle Dog Plasma Supernatant | |
| High or Low Bucillamine Concentration | | 50 ng/mL (low) | 500 ng/mL (high) |
| Bench top (RT) for 4 hours | | 105 | 117 |
| −80° C. Freezer for: | 1 week | 94.3 | 97.2 |
| | 2 week | 99.1 | 110 |
| | 4 week | 107 | 119 |

TABLE 7

| | | % Bucillamine Remaining in Beagle Dog Urine | |
|---|---|---|---|
| High or Low Bucillamine Concentration | | Low (250 ng/mL) | High (2500 ng/mL) |
| Bench top (RT) for 4 hours | | 93.7 | 102 |
| −80° C. Freezer for: | 1 week | 97.7 | 102 |
| | 2 week | 90.9 | 101 |
| | 4 week | 88.9 | 96.3 |

Results

Results of this study surprisingly demonstrate that there is an optimum dosing frequency range, or critical range for interval lengths between doses, in treating cystinuria or cystinuria-related disorders. This range results in both a higher concentration and a greater amount of unbound bucillamine in urine, compared to a dosing schedule with longer or shorter dosing intervals, administrating the same amount of drug over a given period of time. The present results surprisingly demonstrate that optimal pharmacokinetics are obtained using a modified-release approach that provides fractions of the total daily bucillamine dose about every 4 to 8 hours, and preferably about every 6 hours (rather than more or less frequent dosing) to a patient with cystinuria or related disorder. This results in greater amounts of the drug available in the urine to bind greater amounts of cystine. Another surprising advantage is that an equivalent amount of cystine binding can be achieved using less drug, which may result in fewer side effects. Additionally, such high drug urine recovery surprisingly is achieved using oral dosing, while intraduodenal administration resulted in a significantly lower drug urine recovery, such that step-wise release, at critically spaced intervals in the stomach, presents a preferred modified release strategy in treating disorders like cystinuria.

Results of this study also surprisingly demonstrate that there is an optimum dosing frequency range, or critical range for interval lengths between doses, in treating conditions like gout and arthritis. This range results in higher and more steady levels of free (unbound) bucillamine in the plasma, although less drug excreted into the urine, compared with dosing schedules having longer or shorter dosing intervals, administrating the same amount of drug over a given period of time. The present results surprisingly demonstrate that optimal pharmacokinetics are obtained using a modified-release approach that provides fractions of the total daily (or weekly) bucillamine dose about every 2 to 6 hours. (rather than more or less frequent dosing) to a patient with gout, arthritis, or related condition. These pharmacokinetic features are further improved by avoiding small intestine delivery and absorption, similarly as in the case of treating cystinuria patients with bucillamine.

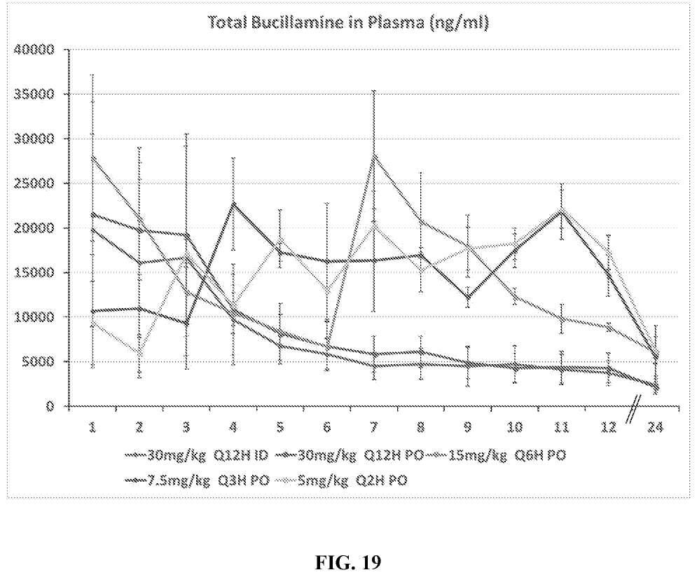

Total Bucillamine Plasma Concentration: Total bucillamine concentration in the plasma was measured as described above. Results for each of Legs 1-5 are provided below and FIG. 19.

Leg 1 (Q12H): Following PO dosing of bucillamine at 30 mg/kg, using a single dose, maximum plasma concentrations of total bucillamine (average of 24400±8253 ng/mL) were observed between 1 and 3 hours post dosing.

Leg 2 (Q611): Following PO dosing of bucillamine at 30 mg/kg, using two 15 mg/kg doses at 0 and 6 h, maximum plasma concentrations (average of 33533±2139 ng/mL) of total bucillamine were observed between 1 and 7 hours post dosing.

Leg 3 (Q3H): Following PO dosing of bucillamine at 30 mg/kg, using four 7.5 mg/kg doses at 0, 3, 6, and 9 h, maximum plasma concentrations (average of 25133±3500 ng/mL) of total bucillamine were observed between 4 and 11 hours post dosing.

Leg 4 (Q611): Following PO dosing of bucillamine at 30 mg/kg, using six 5 mg/kg doses at 0, 2, 4, 6, 8, and 10 h, maximum plasma concentrations (average of 23800±819 ng/mL) of total bucillamine were observed between 7 and 11 hours post dosing.

Leg 5 (Q12H): Following ID dosing of bucillamine at 30 mg/kg, using a single dose, maximum plasma concentrations of total bucillamine (average of 19940±11015 ng/mL) were observed between 1 and 3 hours post dosing.

FIG. 19 shows the total concentration of bucillamine in plasma at various time points post-dosing for the four different oral dosing regimens and the one intraduodenal regimen. Values are averaged; bars represent standard deviation (n=3).

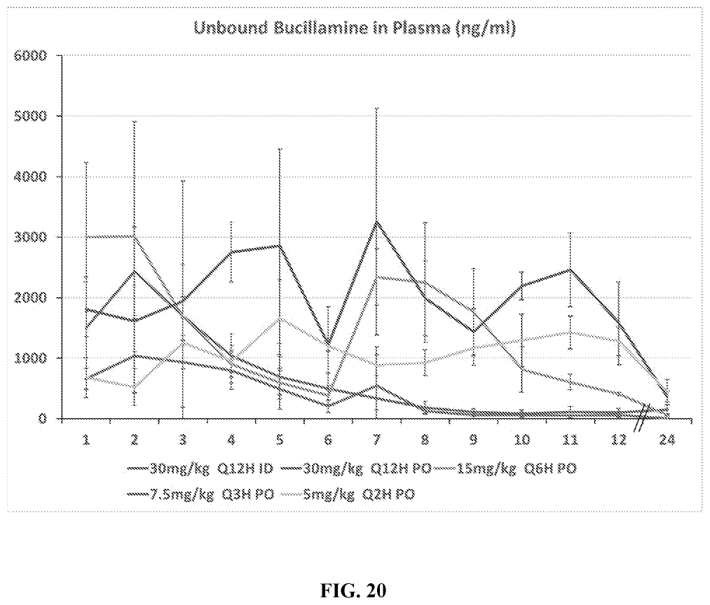

Free Bucillamine Plasma Concentration: Free bucillamine concentration in the plasma was measured as described above. Results for each of Legs 1-5 are provided below and FIG. 20.

Leg 1 (Q12H): After PO dosing of bucillamine at 30 mg/kg, using a single dose, maximum plasma concentrations (average of 2437±728 ng/mL) of free bucillamine were observed at 2 hours post dosing.

Leg 2 (Q611): After PO dosing of bucillamine at 30 mg/k, using two 15 mg/kg doses at 0 and 6 h, maximum plasma concentrations (average of 3957±1226 ng/mL) of free bucillamine were observed between 1 and 7 hours post dosing.

Leg 3 (Q3H): After PO dosing of bucillamine at 30 mg/kg, using four 7.5 mg/kg doses at 0, 3, 6, and 9 h, maximum plasma concentrations (average of 4327±1127 ng/mL) of free bucillamine were observed between 5 and 7 hours post dosing.

Leg 4 (Q611): After PO dosing of bucillamine at 30 mg/kg, using six 5 mg/kg doses at 0, 2, 4, 6, 8, and 10 h, maximum plasma concentrations (average of 1820±471 ng/mL) of free bucillamine were observed between 5 and 11 hours post dosing.

Leg 5 (Q1211): After ID dosing of bucillamine at 30 mg/kg, using a single dose, maximum plasma concentrations (average of 1187±656 ng/mL) of free bucillamine were observed between 2 and 4 hours post dosing.

FIG. 20 shows free (unbound) bucillamine concentration (ng/mL) in plasma at different times post-dosing, using Beagle dogs orally administered 30 mg/kg bucillamine according to 4 dosage regimens a single 30 mg/kg dose (Q12H PO, Leg 1); two 15 mg/kg doses 6 hours apart (Q6H PO, Leg 2); four 7.5 mg/kg doses, every 3 hours (Q3H PO; Leg 3); and six 5 mg/kg doses, every 2 hours (Q2H PO, Leg 4); as well as that following intraduodenal (ID) administration of a single 30 mg/kg dose (Q12H ID, Leg 5). Values are averaged; bars represent standard deviation (n=3 dogs).

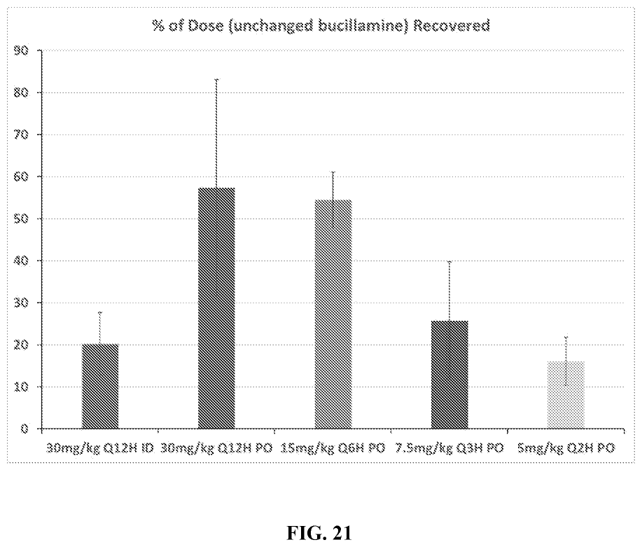

Bucillamine Urinary Concentration: Bucillamine concentration in the urine was measured as described above and reported as % of dose (unchanged drug). As noted above, drug measured in the urine is a measure of free, that is, unbound, bucillamine; as there is typically no protein in urine, there is no appreciable bound drug in the urine. Accordingly, any references to urinary drug concentrations or levels, including references to “total drug” or “total bucillamine” in the urine, refers to free/unbound drug, that is, free/unbound bucillamine, concentrations or levels in the urine. Results for each of Legs 1-5 are provided below and FIG. 21.

Leg 1 (Q1211): Following PO dosing of bucillamine at 30 mg/kg, using a single dose, on average, 57.3±25.8% of the dose (unchanged drug) was found in urine after oral dosing.

Leg 2 (Q611): Following PO dosing of bucillamine at 30 mg/kg, using two 15 mg/kg doses at 0 and 6 h, on average, 54.5±6.63% of the dose (unchanged drug) was found in urine after oral dosing.

Leg 3 (Q3H): Following PO dosing of bucillamine at 30 mg/kg, using four 7.5 mg/kg doses at 0, 3, 6, and 9 h, on average, 25.8±14.0% of the dose (unchanged drug) was found in urine after oral dosing.

Leg 4 (Q611): Following PO dosing of bucillamine at 30 mg/kg, using six 5 mg/kg doses at 0, 2, 4, 6, 8, and 10 h, on average, 16.1±5.65% of the dose (unchanged drug) was found in urine after oral dosing.

Leg 5 (Q1211): Following ID dosing of bucillamine at 30 mg/kg, using a single dose, on average, 20.2±7.51% of the dose (unchanged drug) was found in urine after oral dosing.

FIG. 21 shows urinary recovery of bucillamine, reported as % of dose recovered, for the four different oral dosing regimens and the one intraduodenal regimen. Values are averaged; bars represent standard deviation (n−=3). Of the four oral dosing schedules tested, Q12H and Q611 showed the best bucillamine recovery in urine. Since the formation of cystine uroliths in cystinuria and related disorders is the direct result of saturation of cystine in urine, bucillamine levels in the urine are a therapeutically relevant parameter in demonstrating efficacy, that is, in demonstrating the drug's ability to decrease cystine concentration in the urine via formation of a more soluble the bucillamine-cysteine mixed disulfide, which is then excreted.

It is crucial to maintain a continuous low cystine concentration in urine to prevent cystine seed formation, which grows over time to form cystine stones.

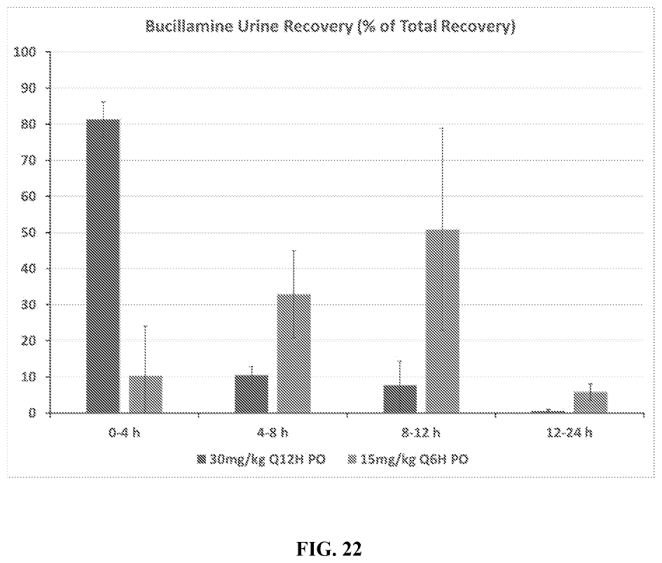

FIG. 22 depicts bucillamine urine recovery per time frames: 0-4 hours, 4-8 hours, 8-12 hours, and 12-24 hours, for a single 30 mg/kg dose (Q12H PO, Leg 1) and two 15 mg/kg doses 6 hours apart (Q6H PO, Leg 2). For this analysis, only dogs that produced a urine sample for each time frame were considered as a result, and two dogs per group were used. Values are averaged in % of total drug dose urine recovery per dosing group. Bars represent standard deviation (n=2 dogs).

FIG. 22 demonstrates that using a Q12H schedule, 80% of the drug recovered in urine surprisingly gets excreted within the first 4 hours, leaving an 8-hour period without sufficient drug to effectively bind cystine. The presence of high cystine concentration in the urine leads to the formation of cysteine stones. In contrast, using a Q611 schedule and providing the same total dose, the amount of drug recovered in the urine surprisingly is spread over an extended period of time, providing a steadier presence of the drug in urine, and hence better maintaining lower cystine urine concentration and its subsequent therapeutic effect.

### DISCUSSION

These results surprisingly demonstrate an optimum dosing frequency range, or critical range for interval lengths between doses, that produces higher and more consistently higher free bucillamine in the urine instead of the blood, that further is improved by avoiding small intestine delivery and absorption (see again, e.g., FIG. 21's Q6H schedule, delivering higher urine recovery of the free drug over time compared to other schedules). Following these strategies will improve efficacy and convenience for cystinuria patients, while reducing side effects. Importantly, this study identifies that critically-spaced, repeated boluses of bucillamine increase urine concentration of bucillamine, as compared to approaches currently used to treat cystinuria, which are based on blood levels of drug, and this study further identifies advantages of stomach delivery and adsorption.

Whereas previous studies compared plasma levels of bound and free drug for patients receiving immediate-release formulations, and it was believed that extended-release formulations might increase availability of free drug in the blood (see, e.g., Koide et al., 1994, “A new therapeutic agent for cystinuria,” Urolithiasis 2:571-574, discussing such a study in the context of tiopronin), the present studies surprisingly demonstrate (1) rather than just extended-release using shorter and shorter dose intervals, there is a critical dose frequency range, or critical range for interval lengths between doses, which (2) surprisingly increases availability of free bucillamine in the urine, rather than just in the plasma; and (3) surprisingly shows that bucillamine administration and absorption in the stomach, rather than in the small intestine, further enhances these desired pharmacokinetic parameters. Simply put, the present work proves a relationship between free drug levels in the blood and free drug levels in urine, where the drug is bucillamine, and further demonstrates surprising administration and dosing schedules to capitalize on this relationship (see, e.g., Q6H). Higher and more consistently higher levels of urinary bucillamine are correlated with improved efficacy in treating cystinuria. That is, higher urinary bucillamine results in lower urinary cystine, thus reducing or eliminating cystine stone formation for subjects with cystinuria. Accordingly, dose-frequency administration parameters shown here to give higher bucillamine urine excretion represent a strategy to lower cystine concentration in urine, thereby enhancing efficacy, as well as patient compliance and overall outcome for cystinuria patients.

The present study further proves a dosing schedule to optimize blood levels of free bucillamine, of importance in treating/preventing inflammatory diseases such as rheumatoid arthritis and gout (see again, e.g., FIG. 20's Q3H schedule, delivering higher plasma free drug levels compared to other schedules). That is, the present work also surprisingly demonstrates a different optimum dosing frequency range, or critical range for interval lengths between doses, corresponding to shorter intervals between doses, that produces higher and steadier free bucillamine concentration in plasma, though lower drug recovery in the urine, while still lowering total bucillamine peak blood levels. These pharmacokinetic features surprisingly also are further improved by avoiding small intestine delivery and absorption, similarly as in the case of treating cystinuria patients with bucillamine.

What is claimed is:

1. A pharmaceutical composition comprising:
a therapeutically effective amount of bucillamine; and
a pharmaceutically acceptable modified-release component for releasing the bucillamine in repeated pulses over time to the gastrointestinal tract and/or bloodstream of a subject,
wherein a first fraction of the bucillamine is released at a first time point and a second fraction of the bucillamine is released at a second time point about four to about eight hours after said first time point.

2. The composition according to claim 1, wherein a third fraction of the bucillamine is released at a third time point about four to about eight hours after said second time point.

3. The composition according to claim 2, wherein a subsequent n^{th} fraction of the bucillamine is released at a subsequent n^{th} time point about four to about eight hours after a preceding (n−1)^{th} time point; and/or wherein said fractions are equal or approximately equal.

4. The composition according to any preceding claim, wherein the bucillamine is released in repeated pulses about every four to eight hours for about 12 to about 24 hours.

5. The composition according to any preceding claim, wherein the therapeutically effective amount is a daily dose of bucillamine and the bucillamine is released in repeated pulses about every six hours for about 24 hours.

6. The composition according to any preceding claim, wherein the composition is formulated for oral administration.

7. The composition according to claim 6, wherein the modified-release component of the oral formulation comprises at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; a timed microprocessor, and a gastro-retentive feature.

8. The composition according to any one of claims 1 to 7, wherein said first and second releases occur in the stomach.

9. The composition according to claim 8, wherein said first, second, and third releases occur in the stomach.

10. The composition according to claim 9, wherein said first, second, third, and fourth releases occur in the stomach.

11. The composition according to any one of claims 8 to 10, wherein said gastro-retentive feature is at least one selected from the group consisting of a stomach-adhering coating, a floatation system, a sedimentation system, and an expandable system.

12. The composition according to claim 11, wherein said composition further comprises an immediate-release component and said first release occurs from said immediate-release component.

13. The composition according to claim 11 or 12, wherein said stomach-adhering coating comprises stomach-adhering polymers that partially coat at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

14. The composition according to claim 11 or 12, wherein said floatation system comprises an effervescent or non-effervescent agent that increases buoyancy of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; and a matrix comprising differently-adhering polymers.

15. The composition according to claim 11 or 12, wherein said sedimentation system comprises a high density agent combined in a pellet with at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

16. The composition according to claim 11 or 12, wherein said expandable system comprises a shape-modifying or swellable structure that impedes exit from the stomach of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

17. The composition according to any one of claims 7 to 16, wherein the layers of coatings of said modified-release component comprise a first coating that releases a first fraction of the bucillamine at a first time point and a second coating that releases a second fraction of the bucillamine at a second time point about four to about eight hours after said first time point, said first and second releases occurring in the stomach.

18. The composition according to claim 17, wherein said layers of coatings further comprise a third coating that releases a third fraction of the bucillamine, in the stomach or duodenum, at a third time point about four to about eight hours after the second time point.

19. The composition according to any one of claims 7 to 16, wherein the matrix of differently-dissolving segments of said modified-release component comprises a first segment that dissolves to release a first fraction of the bucillamine at a first time point and a second segment that dissolves to release a second fraction of the bucillamine at a second time point about four to about eight hours after said first time point, said first and second releases occurring in the stomach.

20. The composition according to claim 19, wherein said matrix of differently-dissolving segments further comprises a third segment that dissolves to release a third fraction of the bucillamine, in the stomach or duodenum, at a third time point about four to about eight hours after the second time point.

21. The composition according to any one of claims 7 to 16, wherein the differently-coated beads of said modified-release component comprise a first bead coating that releases a first fraction of the bucillamine at a first time point and a second bead coating that releases a second fraction of the bucillamine at a second time point about four to about eight hours after said first time point, said first and second releases occurring in the stomach.

22. The composition according to claim 21, wherein the differently-coated beads further comprise a third bead coating that releases a third fraction of the bucillamine, in the stomach or duodenum, at a third time point about four to about eight hours after the second time point.

23. The composition according to any one of claims 7-16, wherein the differently-dissolving bands of said modified-release component comprise a first band that releases a first fraction of the bucillamine at a first time point and a second band that releases a second fraction of the bucillamine at a second time point about four to about eight hours after said first time point, said first and second releases occurring in the stomach.

24. The composition according to claim 23, wherein the differently-dissolving bands further comprise a third band that releases a third fraction of the bucillamine, in the stomach or duodenum, at a third time point about four to about eight hours after the second time point.

25. The composition according to any one of claims 7 to 16, wherein the differently-releasing plugs of said modified-release component comprise a first plug that releases a first fraction of the bucillamine at a first time point and a second plug that releases a second fraction of the bucillamine at a second time point about four to about eight hours after said first time point, said first and second releases occurring in the stomach.

26. The composition according to claim 25, wherein the differently-releasing plugs further comprise a third plug that releases a third fraction of the bucillamine, in the stomach or duodenum, at a third time point about four to about eight hours after the second time point.

27. The composition according to any one of claims 7 to 16, wherein the differently-expanding osmotic-push compartments of said modified-release component comprise a first osmotic-push compartment that releases a first fraction of the bucillamine at a first time point and a second osmotic-push compartment that releases a second fraction of the bucillamine at a second time point about four to about eight hours after said first time point, said first and second releases occurring in the stomach.

28. The composition according to claim 27, wherein the differently-expanding osmotic-push compartments further comprise a third osmotic-push compartment that releases a third fraction of the bucillamine, in the stomach or duodenum, at a third time point about four to about eight hours after the second time point.

29. The composition according to any one of claims 7 to 16, wherein the differently-releasing polymers of said modified-release component comprise a first polymer that releases a first fraction of the bucillamine at a first time point and a second polymer that releases a second fraction of the bucillamine at a second time point about four to about eight hours after said first time point, said first and second releases occurring in the stomach.

30. The composition according to claim 29, wherein the differently-releasing polymers further comprise a third polymer that releases a third fraction of the bucillamine, in the stomach or duodenum, at a third time point about four to about eight hours after the second time point.

31. The composition according to any one of claims 7 to 16, wherein the differently-adhering polymers of said modified-release component comprise a first polymer that releases a first fraction of the bucillamine at a first time point and a second polymer that releases a second fraction of the bucillamine at a second time point about four to about eight hours after said first time point, said first and second releases occurring in the stomach.

32. The composition according to claim 31, wherein the differently-adhering polymers further comprise a third polymer that releases a third fraction of the bucillamine, in the stomach or duodenum, at a third time point about four to about eight hours after the second time point.

33. The composition according to any one of claims 17 to 32, wherein said second time point is about six hours after said first time point.

34. The composition according to any one of claims 1 to 5, wherein the composition is formulated for transdermal delivery or transmucosal delivery.

35. The composition according to claim 34, wherein the transdermal formulation is provided in a dermal patch.

36. The pharmaceutical composition according to any preceding claim in an effective amount for use in treating cystinuria, a disorder related thereto, or a symptom thereof, in a subject in need thereof.

37. A method of treating cystinuria, a disorder related thereto, or a symptom thereof, in a subject in need thereof, comprising administering to the subject an effective amount of the pharmaceutical composition according to any one of claims 1 to 35.

38. A method of administering a therapeutically effective amount of bucillamine to achieve a therapeutic effect in a subject with cystinuria, a disorder relating thereto, or a symptom thereof, the method comprising:
administering to said subject a pharmaceutical composition comprising bucillamine, in a therapeutically effective treatment dose, and a pharmaceutically acceptable carrier;
re-administering to said subject said pharmaceutical composition about four to about eight hours after said first administration; and
repeating said administering steps to provide an average total daily dose of 14 mg/kg or less.

39. A pharmaceutical composition for use in administering a therapeutically effective amount of bucillamine to achieve a therapeutic effect in a subject with cystinuria, a disorder relating thereto, or a symptom thereof,
said composition comprising a pharmaceutically acceptable carrier and bucillamine in a therapeutically effective treatment dose for repeated administration to said subject, in about four to about eight hour intervals, to provide an average total daily dose of 14 mg/kg or less.

40. The method according to claim 38, or pharmaceutical composition for use according to claim 39, wherein said composition is an immediate-release composition for oral administration.

41. The method according to claim 38, pharmaceutical composition for use according to claim 39, wherein said composition further comprises a gastro-retentive feature.

42. The method according to claim 38, or pharmaceutical composition for use according to claim 39, or the method or pharmaceutical composition for use according to claim 40 or 41, wherein said interval is no less than about four hours and said composition is administered no more than about six times a day.

43. The method or pharmaceutical for use according to claim 42, wherein said interval is six hours and said composition is administered four times a day.

44. A method of administering a therapeutically effective amount of bucillamine to achieve an extended duration therapeutic effect in a subject with cystinuria, a disorder relating thereto, or a symptom thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about four to about eight hours;
wherein dosing is repeated to provide an average total daily dose of about 14 mg/kg or less; and
wherein said first treatment dose achieves the extended duration therapeutic effect of reducing urinary cystine concentration effectively below about 250 mg/L in said subject for at least about an 8-hour duration of effect, optionally, before a second or subsequent treatment dose is administered.

45. A method of treating cystinuria, a disorder relating thereto, or a symptom thereof, with extended duration of effect, in a subject in need thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about four to about eight hours;
wherein dosing is repeated to provide an average total daily dose of about 14 mg/kg or less; and
wherein said first treatment dose provides at least about an 8-hour duration of effect of reducing urinary cystine concentration effectively below about 250 mg/L in said subject,
thereby extending treatment interval duration for said subject.

46. A pharmaceutical composition in a formulation for use in administering a therapeutically effective amount of bucillamine to achieve an extended duration therapeutic effect in a subject with cystinuria, a disorder relating thereto, or a symptom thereof,
said composition comprising bucillamine, in a first treatment dose, and a pharmaceutically acceptable modified-release component that releases fractions of the bucillamine in repeated pulses over time at intervals of about four to about eight hours;
wherein dosing is repeated to provide an average total daily dose of about 14 mg/kg or less; and
wherein said first treatment dose achieves the extended duration therapeutic effect in reducing urinary cystine concentration effectively below about 250 mg/L in said subject for at least about an 8-hour duration of effect.

47. A pharmaceutical composition in a formulation for use in treating cystinuria, a disorder relating thereto, or a symptom thereof, with extended duration of effect, in a subject in need thereof, said composition comprising:
bucillamine, in a first treatment dose, and a pharmaceutically acceptable modified-release component that releases fractions of the bucillamine in repeated pulses over time at intervals of about four to about eight hours;
wherein dosing is repeated to provide an average total daily dose of about 14 mg/kg or less; and
wherein said first treatment dose provides at least about an 8-hour duration of effect in reducing urinary cystine concentration effectively below about 250 mg/L in said subject,
thereby extending treatment interval duration for said subject.

48. The method according to claim 44 or claim 45, or the pharmaceutical composition for use according to claim 46 or claim 47, wherein release of at least said first fraction occurs in the stomach.

49. The method or pharmaceutical composition for use according to claim 48, wherein release of at least said first and second fractions occur in the stomach.

50. The method according to claim 44 or claim 45, the pharmaceutical composition for use according to claim 46 or claim 47, or the method or pharmaceutical composition for use according to claim 48 or 49, wherein the composition achieves the extended duration effect for at least about 12 hours.

51. The method or the pharmaceutical composition for use according to claim 50, wherein the composition achieves the extended duration effect for at least about 24 hours.

52. The method according to claim 44 or claim 45, or the pharmaceutical composition for use according to claim 46 or claim 47, or the method or pharmaceutical composition for use according to any one of claims 48 to 50, wherein the treatment dose is administered no more than twice a day.

53. The method or pharmaceutical composition for use according to claim 52, wherein the first treatment dose is 7 mg/kg or less.

54. The method or pharmaceutical composition for use according to claim 53, wherein the first treatment dose is 5 mg/kg or less.

55. The method according to claim 44 or claim 45, or pharmaceutical composition for use according to claim 46 or claim 47, or the method or pharmaceutical composition for use of any one of claims 48 to 54, wherein the composition achieves a peak blood level of total bucillamine of no more than about 30,000 ng/mL.

56. The method or pharmaceutical composition for use according to claim 55, wherein the composition achieves a peak blood level of total bucillamine of no more than about 25,000 ng/mL.

57. The method according to claim 44 or claim 45, or pharmaceutical composition for use according to claim 46 or claim 47, or the method or pharmaceutical composition for use of any one of claims 48 to 56, wherein the composition achieves urinary cystine concentration effectively below about 200 mg/L for the at least about 8 hours of extended duration of effect.

58. The method or pharmaceutical composition for use according to claim 57, wherein the composition achieves urinary cystine concentration effectively below about 200 mg/L for at least about 12 hours of extended duration of effect.

59. The method according to claim 44 or claim 46, or pharmaceutical composition for use according to claim 47 or claim 48, or the method or pharmaceutical composition for use of any one of claims 49 to 58, wherein the composition achieves urinary bucillamine recovery of about 25% to about 75% of the administered bucillamine over about 12 hours of extended duration of effect.

60. The method or pharmaceutical composition for use according to claim 59, wherein the composition achieves urinary bucillamine recovery of about 40% to about 60% of the administered bucillamine over about 12 hours of extended duration of effect.

61. The method according to claim 44 or claim 45, or the pharmaceutical composition for use according to claim 46 or claim 47, or the method or pharmaceutical composition for use according to any one of claims 48 to 60, wherein said composition is formulated for oral delivery.

62. The method or pharmaceutical composition for use according to claim 61, wherein the modified-release component of the oral formulation is at least one selected from the group consisting of: layers of coatings; a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers, matrix comprising differently-adhering polymers, and a gastro-retentive feature.

63. The method or pharmaceutical composition for use according to claim 62, wherein said modified-release component comprises said gastro-retentive feature.

64. The method or pharmaceutical composition for use according to claim 63, wherein said gastro-retentive feature is at least one selected from the group consisting of a stomach-adhering coating, a floatation system, a sedimentation system, and an expandable system.

65. The method according to claim 44 or claim 45, or the pharmaceutical composition for use according to claim 46 or claim 47, or the method or pharmaceutical composition for use of any one of claims 48 to 60, wherein said composition is formulated for transmucosal or transdermal delivery.

66. A method of making the pharmaceutical composition according to any of claims 1 to 35, said method comprising:
providing a therapeutically effective amount of bucillamine; and
providing a pharmaceutically acceptable modified-release component that releases fractions of the bucillamine in repeated pulses over time at intervals of about four to about eight hours, and
combining the bucillamine and the modified-release component to provide an oral, transdermal, or transmucosal formulation.

67. The method according to claim 66, wherein said modified-release component comprises a gastro-retentive feature for combination with the bucillamine to provide said oral formulation.

68. A method of administering a therapeutically effective amount of bucillamine to achieve a reduced peak blood level in a subject with cystinuria, a disorder relating thereto, or a symptom thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about four to about eight hours;
wherein said composition achieves a therapeutically effective blood level of free bucillamine, but a reduced peak blood level of total bucillamine of no more than about 3,000 ng/mL; and
wherein dosing is repeated to provide an average total daily dose of 14 mg/kg or less.

69. A pharmaceutical composition in a formulation for use in administering a therapeutically effective amount of bucillamine to achieve a reduced peak blood level in a subject with cystinuria, a disorder relating thereto, or a symptom thereof,
said composition comprising bucillamine, in a first treatment dose, and a pharmaceutically acceptable modified-release component that releases fractions of the bucillamine in repeated pulses over time at intervals of about four to about eight hours;
wherein said composition achieves a therapeutically effective blood level of free bucillamine, but a reduced peak blood level of total bucillamine of no more than about 3,000 ng/mL; and
wherein dosing is repeated to provide an average total daily dose of 14 mg/kg or less.

70. The method according to claim 68, or the pharmaceutical composition for use according to claim 69, wherein said reduced peak blood level results in fewer side effects than administration of an average total daily dose of about 14 mg/kg of bucillamine in an immediate-release formulation divided amongst three administrations per day.

71. A pharmaceutical composition comprising:
a therapeutically effective amount of bucillamine; and
a pharmaceutically acceptable modified-release component for releasing the bucillamine in repeated pulses over time to the gastrointestinal tract and/or bloodstream of a subject,
wherein a first fraction of the bucillamine is released at a first time point and a second fraction of the bucillamine is released at a second time point about two to about six hours after said first time point.

72. The composition according to claim 71, wherein a third fraction of the bucillamine is released at a third time point about two to about six hours after said second time point.

73. The composition according to claim 72, wherein a subsequent n^{th} fraction of the bucillamine is released at a subsequent n^{th} time point about two to about six hours after a preceding (n−1)^{th} time point; and/or wherein said fractions are equal or approximately equal.

74. The composition according to any one of claims 71 to 73, wherein the bucillamine is released in repeated pulses about every two to six hours for about 12 to about 24 hours.

75. The composition according to any one of claims 71 to 74, wherein the therapeutically effective amount is a daily dose of bucillamine for a given condition and the bucillamine is released in repeated pulses about every four hours for about 24 hours.

76. The composition according to any one of claims 71 to 75, wherein the composition is formulated for oral administration.

77. The composition according to claim 76, wherein the modified-release component of the oral formulation comprising at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; a timed microprocessor, and a gastro-retentive feature.

78. The composition according to any one of claims 71 to 77, wherein said first and second releases occur in the stomach.

79. The composition according to claim 78, wherein said first, second, and third releases occur in the stomach.

80. The composition according to claim 79, wherein said first, second, third, and fourth releases occur in the stomach.

81. The composition according to any one of claims 78 to 80, wherein said gastro-retentive feature is at least one selected from the group consisting of a stomach-adhering coating, a floatation system, a sedimentation system, and an expandable system.

82. The composition according to claim 81, wherein said composition further comprises an immediate-release component and said first release occurs from said immediate-release component.

83. The composition according to claim 81 or 82, wherein said stomach-adhering coating comprises stomach-adhering polymers that coat at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

84. The composition according to claim 81 or 82, wherein said floatation system comprises an effervescent or non-effervescent agent that increases buoyancy of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; and a matrix comprising differently-adhering polymers.

85. The composition according to claim 81 or 82, wherein said sedimentation system comprises a high density agent combined in a pellet with at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

86. The composition according to claim 81 or 82, wherein said expandable system comprises a shape-modifying or swellable structure that impedes exit from the stomach of at least one selected from the group consisting of: layers of coatings, a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers; a matrix comprising differently-adhering polymers; and a timed microprocessor.

87. The composition according to any one of claims 77 to 86, wherein the layers of coatings of said modified-release component comprise a first coating that releases a first fraction of the bucillamine at a first time point and a second coating that releases a second fraction of the bucillamine at a second time point about two to about six hours after said first time point, said first and second releases occurring in the stomach.

88. The composition according to claim 87, wherein said layers of coatings further comprise a third coating that releases a third fraction of the bucillamine, in the stomach or duodenum, at a third time point about two to about six hours after the second time point.

89. The composition according to any one of claims 77 to 86, wherein the matrix of differently-segments of said modified-release component comprises a first segment that dissolves to release a first fraction of the bucillamine at a first time point and a second segment that dissolves to release a second fraction of the bucillamine at a second time point about two to about six hours after said first time point, said first and second releases occurring in the stomach.

90. The composition according to claim 89, wherein said matrix of differently-dissolving segments further comprises a third segment that dissolves to release a third fraction of the bucillamine, in the stomach or duodenum, at a third time point about two to six eight hours after the second time point.

91. The composition according to any one of claims 77 to 86, wherein the differently-coated beads of said modified-release component comprise a first bead coating that releases a first fraction of the bucillamine at a first time point and a second bead coating that releases a second fraction of the bucillamine at a second time point about two to about six hours after said first time point, said first and second releases occurring in the stomach.

92. The composition according to claim 91, wherein the differently-coated beads further comprise a third bead coating that releases a third fraction of the bucillamine, in the stomach or duodenum, at a third time point about two to about six hours after the second time point.

93. The composition according to any one of claims 77 to 86, wherein the differently-dissolving bands of said modified-release component comprise a first band that releases a first fraction of the bucillamine at a first time point and a second band that releases a second fraction of the bucillamine at a second time point about two to about six hours after said first time point, said first and second releases occurring in the stomach.

94. The composition according to claim 93, wherein the differently-dissolving bands further comprise a third band that releases a third fraction of the bucillamine, in the stomach or duodenum, at a third time point about two to about six hours after the second time point.

95. The composition according to any one of claims 77 to 86, wherein the differently-releasing plugs of said modified-release component comprise a first plug that releases a first fraction of the bucillamine at a first time point and a second plug that releases a second fraction of the bucillamine at a second time point about two to about six hours after said first time point, said first and second releases occurring in the stomach.

96. The composition according to claim 95, wherein the differently-releasing plugs further comprise a third plug that releases a third fraction of the bucillamine, in the stomach or duodenum, at a third time point about two to about six hours after the second time point.

97. The composition according to any one of claims 77 to 86, wherein the differently-expanding osmotic-push compartments of said modified-release component comprise a first osmotic-push compartment that releases a first fraction of the bucillamine at a first time point and a second osmotic-push compartment that releases a second fraction of the bucillamine at a second time point about two to about six hours after said first time point, said first and second releases occurring in the stomach.

98. The composition according to claim 97, wherein the differently-expanding osmotic-push compartments further comprise a third osmotic-push compartment that releases a third fraction of the bucillamine, in the stomach or duodenum, at a third time point about two to about six hours after the second time point.

99. The composition according to any one of claims 77 to 86, wherein the differently-releasing polymers of said modified-release component comprise a first polymer that releases a first fraction of the bucillamine at a first time point and a second polymer that releases a second fraction of the bucillamine at a second time point about two to about six hours after said first time point, said first and second releases occurring in the stomach.

100. The composition according to claim 99, wherein the differently-releasing polymers further comprise a third polymer that releases a third fraction of the bucillamine, in the stomach or duodenum, at a third time point about two to about six hours after the second time point.

101. The composition according to any one of claims 77 to 86, wherein the differently-adhering polymers of said modified-release component comprise a first polymer that releases a first fraction of the bucillamine at a first time point and a second polymer that releases a second fraction of the bucillamine at a second time point about two to about six hours after said first time point, said first and second releases occurring in the stomach.

102. The composition according to claim 101, wherein the differently-adhering polymers further comprise a third polymer that releases a third fraction of the bucillamine, in the stomach or duodenum, at a third time point about two to about six hours after the second time point.

103. The composition according to any one of claims 87 to 102, wherein said second time point is about four hours after said first time point.

104. The composition according to any one of claims 71 to 75, wherein the composition is formulated for transdermal delivery or transmucosal delivery.

105. The composition according to claim 104, wherein the transdermal formulation is provided in a dermal patch.

106. The pharmaceutical composition according to any one of claims 71 to 105 in an effective amount for use in treating or preventing an inflammatory condition or a symptom thereof, in a subject in need thereof.

107. A method of treating or preventing an inflammatory condition or a symptom thereof, in a subject in need thereof, comprising administering to the subject an effective amount of the pharmaceutical composition according to any one of claims 71 to 105.

108. A method of administering a therapeutically effective amount of bucillamine to achieve a therapeutic effect in a subject with an inflammatory condition or a symptom thereof, the method comprising:
administering to said subject a pharmaceutical composition comprising bucillamine, in a therapeutically effective treatment dose, and a pharmaceutically acceptable carrier;
re-administering to said subject said pharmaceutical composition about two to about six hours after said first administration; and
repeating said administering steps to provide an average total daily dose of bucillamine for said condition.

109. A pharmaceutical composition for use in administering a therapeutically effective amount of bucillamine to achieve a therapeutic effect in a subject with an inflammatory condition or a symptom thereof,
said composition comprising a pharmaceutically acceptable carrier and bucillamine in a therapeutically effective treatment dose for repeated administration to said subject, in about two to about six hour intervals, to provide an average total daily dose of bucillamine for said condition.

110. The method according to claim 108, or pharmaceutical composition for use according to claim 109, wherein said condition is rheumatoid arthritis or a related condition and said average total daily dose is 300 mg or less of bucillamine.

111. The method or pharmaceutical composition for use according to claim 110, wherein said average total daily dose is 200 mg or less of bucillamine.

112. The method or pharmaceutical composition for use according to claim 111, wherein said average total daily dose is 100 mg or less of bucillamine.

113. The method according to claim 108, or pharmaceutical composition for use according to claim 109, wherein said condition is gout or a related condition and said average total daily dose is 250 mg or less.

114. The method or pharmaceutical composition for use according to claim 113, wherein said average total daily dose is 100 mg or less.

115. The method according to claim 108, or pharmaceutical composition for use according to claim 109, or the method or pharmaceutical composition for use according to any one of claims 110 to 114, wherein said composition is an immediate-release composition for oral administration.

116. The method according to claim 108, or pharmaceutical composition for use according to claim 109, or the method or pharmaceutical composition for use according to any of claims 110 to 115, wherein said composition further comprises a gastro-retentive feature.

117. The method according to claim 108, or pharmaceutical composition for use according to claim 109, or the method or pharmaceutical composition for use according to any one of claims 110 to 116, wherein said interval is no less than about three hours and said composition is administered no more than about eight times a day.

118. The method or pharmaceutical for use according to claim 117, wherein said interval is four hours and said composition is administered six times a day.

119. A method of administering a therapeutically effective amount of bucillamine to achieve an extended duration therapeutic effect in a subject with an inflammatory condition or a symptom thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about two to about six hours;
wherein dosing is repeated to provide an average total daily dose of bucillamine that is therapeutically effective for said condition; and
wherein said first treatment dose achieves the extended duration therapeutic effect in said subject for at least about an 8-hour duration of effect, optionally, before a second or subsequent treatment dose is administered.

120. A method of treating or preventing an inflammatory condition or a symptom thereof, with extended duration of effect, in a subject in need thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about two to about six hours;
wherein dosing is repeated to provide an average total daily dose of bucillamine that is therapeutically effective for said condition; and
wherein said first treatment dose provides at least about an 8-hour duration of effect in said subject,
thereby extending treatment interval duration for said subject.

121. A pharmaceutical composition in a formulation for use in administering a therapeutically effective amount of bucillamine to achieve an extended duration therapeutic effect in a subject with an inflammatory condition or a symptom thereof,
said composition comprising bucillamine, in a first treatment dose, and a pharmaceutically acceptable modified-release component that releases fractions of the bucillamine in repeated pulses over time at intervals of about two to about six hours;
wherein dosing is repeated to provide an average total daily dose of bucillamine that is therapeutically effective for said condition; and
wherein said first treatment dose achieves the extended duration therapeutic effect in said subject for at least about an 8-hour duration of effect.

122. A pharmaceutical composition in a formulation for use in treating or preventing an inflammatory condition or a symptom thereof, with extended duration of effect, in a subject in need thereof, said composition comprising:
bucillamine, in a first treatment dose, and a pharmaceutically acceptable modified-release component that releases fractions of the bucillamine in repeated pulses over time at intervals of about two to about six hours;
wherein dosing is repeated to provide an average total daily dose of bucillamine that is therapeutically effective for said condition; and
wherein said first treatment dose provides at least about an 8-hour duration of effect in said subject,
thereby extending treatment interval duration for said subject.

123. The method according to claim 119 or claim 120, or the pharmaceutical composition for use according to claim 121 or claim 122, wherein said condition is rheumatoid arthritis or a related condition and said average total daily dose is 300 mg or less.

124. The method or pharmaceutical composition for use according to claim 123, wherein said average total daily dose is 200 mg or less.

125. The method or pharmaceutical composition for use according to claim 124, wherein said average total daily dose is 100 mg or less.

126. The method according to claim 119 or claim 120, or the pharmaceutical composition for use according to claim 121 or claim 122, wherein said condition is gout or a related condition, said extended duration therapeutic effect comprises reducing serum urate concentration to effectively below about 6 mg/dL in said subject, and said average total daily dose is 250 mg or less.

127. The method or pharmaceutical composition for use according to claim 126, wherein said average total daily dose is 100 mg or less.

128. The method according to claim 119 or claim 120, or the pharmaceutical composition for use according to claim 121 or claim 122, or the method or pharmaceutical composition for use according to any one of claims 123 to 127, wherein release of at least said first fraction occurs in the stomach.

129. The method or pharmaceutical composition for use according to claim 128, wherein release of at least said first and second fractions occur in the stomach.

130. The method according to claim 119 or claim 120, the pharmaceutical composition for use according to claim 121 or claim 122, or the method or pharmaceutical composition for use according to any one of claims 123 to 129, wherein the composition achieves the extended duration effect for at least about 12 hours.

131. The method or the pharmaceutical composition for use according to claim 130, wherein the composition achieves the extended duration effect for at least about 24 hours.

132. The method according to claim 119 or claim 120, or the pharmaceutical composition for use according to claim 121 or claim 122, or the method or pharmaceutical composition for use according to any one of claims 123 to 131, wherein the treatment dose is administered no more than twice a day.

133. The method according to claim 119 or claim 120, or pharmaceutical composition for use according to claim 121 or claim 122, or the method or pharmaceutical composition for use of any one of claims 123 to 132, wherein the composition achieves a peak blood level of total bucillamine of no more than about 30,000 ng/mL.

134. The method or pharmaceutical composition for use according to claim 133, wherein the composition achieves a peak blood level of total bucillamine of no more than about 25,000 ng/mL.

135. The method according to claim 119 or claim 120, or pharmaceutical composition for use according to claim 121 or claim 122, or the method or pharmaceutical composition for use of any one of claims 123 to 134, wherein the composition achieves plasma free bucillamine concentration in a range from about 500 to about 3,500 ng/mL for the at least about 8 hours of extended duration of effect.

136. The method or pharmaceutical composition for use according to claim 135, wherein the composition achieves plasma free bucillamine concentration in a range from about 700 to about 2,500 ng/mL for at least about 12 hours of extended duration of effect.

137. The method according to claim 119 or claim 120, or the pharmaceutical composition for use according to claim 121 or claim 122, or the method or pharmaceutical composition for use according to any one of claims 123 to 136, wherein said composition is formulated for oral delivery.

138. The method or pharmaceutical composition for use according to claim 137, wherein the modified-release component of the oral formulation is at least one selected from the group consisting of: layers of coatings; a matrix comprising differently-dissolving segments, a capsule comprising differently-coated beads, a capsule comprising differently-dissolving bands, a capsule comprising differently-releasing plugs, a capsule comprising differently-expanding osmotic-push compartments, a matrix comprising differently-releasing polymers, matrix comprising differently-adhering polymers, and a gastro-retentive feature.

139. The method or pharmaceutical composition for use according to claim 138, wherein said modified-release component comprises said gastro-retentive feature.

140. The method or pharmaceutical composition for use according to claim 139, wherein said gastro-retentive feature is at least one selected from the group consisting of a stomach-adhering coating, a floatation system, a sedimentation system, and an expandable system.

141. The method according to claim 119 or claim 120, or the pharmaceutical composition for use according to claim 121 or claim 122, or the method or pharmaceutical composition for use of any one of claims 123 to 136, wherein said composition is formulated for transmucosal or transdermal delivery.

142. A method of making the pharmaceutical composition according to any one of claims 71 to 105, said method comprising:
providing a therapeutically effective amount of bucillamine for a given condition; and
providing a pharmaceutically acceptable modified-release component that releases fractions of the bucillamine in repeated pulses over time at intervals of about two to about six hours, and
combining the bucillamine and the modified-release component to provide an oral, transdermal, or transmucosal formulation.

143. The method according to claim 142, wherein said modified-release component comprises a gastro-retentive feature for combination with the bucillamine to provide said oral formulation.

144. A method of administering a therapeutically effective amount of bucillamine to achieve a reduced peak blood level in a subject with an inflammatory condition or a symptom thereof, the method comprising:
administering to said subject a pharmaceutical composition comprising bucillamine, in a first treatment dose, and a pharmaceutically acceptable modified-release component that releases fractions of the bucillamine in repeated pulses over time at intervals of about two to about six hours;
wherein said composition achieves a therapeutically effective blood level of free bucillamine, but a reduced peak blood level of total bucillamine of no more than about 30,000 ng/mL; and
wherein dosing is repeated to provide an average total daily dose of bucillamine that is therapeutically effective for said condition.

145. A pharmaceutical composition in a formulation for use in administering a therapeutically effective amount of bucillamine to achieve a reduced peak blood level in a subject with an inflammatory condition or a symptom thereof,
said composition comprising bucillamine, in a first treatment dose, and a pharmaceutically acceptable modified-release component that releases fractions of the bucillamine in repeated pulses over time at intervals of about two to about six hours;
wherein said composition achieves a therapeutically effective blood level of free bucillamine, but a reduced peak blood level of total bucillamine of no more than about 30,000 ng/mL; and
wherein dosing is repeated to provide an average total daily dose of bucillamine that is therapeutically effective for said condition.

146. The method according to claim 144, or the pharmaceutical composition for use according to claim 145, wherein said condition is rheumatoid arthritis or a related condition, and said average total daily dose is 300 mg or less.

147. The method according or pharmaceutical composition for use according to claim 146, wherein said average total daily dose is 200 mg or less.

148. The method according or pharmaceutical composition for use according to claim 147, wherein said average total daily dose is 100 mg or less.

149. The method according to claim 144, or the pharmaceutical composition for use according to claim 145, wherein said condition is gout or a related condition, and said average total daily dose is 250 mg or less.

150. The method or pharmaceutical composition for use according to claim 149, wherein said average total daily dose is 100 mg or less.

151. The method according to claim 144, or the pharmaceutical composition for use according to claim 145, or the method or pharmaceutical composition for use according to any of claims 146 to 150, wherein said reduced peak blood level results in fewer side effects than administration of the same dose of bucillamine in an immediate-release formulation given in one administration or divided amongst two or three administrations per day.
